(19) United States
(12) Patent Application Publication
(10) Pub. No.: US 2022/0347187 A1
(43) Pub. Date: Nov. 3, 2022

(54) HETEROCYCLIC COMPOUNDS AS KINASE INHIBITORS

(71) Applicant: Nuvation Bio Inc., New York, NY (US)

(72) Inventors: Sarvajit CHAKRAVARTY, Edmond, OK (US); Son Minh PHAM, San Francisco, CA (US); Jayakanth KANKANALA, Plymouth, MN (US); Jiyun CHEN, Moraga, CA (US); Brahmam PUJALA, Greater Noida (IN); Bhawana BHATT, Noida (IN); Mukesh GANGAR, Noida (IN); Amit S. SHETE, Noida (IN)

(21) Appl. No.: 17/634,698

(22) PCT Filed: Aug. 13, 2020
(86) PCT No.: PCT/US20/46233

§ 371 Date: Feb. 11, 2022

Related U.S. Application Data
(60) Provisional application No. 62/886,792, filed on Aug. 14, 2019.

(51) Int. Cl. A61K 31/551; C07D 413/14; A61K 31/538; A61P 35/00

(52) U.S. Cl. CPC A61K 31/551; C07D 413/14; A61K 31/538; A61P 35/00

(57) ABSTRACT

Heterocyclic compounds as CDK4 or CDK6 or other CDK inhibitors are provided. The compounds may find use as therapeutic agents for the treatment of diseases and may find particular use in oncology.

### CROSS REFERENCE TO RELATED APPLICATION

This application claims priority to U.S. Provisional Application No. 62/886,792, filed on Aug. 14, 2019, the content of which is incorporated herein by reference in its entirety.

### FIELD OF THE INVENTION

This disclosure relates generally to therapeutics which play a crucial role in the control of the cell cycle and more particularly, compounds that inhibit cyclin-dependent kinases (CDK). The invention also provides pharmaceutically acceptable compositions comprising compounds of the present invention and methods of using said compositions in the treatment of diseases associated with these pathways.

### BACKGROUND OF THE INVENTION

The cell cycle is a period between the successive divisions of a cell. During this period, the contents of the cell must be accurately replicated. The processes that permit the cell to divide are very precisely controlled by a multitude of enzymatic reactions amongst which the protein kinase-triggered protein phosphorylation plays a major role. In eukaryotes, there are four main stages/phases of cell cycle namely the Gap-1 (G1) phase, Synthesis (S) phase, Gap-2 (G2) and Mitosis (M) phases. An extended phase of Gap-1 phase is coined as Gap-0 (G0) phase or Resting phase (Cancers 2014, 6, 2224-2242).

Uncontrolled proliferation is the hallmark of cancer and other proliferative disorders and abnormal cell cycle regulation is, therefore, common in these diseases. Cyclin-dependent kinases (CDK) constitute a heterodimeric family of serine/threonine protein kinases involved in cell cycle and transcription. They include two main groups: cell cycle CDK and transcriptional CDK. The functionality of CDK depends on specific interactions with regulatory proteins named cyclins which form heterodimeric complexes with their partners. These complexes are important regulators of the cellular processes, especially in the cell cycle progression.

The human proteome contains 20 CDK along with 29 cyclins. CDK1. CDK2, CDK4 and CDK6 are generally considered cell cycle CDK, whereas CDK7, CDK8, CDK9 and CDK11 are mainly involved in transcription regulation (Genome Biol 2014; 15(6):122, Nat Cell Biol 2009; 11(11):1275-6). CDK5 is the prototype of atypical CDK: it is activated by the non-cyclin proteins p35 (or Cdk5R1) and p39 (or Cdk5R2) and has unique post-mitotic functions in neuronal biology, angiogenesis and cell differentiation. Proliferative signals induce the transition from the G0 or G1 phases into S phase through the activation of the structurally related CDK4 and CDK6 [Development, 2013; 140 (15):3079-93, Biochem Pharmacol 2012; 84(8):985-93, Nature 2014; 510(7505):393-61. The binding of cyclin D to CDK4 and to CDK6 promotes the phosphorylation of the transcriptional repressor retinoblastoma protein (RB1).

CDK hyperactivity is often observed in cancer, reflecting their prominent role in cell cycle and transcription regulation. In cancer cells, the process of cell division becomes unregulated, resulting in uncontrolled growth that leads to the development of a tumor. A number of mechanisms contribute to the dysregulation of the cell cycle in malignant cells, including the amplification and hyperactivity of CDK4/6, or their genomic instability, which might cause CDK4/6 to become oncogenic drivers of cell replication. Usurping these mechanisms, cancer cells can continue to replicate by triggering the G1 to S phase transition. This process appears to be facilitated by a shortening of the G1 phase. In a cancer cell, CDK4/6 antagonizes intrinsic tumor suppression mechanisms including cell senescence and apoptosis, which further augments the growth of a tumor. Cancer cells also upregulate other CDK and cyclins and decrease suppressive mechanisms such as intrinsic CDK inhibitors and tumor suppressor proteins. The overall effect of this type of cell cycle dysregulation is malignant cell proliferation and the development of cancer (Clinical Breast Cancer, 2016, 1526-8209).

Several CDK inhibitors have been reported (such as in WO2011101409 and WO2011101417) or clinically developed. Flavopiridol and R-Roscovitine (Seliciclib), were the first generation of pan-CDK inhibitors with anti-tumor activity attributed to down-regulation of CDK9-mediated anti-apoptotic proteins, especially Mcl-1. Recently, a new generation of CDK inhibitors have been developed, advanced to clinical trials, and approved for certain types of cancer. Dinaciclib, a selective inhibitor of CDK1, CDK2, CDK5, and CDK9, was directed towards refractory chronic lymphocytic leukemia while palbociclib was tested against advanced estrogen receptor (ER)-positive breast cancer as a selective inhibitor of CDK4 and CDK6. The development of more selective second and third generation CDK inhibitors, including specific CDK4/6 inhibitors, has led to a renewed enthusiasm for manipulating the cyclin D1-CDK4/6 axis in cancer treatment. There are three FDA-approved CDK4/6 inhibitors presently: Palbociclib, Ribociclib and Abemaciclib.

The development of therapies, including monotherapies, for treatment of proliferative disorders using a therapeutic targeted generically at CDK, or specifically at dual inhibition of CDK4 and CDK6, is therefore potentially highly desirable.

There is still a need for new CDK4/6 inhibitors. Compounds for the treatment of hyper-proliferative diseases preferably have at least one advantageous property selected from selectivity, potency, stability, pharmacodynamic properties and safety profile. In this regard, a novel class of CDK4/6 inhibitors is provided herein.

### BRIEF SUMMARY OF THE INVENTION

In one embodiment, provided is a compound of Formula (I):

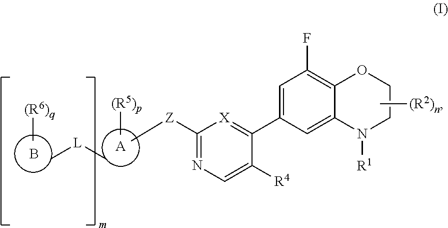

or a salt thereof, wherein X, Z, A, B, L, R¹, R², R⁴, R⁵, R⁶, m, n, p and q are as detailed herein.

In another aspect, provided is a method of treating cancer in an individual in need thereof comprising administering to the individual a therapeutically effective amount of a compound as detailed herein, such as a compound of any one of Formula (I), (I-A), (I-B1) to (I-B20), (I-C1) to (I-C45), or a pharmaceutically acceptable salt thereof. Also provided is a method of modulating CDK4/6 in an individual, comprising administering to the individual a compound detailed herein, or a salt thereof. Also provided is a method of modulating CDK4/6 and one or more of CDK1, CDK2, and CDK9 in an individual, comprising administering to the individual a compound detailed herein, or a salt thereof. Also provided is a method of inhibiting CDK4/6 in a cell, comprising administering a compound detailed herein, or a salt thereof, to the cell. Also provided is a method of inhibiting CDK4/6 and one or more of CDK1, CDK2, and CDK9 in a cell, comprising administering a compound detailed herein, or a salt thereof, to the cell. In some embodiments of the methods detailed herein, the methods comprise administration of a compound detailed herein, or a salt thereof, as a monotherapy.

In another aspect, provided is a pharmaceutical composition comprising a compound detailed herein, or a pharmaceutically acceptable salt thereof, and a pharmaceutically acceptable carrier or excipient. Kits comprising a compound detailed herein, or a salt thereof, are also provided. Kits may optionally include instructions for use, such as instructions for use in any of the methods detailed herein, for example, for use in the treatment of cancer. A compound as detailed herein, or a salt thereof, is also provided for the manufacture of a medicament for the treatment of cancer.

### DETAILED DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C═C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantyl, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted by more than one halogen, it may be referred to by using a prefix corresponding to the number of halogen moieties attached, e.g., dihaloaryl, dihaloalkyl, trihaloaryl etc. refer to aryl and alkyl substituted by two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, pyrazolyl, oxazolyl, isooxazolyl, imidazolyl, quinolyl, isoquinolyl, benzimidazolyl, benzpyrazolyl, benzotriazolyl, indole, benzothiazyl, benzoxazolyl, benzisoxazolyl, imidazopyridinyl and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, dihydrooxazolyl, dihydroisoxazolyl, dioxolanyl, morpholinyl, dioxanyl, tetrahydrothiophenyl, and the like.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different, provided that the group's normal valence is not exceeded. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

As used herein “CDK” refers to one or more cyclin-dependent kinases. CDK4/6 refers to both CDK4 and CDK6. Thus, inhibitors of CDK4/6 inhibit both CDK4 and CDK6.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that embodiments, aspects and variations described herein also include “consisting” and/or “consisting essentially of” embodiments, aspects and variations.

### Compounds

In one aspect, provided is a compound of the Formula (I):

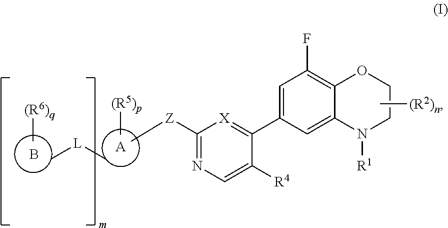

or a salt thereof, wherein:

Z is —NH—, —C(O)NH—, —NH(CO)—, —S(O)₂NH—, or —NHS(O)₂—;

X is N or CR^{a}, wherein R^{a} is hydrogen or —CN;

A is C₃-C₆ cycloalkyl, 4- to 7-membered heterocyclyl, 5- to 7-membered heteroaryl, or C₆ aryl, each of which is optionally substituted by R⁵;

L is a bond. —(CR¹¹R¹²)ᵣ—, —CR¹¹R¹²—O—, —O—, —S—, —S(O)₂—, —C(O)—, —NR¹⁰—, —S(O)₂NR¹⁰—or NR¹⁰S(O)₂—, wherein r is 1, 2 or 3;

B is hydrogen. C₃-C₁₂ cycloalkyl, or 3- to 12-membered heterocyclyl, wherein the C₃-C₁₂cycloalkyl and 3- to 12-membered heterocyclyl of B are each independently optionally substituted by R⁶;

R¹ is C₁-C₆ alkyl, C₂-C₆ alkenyl, C₂-C₆ alkynyl, C₃-C₆ cycloalkyl, 3- to 12-membered heterocyclyl, —(C₁-C₃ alkylene)(C₃-C₆ cycloalkyl), or —(C₁-C₃ alkylene)(3- to 12-membered heterocyclyl), each of which is independently optionally substituted by halogen, —OR¹³, —NR¹³R¹⁴, —C(O)R¹³, —CN, C₃-C₈ cycloalkyl, or C₁-C₆ alkyl optionally substituted by oxo, —OH or halogen, provided that when n is 1 and R² is oxo, then R¹ is C₂-C₆ alkyl, C₂-C₆ alkenyl, C₂-C₆ alkynyl, C₃-C₆ cycloalkyl, 3- to 12-membered heterocyclyl, or —(C₁-C₃ alkylene)(C₃-C₆ cycloalkyl), each of which is independently optionally substituted by halogen, —OR¹³, —C(O)R¹³, —CN, C₃-C₈ cycloalkyl, or C₁-C₆ alkyl optionally substituted by oxo, —OH or halogen;

each R² is independently C₁-C₆ alkyl, oxo, —NR¹¹R¹², —CN, —C(O)R¹⁰, —C(O)NR¹¹R¹² or halogen, wherein any two R² groups are independently attached to same carbon or two different carbons;

R⁴ is hydrogen, C₁-C₆ alkyl, C₃-C₆ cycloalkyl, C₁-C₆ haloalkyl, C₁-C₆ alkoxy, C₁-C₆ haloalkoxy, halogen, —CN, or —OH;

each R⁵ is independently C₁-C₆ alkyl, C₂-C₆ alkenyl. C₂-C₆ alkynyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹¹C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃alkylene)NR¹¹S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), wherein the C₁-C₆ alkyl, C₂-C₆ alkenyl, C₂-C₆ alkynyl, —OR¹¹, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃alkylene)NR¹⁰S(O)₂R¹¹, —(C₁-C₃ alkylene)NR¹⁰S(O)₂NR¹¹R¹², —(C₁-C₃alkylene)S(O)₂NR¹¹R¹², —(C₁-C₃ alkylene)(C₃-C₆ cycloalkyl), and —(C₁-C₃ alkylene)(3- to 12-membered heterocyclyl) of R are each independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R³, C₃-C₈ cycloalkyl, or C₁-C₆ alkyl optionally substituted by oxo, —OH or halogen;

each R⁶ is independently oxo, halogen, or R⁷.

R⁷ is independently hydrogen, C₁-C₆ alkyl, C₂-C₆ alkenyl, C₂-C₆ alkynyl, C₃-C₆ cycloalkyl, 3- to 6-membered heterocyclyl, —OR¹⁰, —NR¹¹R¹², —NR¹⁰C(O)R¹¹, —NR¹⁰C(O)NR¹¹R¹², —S(O)₂R¹⁰, —NR¹⁰S(O)₂R¹¹, —S(O)₂NR¹¹R¹², —C(O)R¹⁰, —C(O)NR¹¹R¹², —(C₁-C₃ alkylene)CN. —(C₁-C₃ alkylene)OR¹⁰, —(C₁-C₃ alkylene)SR¹⁰, —(C₁-C₃ alkylene)NR¹¹R¹², —(C₁-C₃ alkylene)CF₃, —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)S(O)₂NR¹¹R¹², (C₁-C₃ alkylene)(C₃-C₆ cycloalkyl), or —(C₁-C₃ alkylene)(3- to 6-membered heterocyclyl), wherein the C₁-C₆ alkyl, C₂-C₆ alkenyl, C₂-C₆ alkynyl, C₃-C₆ cycloalkyl, 3- to 6-membered heterocyclyl, —OR¹⁰. —NR¹¹R¹², —NR¹⁰C(O)R¹¹, —NR¹⁰C(O)NR¹¹R¹², —S(O)₂R¹⁰, —NR¹⁰S(O)₂R¹¹, —S(O)₂NR¹¹R¹², —C(O)R¹⁰, —C(O)NR¹¹R¹²—, —(C₁-C₃ alkylene)CN. —(C₁-C₃ alkylene)OR¹⁰, —(C₁-C₃ alkylene)SR¹⁰, —(C₁-C₃ alkylene)NR¹¹R¹², —(C₁-C₃ alkylene)CF₃, —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)S(O)₂NR¹¹R¹², (C₁-C₃ alkylene)(C₃-C₆ cycloalkyl), and —(C₁-C₃ alkylene)(3- to 6-membered heterocyclyl) of R⁷ are each independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₅ cycloalkyl, or C₁-C₆ alkyl optionally substituted by oxo, —OH or halogen;

R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl or 3- to 6-membered heterocyclyl, wherein the C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, and 3- to 6-membered heterocyclyl of R¹⁰ are each independently optionally substituted by halogen, oxo. —CN, —OR¹⁵, —NR¹⁵R¹⁶, or C₁-C₆ alkyl optionally substituted by halogen, —OH or oxo;

R¹¹ and R¹² are each independently hydrogen, C₁-C₆ alkyl. C₃-C₆ cycloalkyl, —(C₁-C₃alkylene)(C₃-C₆ cycloalkyl). C₆-C₁₄ aryl, 5- to 6-membered heteroaryl or 3- to 6-membered heterocyclyl, wherein the C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, and 3- to 6-membered heterocyclyl of R¹¹ and R¹² are each independently optionally substituted by halogen, oxo, —CN, —OR¹⁵, —NR¹⁵R¹⁶ or C₁-C₆ alkyl optionally substituted by halogen, —OH or oxo;

R¹³ and R¹⁴ are each independently hydrogen or C₁-C₆ alkyl, wherein the C₁-C₆ alkyl of R¹³ and R¹⁴ are optionally substituted by halogen, —OR¹⁵, —NR¹⁵R¹⁶, or oxo;
or R¹³ and R¹⁴ are taken together with the atom to which they attached to form a 3- to 6-membered heterocyclyl optionally substituted by halogen, oxo or C₁-C₆ alkyl optionally substituted by halogen or oxo; and

R¹⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R¹⁵ and R¹⁶ are taken together with the atom to which they attached to form a 3- to 6-membered heterocyclyl optionally substituted by halogen, oxo or C₁-C₆ alkyl optionally substituted by oxo or halogen;

p and q are each independently 0, 1, 2 or 3;

m is 0 or 1; and

n is 0, 1, 2, 3 or 4.

In some embodiments of a compound of Formula (I), the compound is other than the compounds in Table 1X, or a tautomer or isomer thereof, or a salt of any of the foregoing.

TABLE 1X

| Comp. No. | Name |
|---|---|
| 1X | 4-[4-(4-aminocyclohexyl)-8-fluoro-3,4-dihydro-2H-1,4-benzoxazin-6-yl]- |
| | N-(5-[(4-ethylpiperazin-1-yl)methyl]pyridin-2-yl}-5-fluoropyrimidin-2- |
| | amine |
| 2X | 4-({5-fluoro-4-[8-fluoro-4-(propan-2-yl)-3,4-dihydro-2H-1,4-benzoxazin- |
| | 6-yl]pyrimidin-2-yl}amino)-3',6'-dihydro-1'H,2H,2'H-[1,4'-bipyridin]-2- |
| | one |
| 3X | 4-({5-fluoro-4[8-fluoro-4-(propan-2-yl)-3,4-dihydro-2H-1,4-benzoxazin- |
| | 6-yl]pyrimidin-2-yl}amino)-1-(piperidin-4-yl)-1,2-dihydropyridin-2-one |
| 4X | 5-fluoro-4-[8-fluoro-4-(propan-2-yl)-3,4-dihydro-2H-1,4-benzoxazin-6- |
| | yl]-N-(3-fluoro-4-(octahydropyrrolo[1,2-a]pyrazin-2- |
| | yl)phenyl)pyrimidin-2-amine |
| 5X | 2-{1-[6-({5-fluoro-4-[8-fluoro-4-(propan-2-yl)-3,4-dihydro-2H-1,4- |
| | benzoxazin-6-yl]pyrimidin-2-yl}amino)pyridin-3-yl]piperidin-4- |
| | yl}ethan-1-ol |
| 6X | 5-fluoro-4-[8-fluoro-4-(propan-2-yl)-3,4-dihydro-2H-1,4-benzoxazin-6- |
| | yl]-N-[5-(piperidin-4-yl)pyrazin-2-yl]pyrimidin-2-amine |
| 7X | 5-fluoro-4-[8-fluoro-4-(propan-2-yl)-3,4-dihydro-2H-1,4-benzoxazin-6- |
| | yl]-N-[6-(1-methylpiperidin-4-yl)pyridazin-3-yl]pyrimidin-2-amine |
| 8X | 8-fluoro-6-(5-fluoro-2-{[3-fluoro-4-(1-methylpiperidin-4- |
| | yl)phenyl]amino}pyrimidin-4-yl)-N,N-dimethyl-4-(propan-2-yl)-3,4- |
| | dihydro-2H-1,4-benzoxazin-2-amine |
| 9X | 8-fluoro-6-(5-fluoro-2-{[3-fluoro-4-(1-methylpiperidin-4- |
| | yl)phenyl]amino}pyrimidin-4-yl)-4-(propan-2-yl)-3,4-dihydro-2H-1,4- |
| | benzoxazine-2-carbonitrile |
| 10X | 1-[8-fluoro-6-(5-fluoro-2-{[3-fluoro-4-(1-methylpiperidin-4- |
| | yl)phenyl]amino}pyrimidin-4-yl)-4-(propan-2-yl)-3,4-dihydro-2H-1,4- |
| | benzoxazin-2-yl]ethan-1-one |
| 11X | 8-fluoro-6-(5-fluoro-2-{[3 -fluoro-4-(1-methylpiperidin-4- |
| | yl)phenyl]amino}pyrimidin-4-yl)-N-methyl-4-(propan-2-yl)-3,4-dihydro- |
| | 2H-1,4-benzoxazine-2-carboxamide |
| 12X | 4-({5-fluoro-4-[8-fluoro-4-(propan-2-yl)-3,4-dihydro-2H-1,4-benzoxazin- |
| | 6-yl]pyrimidin-2-yl}amino)-1-[1-(propan-2-yl)piperidin-4-yl]-1,2- |
| | dihydropyridin-2-one |
| 13X | 5-fluoro-4-[8-fluoro-4-(propan-2-yl)-3,4-dihydro-2H-1,4-benzoxazin-6- |
| | yl]-N-(3-fluoro-4-{1-[(1-methylazetidin-3-yl)methyl]piperidin-4- |
| | yl}phenyl)pyrimidin-2-amine |
| 14X | 2-fluoro-N4-{5-fluoro-4-[8-fluoro-4-(propan-2-yl)-3,4-dihydro-2H-1,4- |
| | benzoxazin-6-yl]lpyridin-2-yl}-N1-(oxotan-3-yl)benzene-1,4-diamine |
| 15X | 2-fluoro-N4-{5-fluoro-4-[8-fluoro-4-(propan-2-yl)-3,4-dihydro-2H-1,4- |
| | benzoxazin-6-yl]pyridin-2-yl}-N1-methyl-N1-(oxolan-3-yl)benzene-1,4- |
| | diamine |
| 16X | N4-{4-[4-(butan-2-yl)-8-fluoro-3,4-dihydro-2H-1,4-benzoxazin-6-yl]-5- |
| | fluoropyridin-2-yl}-2-fluoro-N1-methyl-N1-(oxolan-3-yl)benzene-1,4- |
| | diamine |
| 17X | N4-{4-[4-(butan-2-yl)-8-fluoro-3,4-dihydro-2H-1,4-benzoxazin-6-yl]-5- |
| | fluoropyridin-2-yl)-2-fluoro-N1-(oxolan-3-yl)benzene-1,4-diamine |
| 18X | 2-fluoro-N4-{5-fluoro-4-[8-fluoro-2,2-dimethyl-4-(propan-2-yl)-3,4- |
| | dihydro-2H-1,4-benzoxazin-6-yl]pyridin-2-yl}-N1-(oxolan-3-yl)benzene- |
| | 1,4-diamine |
| 19X | 2-fluoro-N4-{5-fluoro-4-[8-fluoro-2,2-dimethyl-4-(propan-2-yl)-3,4- |
| | dihydro-2H-1,4-benzoxazin-6-yl]pyridin-2-yl}-N1-methyl-N1-(oxolan-3- |
| | yl)benzene-1,4-diamine |
| 20X | N4-{4-[4-(butan-2-yl)-8-fluoro-2,2-dimethyl-3,4-dihydro-2H-1,4- |
| | benzoxazin-6-yl]-5-fluoropyridin-2-yl}-2-fluoro-N1-methyl-N1-(oxolan- |
| | 3-yl)benzene-1,4-diamine |
| 21X | N4-{4-[4-(butan-2-yl)-8-fluoro-2,2-dimethyl-3,4-dihydro-2H-1,4- |
| | benzoxazin-6-yl]-5-fluoropyridin-2-yl}-2-fluoro-N1-(oxolan-3- |
| | yl)benzene-1,4-diamine |
| 22X | 4-({5-fluoro-4-[8-fluoro-4-(propan-2-yl)-3,4-dihydro-2H-1,4-benzoxazin- |
| | 6-yl]pyrimidin-2-yl}amino)-N-(piperidin-4-yl)benzene-1-sulfonamide |
| 23X | 5-fluoro-4-(8-fluoro-4-isopropyl-3,4-dihydro-2H-benzo[b][1,4]oxazin-6 - |
| | yl)-N-(5-(1-methylpiperidin-4-yl)-6-(trifluoromethyl)pyridin-2- |
| | yl)pyrimidin-2-amine. |
| 24X | 6-((5-fluoro-4-(8-fluoro-4-isopropyl-3,4-dihydro-2H- |
| | benzo[b][1,4]oxazin-6-yl)pyrimidin-2-yl)amino)-3-(1-methylpiperidin-4- |
| | yl)pceolinonitrile |
| 25X | 5-fluoro-N-(5-(1-methylpiperidin-4-yl)-6-(trifluoromethy)pyridin-2-yl)- |
| | 4-(2,2,8-trifluoro-4-isopropyl-3,4-dihydro-2H-benzo[b][1,4]oxazin-6- |
| | yl)pyrimidin-2-amine |
| 26X | 6-((5-fluoro-4-(2,2,8-trifluoro-4-isopropyl-3,4-dihydro-2H- |
| | benzo[b][1,4]oxazin-6-yOpyrim idi n-2-yl)amino)-3-(1-methylpiperidin-4- |
| | yl)picolinonitrile |
| 27X | 4-(2,8-difluoro-4-isopropyl-3,4-dihydro-2H-benzo[b][1,4]oxazin-6-yl)-5- |
| | fluoro-N-(5-(1-methylpiperidin-4-yl)-6-(trifluoromethyl)pyridin-2- |
| | yl)pyrimidin-2-amine |
| 28X | 6-((4-(2,8-difluoro-4-isopropyl-3,4-dihydro-2H-benzo[b][1,4]oxazin-6- |
| | yl)-5-fluoropyrimidin-2-yl)amino)-3-(1-methylpiperidin-4- |
| | yl)picolinonitrile |
| 29X | 5-fluoro-4-(8-fluoro-4-isopropyl-3,4-dihydro-2H-benzo[b][1,4]oxazin-6- |
| | yl)-N-(5-(1-methylpiperidin-4-yl)-6-(trifluoromethyl)pyridin-2- |
| | yl)pyrimidin-2-amine |
| 30X | 6-((5-fluoro-4(8-fluoro-4-isopropyl-3,4-dihydro-2H- |
| | benzo[b][1,4]oxazin-6-yl)pyrimidin-2-yl)amino)-3-(1-methylpiperidin-4- |
| | yl)picolinonitrile |
| 31X | N-(4-(1,4-diazepan-1-yl)-3-fluorophenyl)-5-fluoro-4-(8-fluoro-4- |
| | isopropyl-3,4-dihydro-2H-benzo[b][1,4]oxazin-6-yl)pyrimidin-2-amine |
| 32X | N-(5-(1,4-diazepan-1-yl)pyridin-2-yl)-5-fluoro-4-(8-fluoro-4-isopropyl- |
| | 3,4-dihydro-2H-benzo[b][1,4]oxazin-6-yl)pyrimidin-2-amine |
| 33X | N-(5-(1,4-diazepan-1-yl)pyridin-2-yl)-4-(4-cyclopentyl-8-fluoro-3,4- |
| | dihydro-2H-benzo[b][1,4]oxazin-6-yl)-5-fluoropyrimidin-2-amine |

Specific values listed below are values for a compound of Formula (I) as well as all related formulae (e.g., Formula (I-A), (I-B1) to (I-B320), and (I-C1) to (I-C45)), or a salt thereof. It is to be understood that two or more values may combined. Thus, it is to be understood that any variable for a compound of Formula (I) as well as all related formulae may be combined with any other variable for a compound of Formula (I) as well as all related formulae the same as if each and every combination of variables were specifically and individually listed. For example, it is understood that any specific value of R¹ detailed herein for a compound of Formula (I) as well as all related formulae may be combined with any other specific value for one or more of the variables A, L, B, X, Z, R¹, R², R⁴, R⁵, R⁶, m, n, p, and q the same as if each and every combination were specifically and individually listed.

In some embodiments of a compound of Formula (I) or a variation thereof, the compound has one or more of the following features:

(1) when the compound is of Formula (X-1)

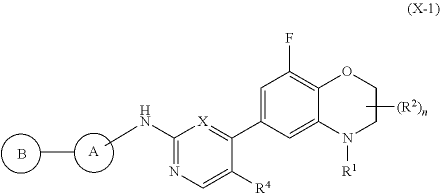

and
when
n is 0, 1, 2, 3 or 4;
R¹ is unsubstituted C₁-C₄ alkyl, unsubstituted C₃-C₆ cycloalkyl, or cyclohexylamine;
R² is methyl, oxo, or fluoro;
R⁴ is methyl, fluoro, chloro, —OCH₃, —CF₃, —OCF₃, or cyclopropyl;
X is CH or N; and
A is phenyl, fluorophenyl, cyanophenyl, pyrazolyl, imidazolyl, oxazolyl, thiazolyl, pyrrolidinyl, piperidinyl, cyclohexylyl, pyridyl, fluoropyridyl, or pyrimidinyl, then
B is other than a moiety selected from the group consisting of

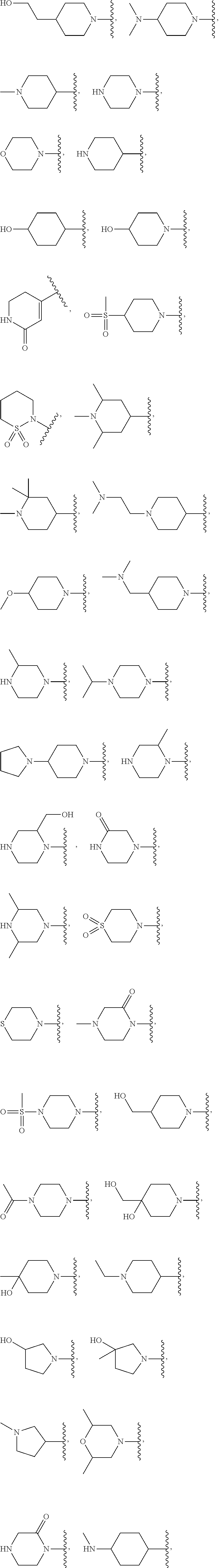

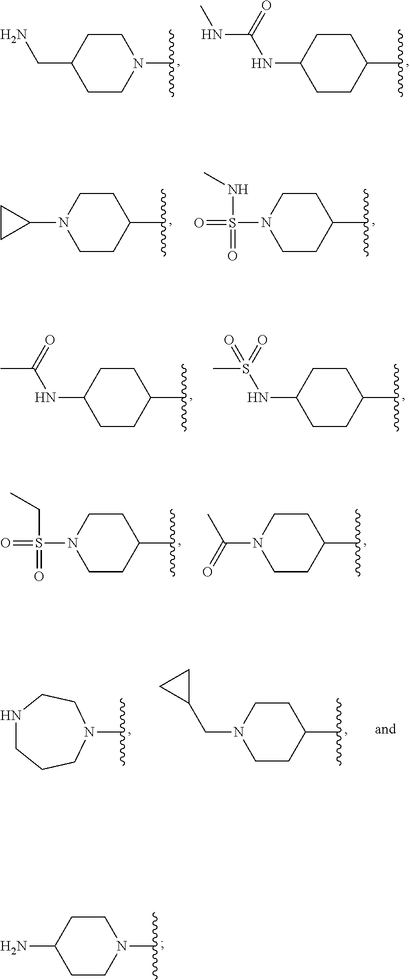

and/or
(2) when the compound is of Formula (X-1)

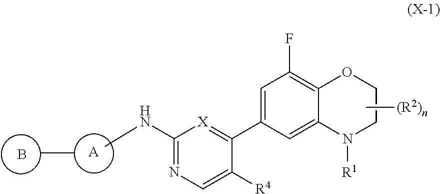

and when B is

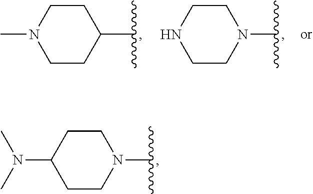

then A is other than a moiety selected from the group consisting of substituted phenyl, unsubstituted pyridyl, and pyridyl which is substituted with fluoro, methoxy, methyl, n-propyl, isopropyl, cyclopropyl, cyclopentyl, or isopropoxy; and/or

(3) when the compound is of Formula (X-2)

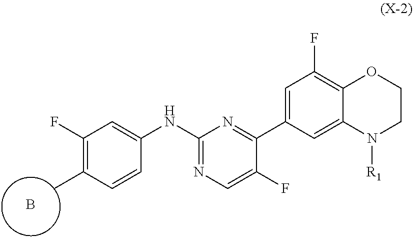

and
when B is

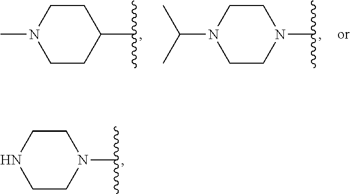

then
R₁ is other than a moiety selected from the group consisting of

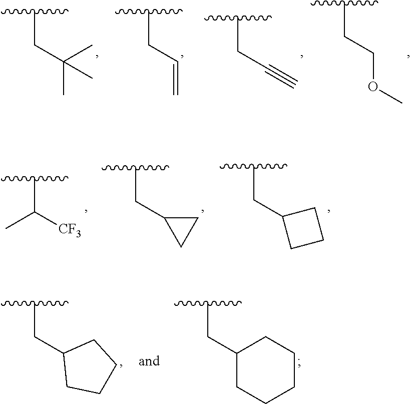

and/or

(4) when the compound is of Formula (X-3):

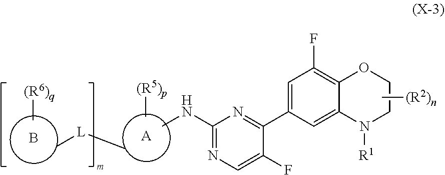

and
when
R¹ is unsubstituted C₃-C₄ alkyl;
R² is methyl;
n is 0 or 2;
m is 0 or 1;
L is —CH₂—, —O—, —S—, —NH—, —N(CH₃)—, —C(O)—, or —S(O)₂—; and
B is

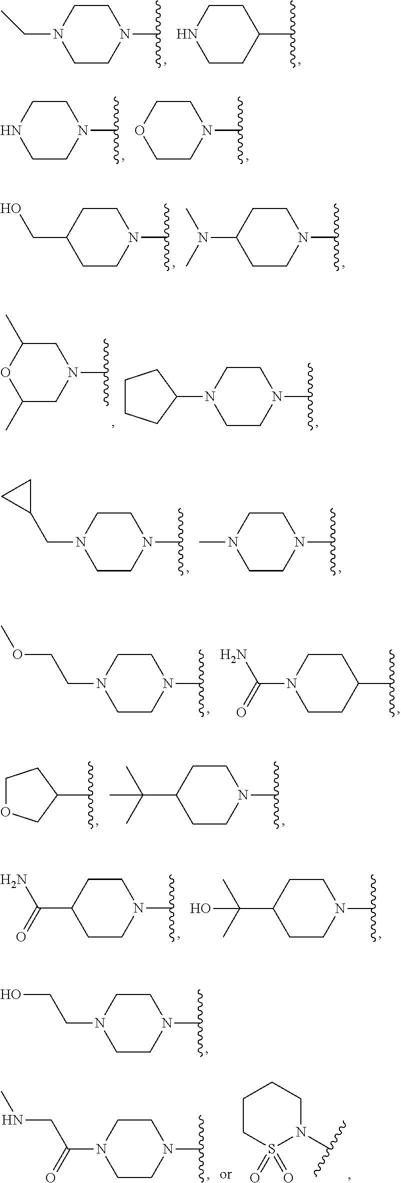

then
A is other than a moiety selected from the group consisting of:

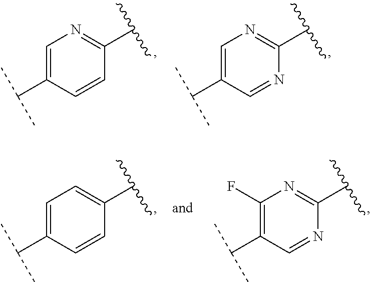

wherein [symbol] and [symbol] are the points of attachment with —NH— and L respectively; and/or

(5) when the compound is of Formula (X-4)

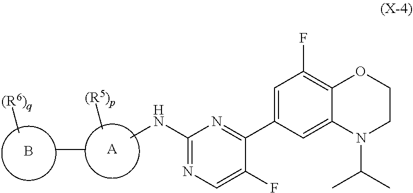

and
when
A is

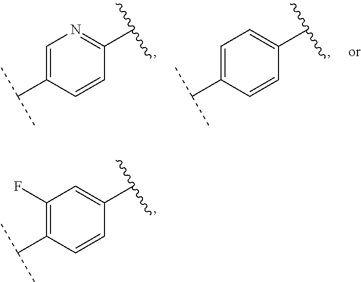

wherein [symbol] and [symbol] are the points of attachment with —NH— and B respectively; and
B is

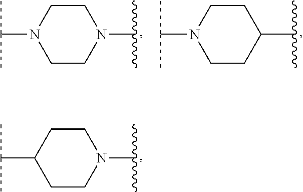

wherein [symbol] and [symbol] are the points of attachment with A and R⁶ respectively, then
R⁶ is other than a moiety selected from the group consisting of:

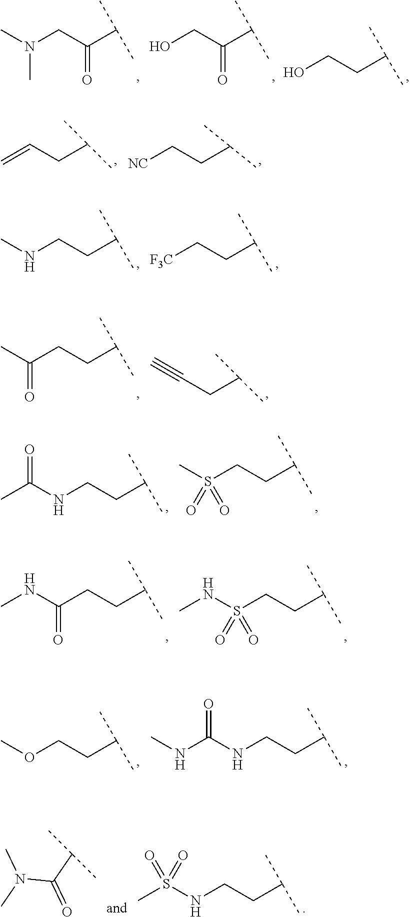

In some embodiments of a compound of Formula (I) or a variation thereof, (1), (2), (3), (4), and (5) apply. In some embodiments, (1) applies. In some embodiments, (2) applies. In some embodiments, (3) applies. In some embodiments, (4) applies. In some embodiments, (5) applies.

In some embodiments of a compound of Formula (I) or a variation thereof, Z is —NH—. In some embodiments, Z is —C(O)NH—. In some embodiments, Z is —NH(CO)—. In some embodiments, Z is —NHS(O)₂—. In some embodiments. Z is —S(O)₂NH—. In some embodiments, Z is —NH—, —NH(CO)—, or —C(O)NH—. In some embodiments. Z is —NH—.

In some embodiments of a compound of Formula (I) or a variation thereof. A is C₃-C₆ cycloalkyl, 4- to 7-membered heterocyclyl, 5- to 7-membered heteroaryl or C₆ aryl, each of which is unsubstituted. In some embodiments, A is C₃-C₆ cycloalkyl, 4- to 7-membered heterocyclyl, 5- to 7-membered heteroaryl, or C₆ aryl, each of which is optionally substituted by R⁵. In some embodiments, A is phenyl optionally substituted by R⁵. In some embodiments, A is 5- to 7-membered heteroaryl optionally substituted by R⁵. In some embodiments, A is 5-membered heteroaryl optionally substituted by R⁵. In some embodiments, A is 6-membered heteroaryl optionally substituted by R⁵. In some embodiments, A is 7-membered heteroaryl optionally substituted by R⁵. In some embodiments, A is pyridyl, pyrimidinyl, pyrazinyl, pyrazolyl, thiazolyl, oxazolyl, isooxazolyl, or imidazolyl, each of which is optionally substituted by R⁵. In some embodiments, A is 4- to 7-membered heterocyclyl optionally substituted by R⁵. In some embodiments, A is 5- to 7-membered heterocyclyl optionally substituted by R⁵. In some embodiments, A is 5- to 6-membered heterocyclyl optionally substituted by R⁵. In some embodiments. A is 4-membered heterocyclyl optionally substituted by R⁵. In some embodiments, A is 5-membered heterocyclyl optionally substituted by R⁵. In some embodiments, A is 6-membered heterocyclyl optionally substituted by R⁵. In some embodiments, A is 7-membered heterocyclyl optionally substituted by R⁵. In some embodiments, A is piperidinyl, pyrrolidinyl, azetidinyl, dihydropyridine, or pyridonyl, each of which is optionally substituted by R⁵. In some embodiments, A is C₃-C₆ cycloalkyl optionally substituted by R⁵. In some embodiments, A is C₃-C₆ cycloalkyl substituted by R⁵. In some embodiments A is cyclohexyl or cyclopentyl, each of which is optionally substituted by R⁵. In some embodiments, A is phenyl, pyridyl, pyrimidinyl, pyrazinyl, pyrazolyl, thiazolyl, oxazolyl, isooxazolyl, imidazolyl, piperidinyl, pyrrolidinyl, azetidinyl, pyridonyl, cyclohexyl, or cyclopentyl, each of which is unsubstituted. In some embodiments of a compound of Formula (I). A is phenyl, pyridyl, pyrimidinyl, pyrazolyl, thiazolyl, oxazolyl, isooxazolyl, imidazolyl, piperidinyl, pyrrolidinyl, azetidinyl, dihydropyridinyl, pyridonyl, cyclohexyl, or cyclopentyl, each of which is optionally substituted by R⁵. In some embodiments. A is phenyl, pyridyl, pyrazinyl, piperidinyl, pyrazolyl, or cyclohexyl, each of which is optionally substituted by R⁵. In some embodiments, A is phenyl optionally substituted by R⁵. In some embodiments. A is pyridyl optionally substituted by R⁵. In some embodiments, A is piperidinyl optionally substituted by R⁵. In some embodiments, A is pyrazolyl optionally substituted by R⁵. In some embodiments, A is cyclohexyl optionally substituted by R⁵. In some embodiments, A is pyrazinyl optionally substituted by R⁵.

In some embodiments of a compound of Formula (I) or a variation thereof, m is 0. In some embodiments, m is 1.

In some embodiments of a compound of Formula (I) or a variation thereof, B is hydrogen. C₃-C₁₂ cycloalkyl, or 3- to 12-membered heterocyclyl, wherein the C₃-C₁₂cycloalkyl and 3- to 12-membered heterocyclyl of B are each independently optionally substituted by R⁶. In some embodiments, B is C₃-C₁₂ cycloalkyl or 3- to 12-membered heterocyclyl, each of which is unsubstituted. In some embodiments, B is hydrogen. In some embodiments, B is 5- to 12-membered heterocyclyl optionally substituted by R⁶. In some embodiments, B is 5- to 12-membered heterocyclyl optionally substituted by R⁶, wherein the 5- to 12-membered heterocyclyl is a spiro, fused, or bridged heterocyclyl. In some embodiments, B is 5- to 12-membered heterocyclyl optionally substituted by R⁶, wherein the 5- to 12-membered heterocyclyl is a spiro heterocyclyl. In some embodiments, B is 5- to 12-membered heterocyclyl optionally substituted by R⁶, wherein the 5- to 12-membered heterocyclyl is a fused heterocyclyl. In some embodiments, B is 5- to 12-membered heterocyclyl optionally substituted by R⁶, wherein the 5- to 12-membered heterocyclyl is a bridged heterocyclyl. In some embodiments of a compound of Formula (I), B is

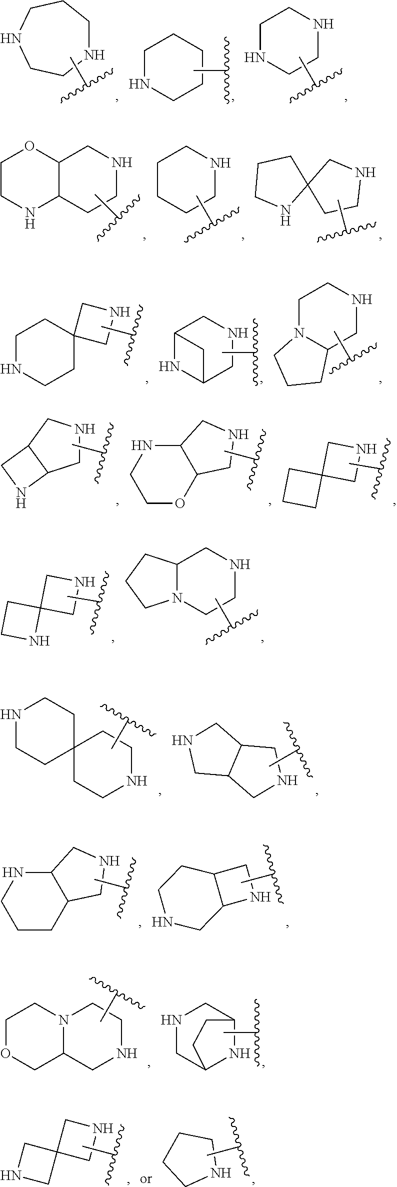

each of which is optionally substituted by R⁶. In some embodiments. B is C₃-C₆ cycloalkyl optionally substituted by R⁶. In some embodiments, B is C₃-C₁₂ cycloalkyl optionally substituted by R⁶. In some embodiments, B is cyclopentyl, cyclohexyl, or cycloheptyl, each of which is optionally substituted by R⁶.

In some embodiments of a compound of Formula (I), L is a bond, —(CR¹¹R¹²)ᵣ—, —CR¹¹R¹²—O—, —O—, —S—, —S(O)₂—, —C(O)—, —NR¹⁰—, —S(O)₂NR¹⁰—, or —NR¹⁰S(O)₂—. In some embodiments of a compound of Formula (I). L is a bond, —CH₂—, —NH—, —O—, —S—, —S(O)₂—, —C(O)—, —NCH₃—, —S(O)₂NH—, or —NHS(O)₂—. In some embodiments of a compound of Formula (I), L is a bond, —CH₂—, —NH—, —O—, or —S—. In some embodiments, L is a bond. In some embodiments, L is —CH₂—. In some embodiments, L is —NH—. In some embodiments. L is —S—. In some embodiments, L is —O—. In some embodiments, L is —S(O)₂—. In some embodiments, L is —C(O)—. In some embodiments, L is —NCH₃—. In some embodiments, L is —NHS(O)₂—. In some embodiments, L is —CR¹¹R¹²—. In some embodiments, L is —NR¹⁰—. In some embodiments, L is —NR¹⁰S(O)₂—. In some embodiments. L is —NHS(O)₂—. In some embodiments, L is —S(O)₂NR¹⁰—. In some embodiments, L is —S(O)₂NH—.

It is understood that any description of A for Formula (I) may be combined with any description of B and L for formula (I), the same as if each and every combination were specifically and individually listed.

In some embodiments, provided is a compound of Formula (I-A),

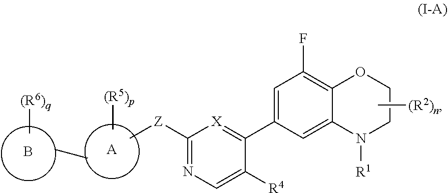

or a salt thereof, wherein A, B, X, Z, R¹, R², R⁴, R⁵, R⁶, n, p, and q are as detailed herein for Formula (I).

In some embodiments, provided is a compound of any one of Formula (I-B1) to (I-B20), or a salt thereof:

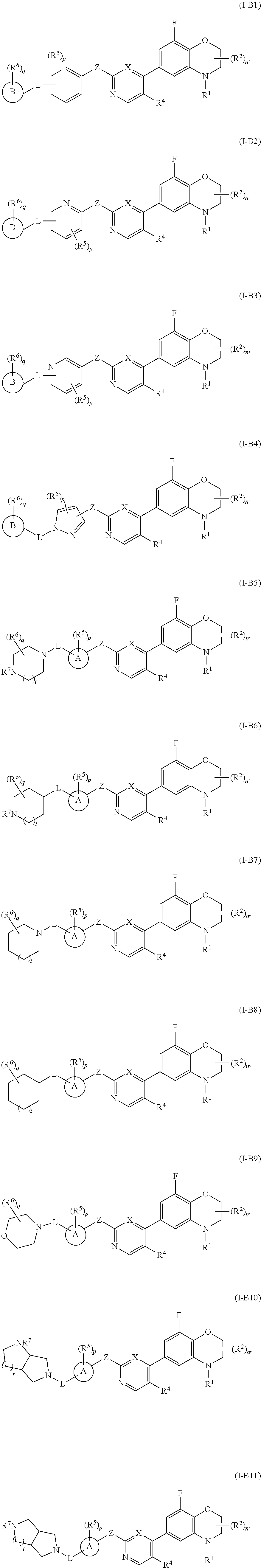

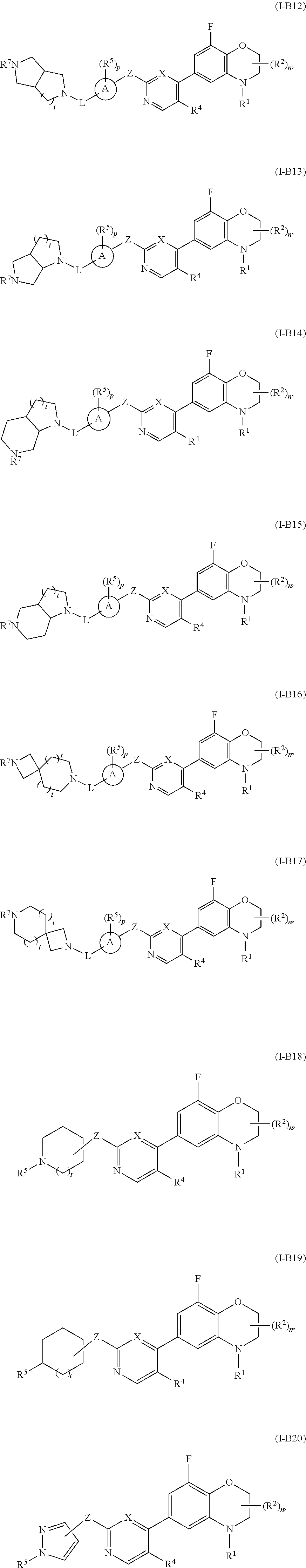

wherein X, Z, A, B, L, R¹, R², R⁴, R⁵, R⁶, R⁷, n, p, and q are as described herein for Formula (I) and t is 0, 1, 2 or 3. In some embodiments, t is 0. In some embodiments, t is 0 or 1. In some embodiments, t is 0, 1, or 2.

In some embodiments, provided is a compound of any one of Formula (I-C1) to (I-C45):

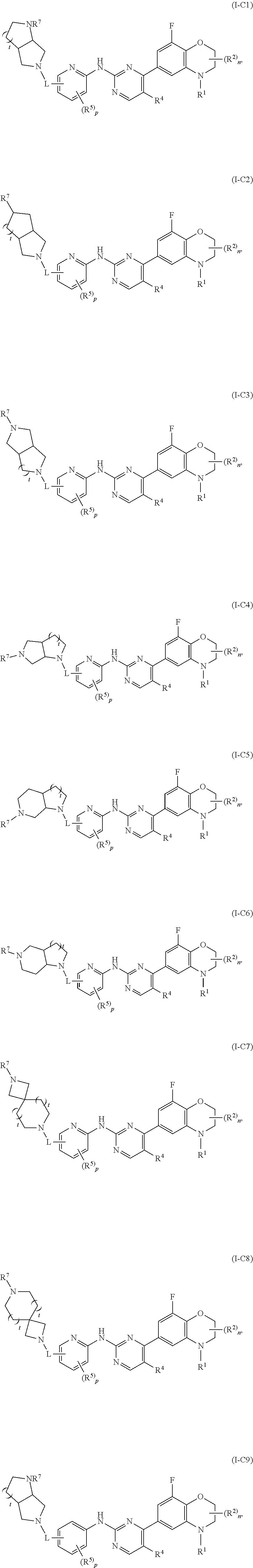

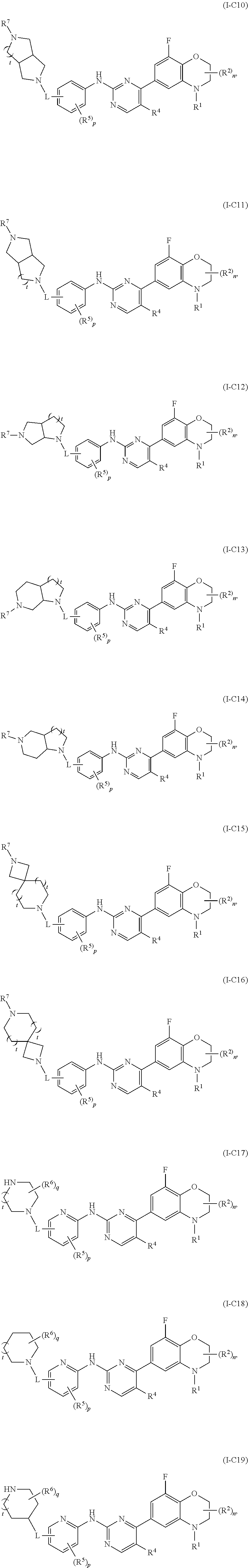

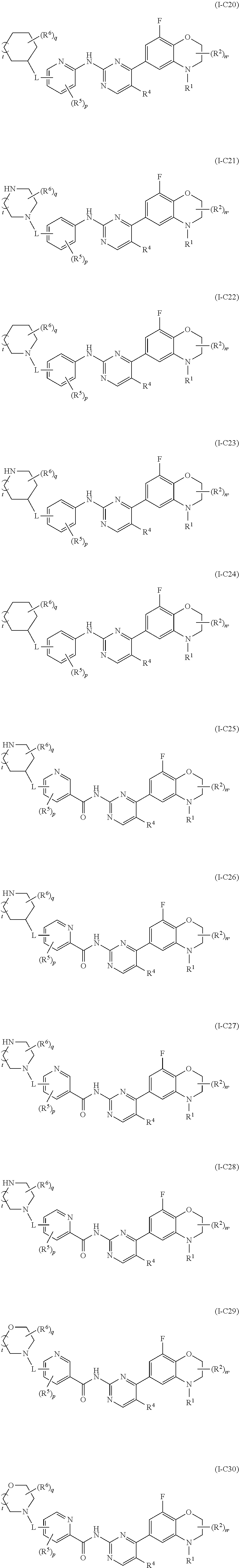

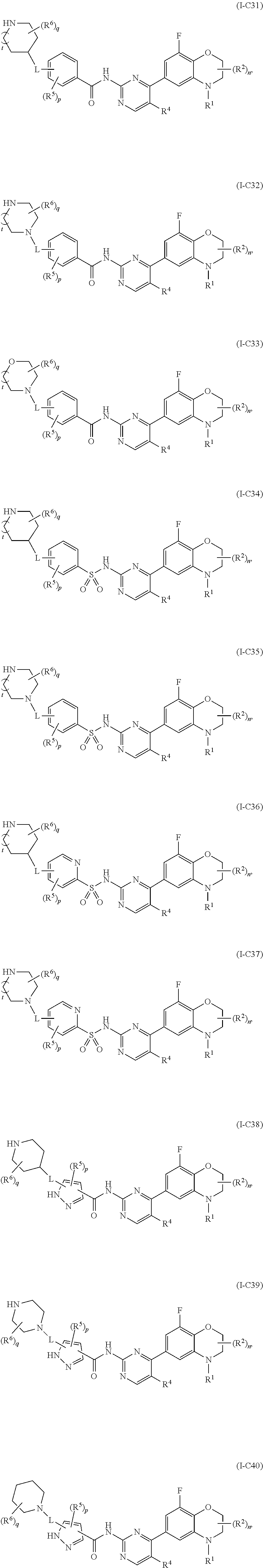

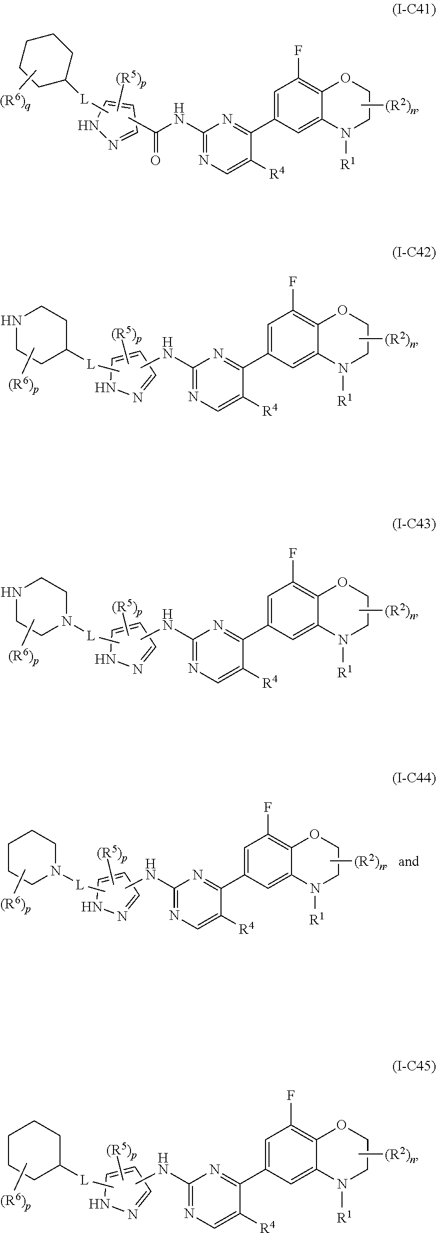

wherein L, R¹, R², R⁴. R⁵, R⁶, R⁷, n, p, and q are as described herein for Formula (I) and t is 0, 1, 2 or 3. In some embodiments, t is 0. In some embodiments, t is 0 or 1. In some embodiments, t is 0, 1, or 2.

In some embodiments of a compound of Formula (I) or a variation thereof, p is 0. In some embodiments, p is 1. In some embodiments, p is 2. In some embodiments, p is 3. In some embodiments of a compound of Formula (I), p is 0 or 1. In some embodiments, p is 0, 1, or 2.

In some embodiments of a compound of Formula (I) or a variation thereof, each R⁵ is independently C₁-C₆ alkyl, halogen, oxo, —CN, —OR¹⁰, —NR¹¹R¹², —S(O)₂R¹⁰, —NR¹⁰S(O)₂R¹¹, —C(O)R¹⁰, —NR¹⁰C(O)R¹¹, —C(O)NR¹¹R¹², C₃-C₆ cycloalkyl, —(C₁-C₃ alkylene)OR¹⁰, —(C₁-C₃ alkylene)NR¹¹R¹², —(C₁-C₃ alkylene)C(O)R¹⁰, —(C₁-C₃ alkylene)(C₃-C₆ cycloalkyl), or —(C₁-C₃ alkylene)(3- to 12-membered heterocyclyl), wherein the C₁-C₆ alkyl, —OR¹⁰, —NR¹¹R¹², —S(O)₂R¹⁰, —NR¹⁰S(O)₂R¹¹, —C(O)R¹⁰. —NR¹⁰C(O)R¹¹, —C(O)NR¹¹R¹², C₃-C₆ cycloalkyl. —(C₁-C₃ alkylene)OR¹⁰, —(C₁-C₃ alkylene)NR¹¹R¹², —(C₁-C₃ alkylene)C(O)R¹⁰, —(C₁-C₃ alkylene)(C₃-C₆ cycloalkyl), and —(C₁-C₃ alkylene)(3- to 12-membered heterocyclyl) of R⁵ are each independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen. In some embodiments, each R⁵ is independently C₁-C₆ alkyl, halogen, —CN, —OR¹⁰, —NR¹¹R¹². —S(O)₂R¹⁰, —NR¹⁰S(O)₂R¹¹, —C(O)R¹⁰, —NR¹⁰C(O)R¹¹, or —C(O)NR¹¹R¹², wherein the C₁-C₆ alkyl, —OR¹⁰, —NR¹¹R¹², —S(O)₂R¹⁰, —NR¹⁰S(O)₂R¹⁰, —C(O)R¹⁰, —NR¹⁰C(O)R¹¹, and —C(O)NR¹¹R¹² of R⁵ are each independently optionally substituted by halogen, —OR¹³ or —NR¹³R¹⁴. In some embodiments, each R⁵ is independently —CH₃, —S(O)₂CH₃, —CH₂CH₂OCH₃, —CH₂CH₂N(CH₃)₂, —NH₂, —NHS(O)₂CH₃, —N(CH₃)₂, —NHC(O)CH₂OH, —C(O)CH₂OH, C₁, —CF₃, —CN, —CH₂OH, or —C(O)NH₂.

In some embodiments of a compound of Formula (I) or a variation thereof, q is 0. In some embodiments, q is 1. In some embodiments, q is 2. In some embodiments, q is 3. In some embodiments of a compound of Formula (I), q is 0 or 1. In some embodiments, q is 0, 1, or 2.

In some embodiments of a compound of Formula (I) or a variation thereof, each R⁶ is independently C₁-C₆ alkyl, halogen, oxo, —NR¹¹R¹², —C(O)R¹⁰, C₃-C₆ cycloalkyl, 3- to 6-membered heterocyclyl, —(C₁-C₃ alkylene)OR¹⁰, or —(C₁-C₃ alkylene)NR¹¹R¹², wherein the C₁-C₆ alkyl. —NR¹¹R¹², —C(O)R¹⁰, C₃-C₆ cycloalkyl, 3- to 6-membered heterocyclyl. —(C₁-C₃alkylene)OR¹⁰, and —(C₁-C₃ alkylene)NR¹¹R¹² of R⁶ are each independently optionally substituted by halogen, oxo, —OR¹³, —NR¹³R¹¹, —C(O)R¹³, —CN, —(C₁-C₃ alkylene)OR¹³, —(C₁-C₃alkylene)NR¹³R¹⁴, —(C₁-C₃ alkylene)C(O)R¹³, C₃-C₈ cycloalkyl, or C₁-C₆ alkyl optionally substituted by oxo, —OH or halogen. In some embodiments, each R⁶ is independently C₁-C₆ alkyl, —OR¹⁰, 3- to 6-membered heterocyclyl, or —NR¹¹R¹², wherein the C₁-C₆ alkyl, —OR¹⁰, 3- to 6-membered heterocyclyl, and —NR¹¹R¹² of R⁶ are each independently optionally substituted by —OR¹³. In some embodiments, each R⁶ is independently —CH₃, —CH₂CH₃, —CH₂OH, —OH. —NH₂, oxetanyl, or —N(CH₃)₂.

In some embodiments of a compound of Formula (I) or a variation thereof, A, L, and B together with R⁵ and R⁶ form a moiety selected from the group consisting of:

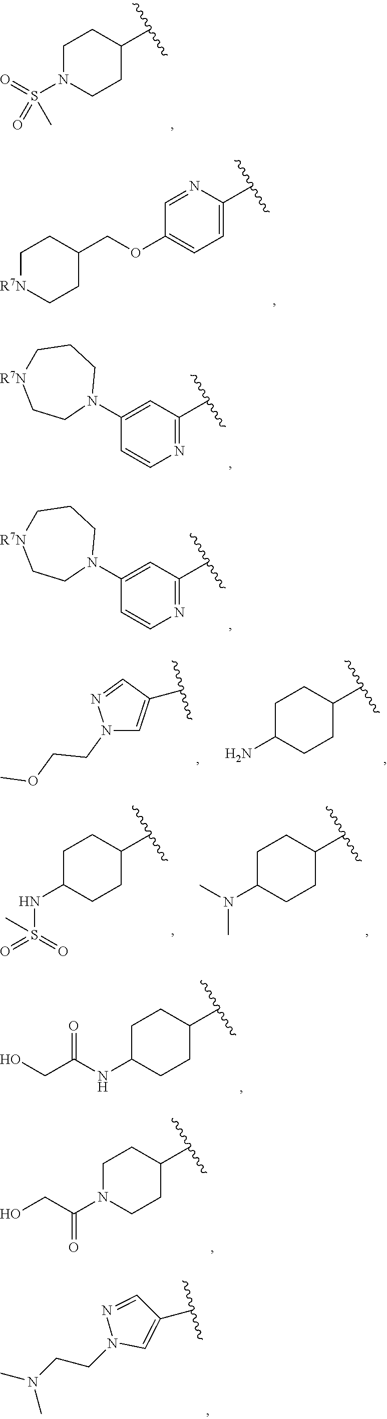

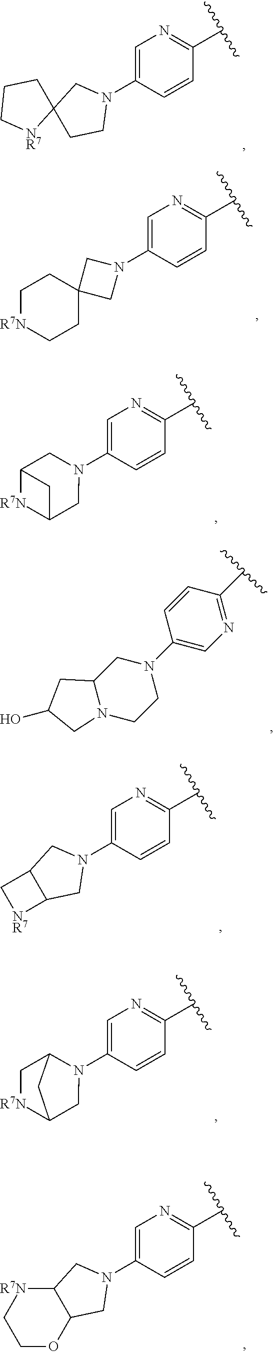

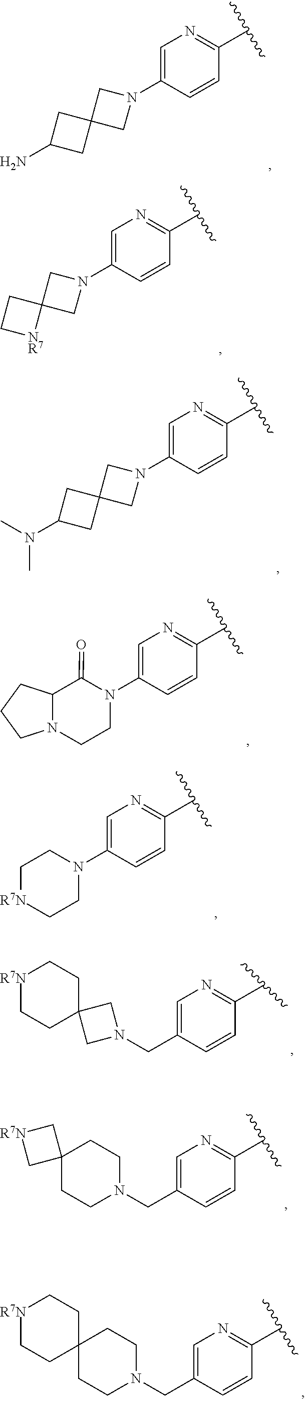

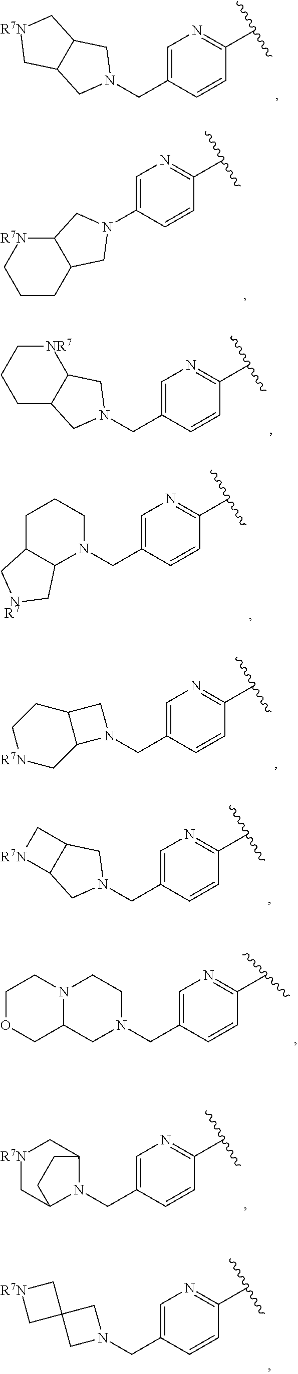

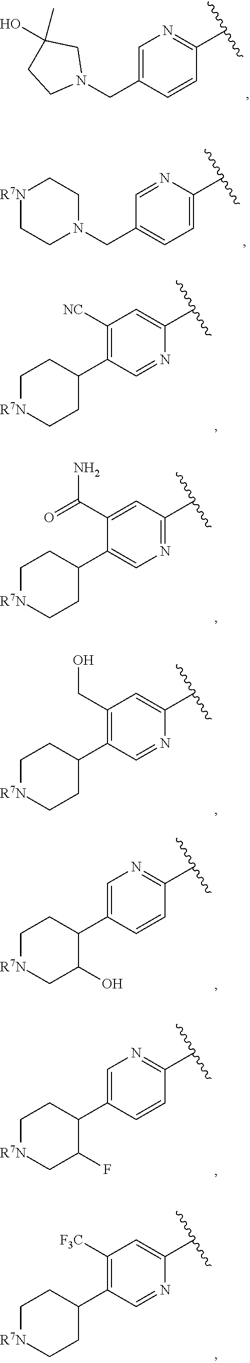

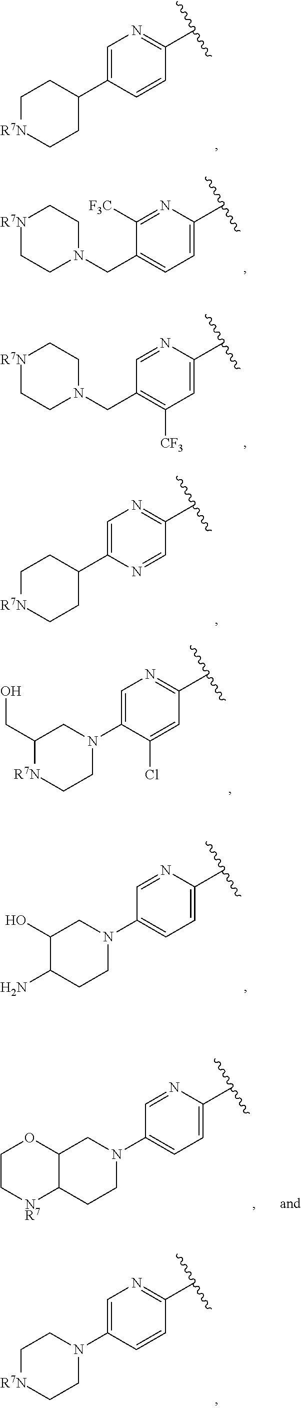

wherein the wavy lines denote attachment points to the parent molecule and R⁷ is as defined herein for Formula (I).

In some embodiments of a compound of Formula (I) or a variation thereof, R⁷ is hydrogen, C₁-C₆ alkyl, C₃-C₆ cycloalkyl, or —C(O)R¹⁰, wherein the C₁-C₆ alkyl, C₃-C₆ cycloalkyl, and —C(O)R¹⁰ of R are each independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₅ cycloalkyl, or C₁-C₆ alkyl optionally substituted by oxo, —OH or halogen. In some embodiments, R⁷ is hydrogen or C₁-C₆ alkyl. In some embodiments, R⁷ is hydrogen. In some embodiments, R⁷ is C₁-C₆ alkyl. In some embodiments, R⁷ is methyl.

In some embodiments of a compound of Formula (I) or a variation thereof, X is N. In some embodiments, X is CR^{a}. In some embodiments, X is CR^{a}, wherein R^{a} is hydrogen. In some embodiments, X is CR^{a}, wherein R^{a} is —CN.

In some embodiments of a compound of Formula (I) or a variation thereof, R¹ is C₁-C₆ alkyl, C₂-C₆ alkenyl, C₂-C₆ alkynyl, C₃-C₆ cycloalkyl, 3- to 12-membered heterocyclyl, —(C₁-C₃ alkylene)(C₃-C₆ cycloalkyl), or —(C₁-C₃ alkylene)(3- to 12-membered heterocyclyl), each of which is optionally substituted by halogen, —OR¹³, —C(O)NR¹³R¹⁴, —NR¹³R¹⁴, —C(O)R¹³, —CN, C₃-C₈ cycloalkyl, or C₁-C₆ alkyl optionally substituted by oxo. —OH or halogen. In some embodiments, R¹ is C₁-C₆ alkyl. C₃-C₆ cycloalkyl, 3- to 12-membered heterocyclyl, —(C₁-C₃ alkylene)(C₃-C₆ cycloalkyl), —(C₁-C₃ alkylene)(3- to 12-membered heterocyclyl), each of which is unsubstituted. In some embodiments, R¹ is C₁-C₆ alkyl, C₃-C₆ cycloalkyl, 3- to 12-membered heterocyclyl, or —(C₁-C₃ alkylene)(C₃-C₆ cycloalkyl), each of which is independently optionally substituted by halogen, —OR¹³, —NR¹³R¹⁴, —C(O)R¹³, —CN, C₃-C₈ cycloalkyl, or C₁-C₆ alkyl optionally substituted by oxo, —OH or halogen. In some embodiments, R¹ is C₁-C₆ alkyl or C₃-C₆ cycloalkyl, each of which is independently optionally substituted by halogen, —OR¹³, or C₁-C₆ alkyl. In some embodiments, R¹ is C₁-C₆ alkyl or C₃-C₆ cycloalkyl, each of which is independently optionally substituted by halogen, —OH, or C₁-C₆ alkyl. In some embodiments, R¹ is C₁-C₆ alkyl optionally substituted by halogen or —OR¹³. In some embodiments, R¹ is C₃-C₆ cycloalkyl optionally substituted by halogen. —OR¹³, or C₁-C₆ alkyl.

In some embodiments of a compound of Formula (I) or a variation thereof, R¹ is selected from the group consisting of

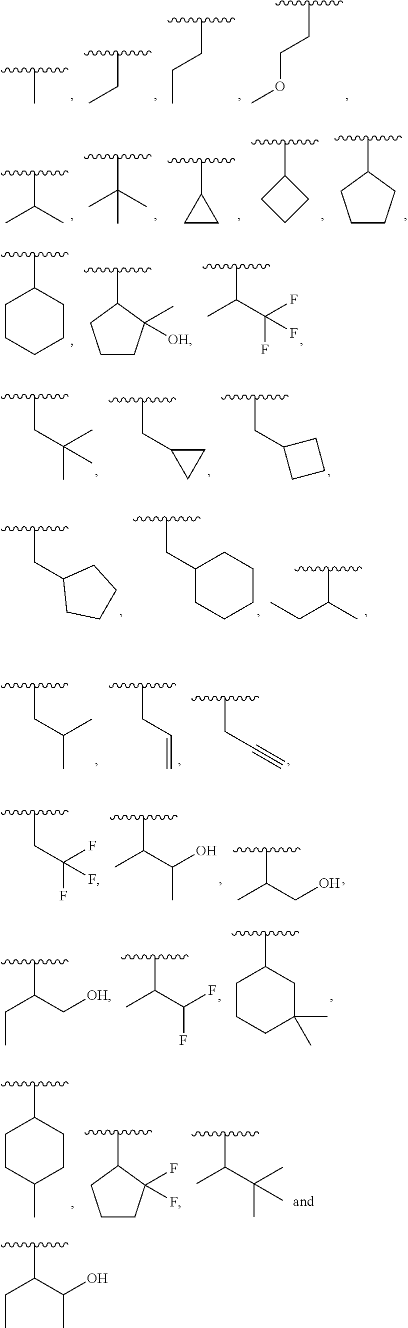

wherein the wavy lines denote attachment points to the parent molecule.

In some embodiments. R¹ is

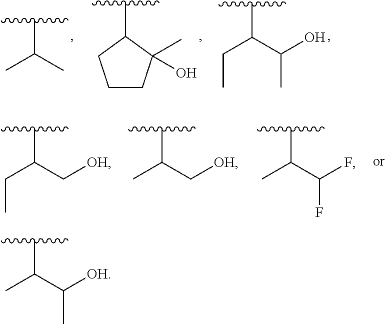

In some embodiments, R¹ is

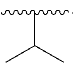 [SMILES: *C(C)C]

In some embodiments of a compound of Formula (I) or a variation thereof, n is 0. In some embodiments, n is 1. In some embodiments, n is 2. In some embodiments, n is 0 or 1. In some embodiments, n is 0, 1, or 2. In some embodiments, n is 0, 1, 2, or 3.

In some embodiments of a compound of Formula (I) or a variation thereof, each R² is independently C₁-C₆ alkyl, oxo, —NR¹¹R¹², —CN, or halogen. In some embodiments, each R² is independently C₁-C₆ alkyl, oxo, or halogen. In some embodiments, each R² is independently C₁-C₆ alkyl or halogen. In some embodiments. R² is oxo. In some embodiments, each R² is independently —NR¹¹R¹². In some embodiments, R² is —CN. In some embodiments, each R² is independently —C(O)R¹⁰. In some embodiments, each R² is independently —C(O)NR¹¹R¹². In some embodiments, each R² is independently halogen, such as fluoro or chloro. In some embodiments, each R² is independently C₁-C₆ alkyl, such as methyl or dimethyl attached to the same carbon. In some embodiments, groups of R² (such as when more than one R² is present) are oxo and methyl, independently attached to two different carbons. In some embodiments, groups of R² are oxo and dimethyl, independently attached to two different carbons. In some embodiments, groups of R² are oxo and —CN, independently attached to two different carbons. In some embodiments, groups of R² are oxo and —NR¹¹R¹², independently attached to two different carbons. In some embodiments, groups of R² are oxo and —C(O)R¹⁰, independently attached to two different carbons. In some embodiments, groups of R² are oxo and —C(O)NR¹¹R¹², independently attached to two different carbons. In some embodiments, groups of R² are difluoro attached to the same carbon. In some embodiments, groups of R² are dichloro attached to the same carbon. In some embodiments, groups of R² are oxo and fluoro or difluoro, each independently attached to two different carbons. In some embodiments, n is 0, 1, or 2; and each R² is independently C₁-C₆ alkyl or halogen.

In some embodiments of a compound of Formula (I) or a variation thereof, R⁴ is hydrogen. C₁-C₆ alkyl, C₃-C₆ cycloalkyl. C₁-C₆ haloalkyl, C₁-C₆ alkoxy, C₁-C₆ haloalkoxy, halogen, —CN or —OH. In some embodiments, R⁴ is hydrogen. In some embodiments, R⁴ is C₁-C₆alkyl. In some embodiments. R⁴ is C₃-C₆ cycloalkyl. In some embodiments, R⁴ is C₁-C₆ haloalkyl. In some embodiments, R⁴ is C₁-C₆ alkoxy. In some embodiments, R⁴ is C₁-C₆ haloalkoxy. In some embodiments, R⁴ is halogen. In some embodiments. R⁴ is —CN. In some embodiments, R⁴ is —OH. In some embodiments, R⁴ is independently C₁-C₆ alkyl, C₃-C₆ cycloalkyl, C₁-C₆ haloalkyl, C₁-C₆ alkoxy, C₁-C₆ haloalkoxy or halogen. In some embodiments, R⁴ is fluoro, chloro, methyl, trifluoromethyl, trifluoromethoxy, methoxy, or cyclopropyl. In some embodiments. R⁴ is halogen. In some embodiments, R⁴ is fluoro.

In some embodiments of a compound of Formula (I) or a variation thereof, X is CR^{a}, wherein R^{a} is hydrogen; and R⁴ is hydrogen, C₁-C₆ alkyl, C₃-C₆ cycloalkyl, C₁-C₆ haloalkyl, C₁-C₆ alkoxy, C₁-C₆ haloalkoxy, halogen, —CN, or —OH. In some embodiments; X is N; and R⁴ is hydrogen, C₁-C₆ alkyl, C₃-C₆ cycloalkyl, C₁-C₆ haloalkyl, C₁-C₆ alkoxy, C₁-C₆ haloalkoxy, halogen, —CN or —OH. In some embodiments, X is CR^{a}, wherein R¹ is —CN; and R⁴ is hydrogen, C₁-C₆ alkyl, C₃-C₆ cycloalkyl. C₁-C₆ haloalkyl, C₁-C₆ alkoxy. C₁-C₆ haloalkoxy, halogen, —CN or —OH. In some embodiments, X is CR^{a}, wherein R^{a} is hydrogen; and R⁴ is halogen. In some embodiments; X is N; and R⁴ is halogen. In some embodiments, X is CR^{a}, wherein R^{a} is —CN; and R⁴ is halogen.

In some embodiments of a compound of Formula (I) or a variation thereof, X is CR^{a}, wherein R^{a} is hydrogen; and R⁴ is F. In some embodiments, X is CR^{a}, wherein R^{a} is —CN; and R⁴ is F. In some embodiments, X is N; and R⁴ is F. In some embodiments, X is N; and R⁴ is Cl. In some embodiments of a compound of Formula (I), X is CR^{a}, wherein R¹ is hydrogen; and R⁴ is Cl. In some embodiments of a compound of Formula (I). X is CR^{a}, wherein R^{a} is —CN; and R⁴ is Cl.

In some embodiments of a compound of Formula (I) or a variation thereof. X is CR^{a}, wherein R^{a} is hydrogen; R⁴ is F; and each R² is independently hydrogen. C₁-C₆ alkyl, oxo, —NR¹¹R¹², —CN, —C(O)R¹⁰, —C(O)NR¹¹R¹², or halogen. In some embodiments, X is N; R⁴ is F; and each R² is independently C₁-C₆alkyl, oxo, —NR¹¹R¹², —CN. —C(O)R¹⁰, —C(O)NR¹¹R¹² or halogen. In some embodiments, X is N; R⁴ is F; and each R² is F, wherein each F of R² is attached to same carbon or two different carbons. In some embodiments, X is N; R⁴ is F; and each R² is halogen, wherein each halogen is attached to same carbon or two different carbons. In some embodiments. X is N; R⁴ is F; and each R² is independently C₁-C₆ alkyl. In some embodiments. X is N; R⁴ is F; and each R² is oxo or methyl, attached to two different carbons. In some embodiments. X is N; R⁴ is F; and each R² is oxo or F, which are attached to two different carbons. In some embodiments. X is N; R⁴ is F; R² is oxo. In some embodiments, X is N; R⁴ is F; and n is 0. In some embodiments, X is N; R⁴ is F; and each R² is independently C₁-C₆ alkyl or halogen.

In some embodiments of a compound of Formula (I) or a variation thereof, X is N; R⁴ is hydrogen. C₁-C₆ alkyl, C₃-C₆ cycloalkyl, C₁-C₆ haloalkyl, C₁-C₆ alkoxy, C₁-C₆ haloalkoxy, halogen, —CN, or OH; each R² is independently C₁-C₆alkyl, oxo, —NR¹¹R¹². —CN. —C(O)R¹⁰, —C(O)NR¹¹R¹² or halogen, any two R² groups are independently attached to same carbon or two different carbons; and R¹ is C₁-C₆ alkyl, C₂-C₆ alkenyl, C₂-C₆ alkynyl, C₁-C₅alkoxy, C₃-C₆ cycloalkyl, 3- to 12-membered heterocyclyl, —(C₁-C₃ alkylene)(C₃-C₆ cycloalkyl), or —(C₁-C₃ alkylene)(3- to 12-membered heterocyclyl), each of which is optionally substituted by halogen, —OR¹³, —C(O) NR¹³R¹⁴, —NR¹³R¹⁴, —C(O)R¹³, —CN, C₃-C₈ cycloalkyl, or C₁-C₆ alkyl optionally substituted by oxo, —OH or halogen.

In some embodiments of a compound of Formula (I) or a variation thereof, X is CH; R⁴ is C₁-C₆ alkyl, C₃-C₆ cycloalkyl, C₁-C₆ haloalkyl, C₁-C₆ alkoxy, C₁-C₆ haloalkoxy, halogen, or —OH; each R² is independently C₁-C₆ alkyl, oxo, —NR¹¹R¹², —CN, —C(O)R¹⁰, —C(O)NR¹¹R¹² or halogen, any two R² groups are independently attached to same carbon or two different carbons; R¹ is C₁-C₆ alkyl, C₃-C₆ cycloalkyl, 3- to 12-membered heterocyclyl, —(C₁-C₃ alkylene)(C₃-C₆ cycloalkyl), —(C₁-C₃ alkylene)(3- to 12-membered heterocyclyl), each of which is optionally substituted by halogen, —OR³, —C(O) NR¹³R¹⁴, —NR¹³R¹⁴, —C(O)R¹³, —CN, C₃-C₈ cycloalkyl, or C₁-C₆ alkyl optionally substituted by oxo, —OH or halogen.

In some embodiments of a compound of Formula (I) or a variation thereof, X is N; R⁴ is F; each R² is independently C₁-C₆alkyl, oxo, —NR¹¹R¹², —CN, —C(O)R¹⁰, —C(O)NR¹¹R¹² or halogen, any two R² groups are independently attached to same carbon or two different carbons; R¹ is C₁-C₆alkyl, C₃-C₆ cycloalkyl, 3- to 12-membered heterocyclyl. —(C₁-C₃ alkylene)(C₃-C₆ cycloalkyl), —(C₁-C₃ alkylene)(3- to 12-membered heterocyclyl), each of which is optionally substituted by halogen, —OR¹³, —C(O)NR¹³R¹⁴, —NR¹³R¹⁴, —C(O)R¹³, —CN, C₃-C₈ cycloalkyl, or C₁-C₆ alkyl optionally substituted by oxo, —OH or halogen. In some embodiments, X is N; R⁴ is F; each R² is independently C₁-C₆ alkyl or halogen, any two R² groups are independently attached to same carbon or two different carbons; R¹ is C₁-C₆ alkyl or C₃-C₆ cycloalkyl, each of which is independently optionally substituted by halogen, —OH, or C₁-C₆ alkyl.

In some embodiments of a compound of Formula (I) or a variation thereof, X is N; R⁴ is F; each R² is independently C₁-C₆alkyl, any two R² groups are independently attached to same carbon or two different carbons; and R¹ is C₁-C₆alkyl, C₃-C₆ cycloalkyl, 3- to 12-membered heterocyclyl, —(C₁-C₃ alkylene)(C₃-C₆ cycloalkyl), —(C₁-C₃ alkylene)(3- to 12-membered heterocyclyl), each of which is optionally substituted by halogen, —OR¹³—C(O) NR¹³R¹⁴, —NR¹³R¹⁴, —C(O)R¹³, —CN, C₃-C₈ cycloalkyl, or C₁-C₆ alkyl optionally substituted by oxo, —OH or halogen. In some embodiments, X is N; R⁴ is F; each R² is C₁-C₆ alkyl, any two R² groups are independently attached to same carbon or two different carbons; R¹ is C₁-C₆alkyl or C₃-C₆ cycloalkyl, each of which is independently optionally substituted by halogen, —OH, or C₁-C₆ alkyl.

In some embodiments of a compound of Formula (I) or a variation thereof, X is N, R⁴ is F; n is 0; and R¹ is C₁-C₆ alkyl or C₃-C₆ cycloalkyl, wherein R¹ is independently optionally substituted by halogen. —OR¹³—NR¹³R¹⁴ or C₁-C₆ alkyl optionally substituted by oxo, —OH or halogen. In some embodiments, X is N; R⁴ is F; n is 0; and R¹ is C₁-C₆ alkyl or C₃-C₆ cycloalkyl, each of which is independently optionally substituted by halogen, —OH, or C₁-C₆ alkyl. In some embodiments, X is N; R⁴ is F; n is 0; R¹ is selected from the group consisting of:

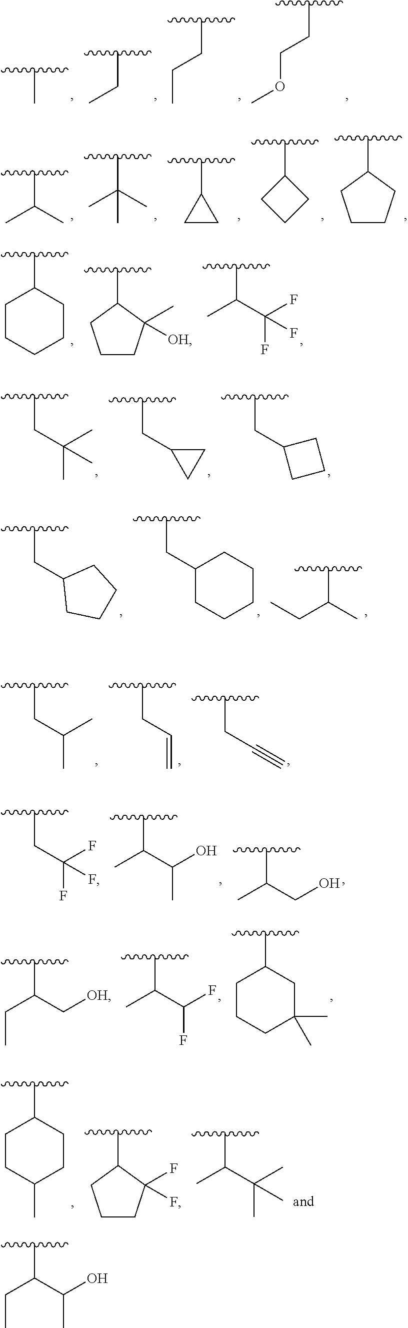

wherein the wavy lines denote attachment points to the parent molecule. In some embodiments of a compound of Formula (I). X is N, R⁴ is F; n is 0; R¹ is C₁-C₆ alkyl.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to A of Formula (I) may be combined with every description, variation, embodiment or aspect of B, X, Z, R², R⁴, R⁵, R⁶, m, n, p, and q the same as if each and every combination were specifically and individually listed. It is also understood that all descriptions, variations, embodiments or aspects of Formula (I), where applicable, apply equally to other formulae detailed herein, and are equally described, the same as if each and every description, variation, embodiment or aspect were separately and individually listed for all formulae. For example, it is understood that, all descriptions, variations, embodiments or aspects of Formula (I), where applicable, apply equally to any of related formulae as detailed herein, such as Formulae (I-A), (I-B1) to (I-B20), (I-C1) to (I-C45), and are equally described, the same as if each and every description, variation, embodiment or aspect were separately and individually listed for all formulae.

Also provided are salts of compounds referred to herein, such as pharmaceutically acceptable salts. The invention also includes any or all of the stereochemical forms, including any enantiomeric or diastereomeric forms, and any tautomers or other forms of the compounds described. It is understood that individual enantiomers and diastereomers are provided herein and their corresponding structures can be readily determined.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds are listed in Table 1.

TABLE 1

| Com- | |
|---|---|
| pound | |
| No. | Structure |
| 1 | 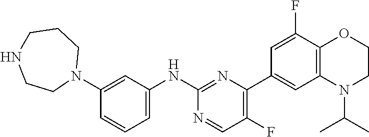 [SMILES: CC(C)N1CCOc2c(F)cc(-c3nc(Nc4cccc(N5CCCNCC5)c4)ncc3F)cc21] |
| 2 | 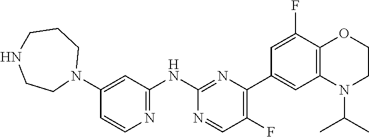 [SMILES: CC(C)N1CCOc2c(F)cc(-c3nc(Nc4cc(N5CCCNCC5)ccn4)ncc3F)cc21] |
| 3 | 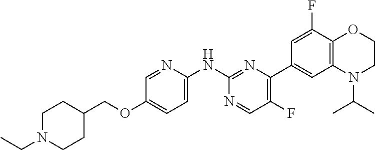 [SMILES: CCN1CCC(COc2ccc(Nc3ncc(F)c(-c4cc(F)c5c(c4)N(C(C)C)CCO5)n3)nc2)CC1] |
| 4 | 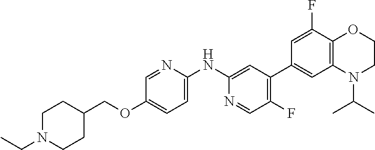 [SMILES: CCN1CCC(COc2ccc(Nc3cc(-c4cc(F)c5c(c4)N(C(C)C)CCO5)c(F)cn3)nc2)CC1] |
| 5 | 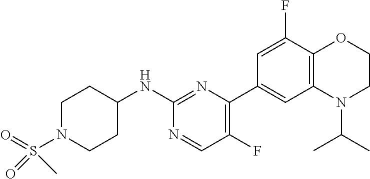 [SMILES: CC(C)N1CCOc2c(F)cc(-c3nc(NC4CCN(S(C)(=O)=O)CC4)ncc3F)cc21] |
| 6 | 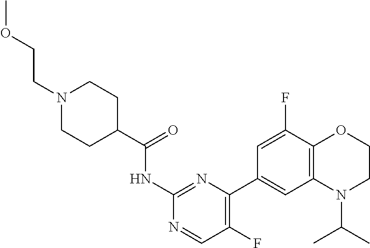 [SMILES: COCCN1CCC(C(=O)Nc2ncc(F)c(-c3cc(F)c4c(c3)N(C(C)C)CCO4)n2)CC1] |
| 7 | 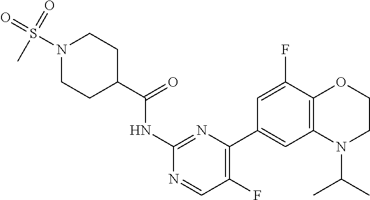 [SMILES: CC(C)N1CCOc2c(F)cc(-c3nc(NC(=O)C4CCN(S(C)(=O)=O)CC4)ncc3F)cc21] |
| 8 | 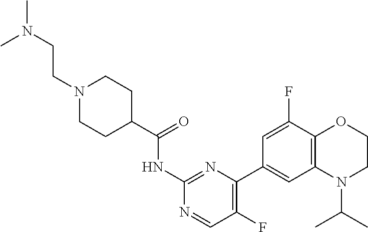 [SMILES: CC(C)N1CCOc2c(F)cc(-c3nc(NC(=O)C4CCN(CCN(C)C)CC4)ncc3F)cc21] |
| 9 | 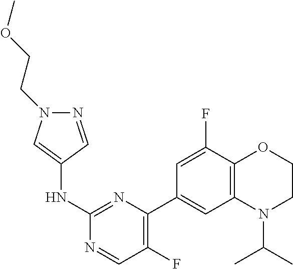 [SMILES: COCCn1cc(Nc2ncc(F)c(-c3cc(F)c4c(c3)N(C(C)C)CCO4)n2)cn1] |
| 10 | 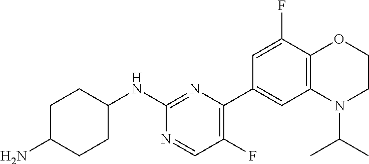 [SMILES: CC(C)N1CCOc2c(F)cc(-c3nc(NC4CCC(N)CC4)ncc3F)cc21] |
| 11 | 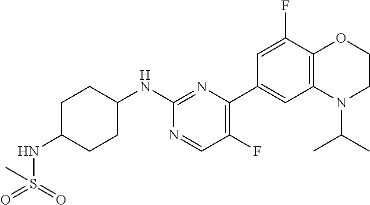 [SMILES: CC(C)N1CCOc2c(F)cc(-c3nc(NC4CCC(NS(C)(=O)=O)CC4)ncc3F)cc21] |
| 12 | 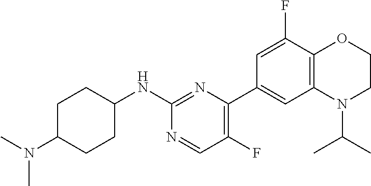 [SMILES: CC(C)N1CCOc2c(F)cc(-c3nc(NC4CCC(N(C)C)CC4)ncc3F)cc21] |
| 13 | 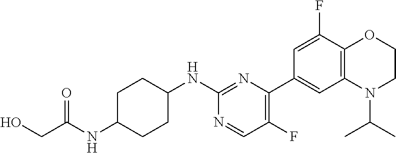 [SMILES: CC(C)N1CCOc2c(F)cc(-c3nc(NC4CCC(NC(=O)CO)CC4)ncc3F)cc21] |
| 14 | 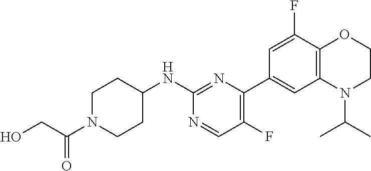 [SMILES: CC(C)N1CCOc2c(F)cc(-c3nc(NC4CCN(C(=O)CO)CC4)ncc3F)cc21] |
| 15 | 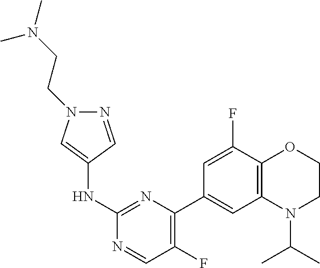 [SMILES: CC(C)N1CCOc2c(F)cc(-c3nc(Nc4cnn(CCN(C)C)c4)ncc3F)cc21] |
| 16 | 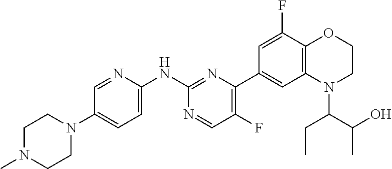 [SMILES: CCC(C(C)O)N1CCOc2c(F)cc(-c3nc(Nc4ccc(N5CCN(C)CC5)cn4)ncc3F)cc21] |
| 17 | 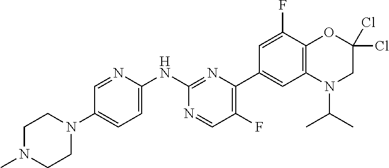 [SMILES: CC(C)N1CC(Cl)(Cl)Oc2c(F)cc(-c3nc(Nc4ccc(N5CCN(C)CC5)cn4)ncc3F)cc21] |
| 18 | 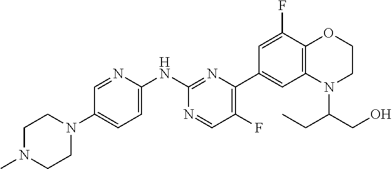 [SMILES: CCC(CO)N1CCOc2c(F)cc(-c3nc(Nc4ccc(N5CCN(C)CC5)cn4)ncc3F)cc21] |
| 19 | 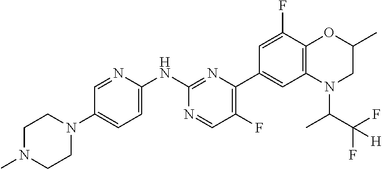 [SMILES: CC1CN(C(C)C(F)F)c2cc(-c3nc(Nc4ccc(N5CCN(C)CC5)cn4)ncc3F)cc(F)c2O1] |
| 20 | 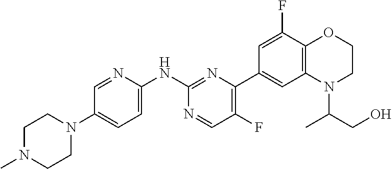 [SMILES: CC(CO)N1CCOc2c(F)cc(-c3nc(Nc4ccc(N5CCN(C)CC5)cn4)ncc3F)cc21] |
| 21 | 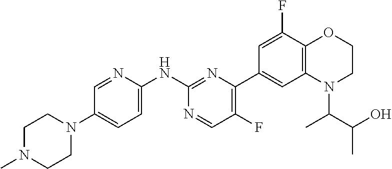 [SMILES: CC(O)C(C)N1CCOc2c(F)cc(-c3nc(Nc4ccc(N5CCN(C)CC5)cn4)ncc3F)cc21] |
| 22 | 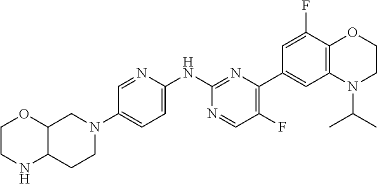 [SMILES: CC(C)N1CCOc2c(F)cc(-c3nc(Nc4ccc(N5CCC6NCCOC6C5)cn4)ncc3F)cc21] |
| 23 | 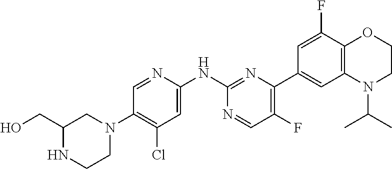 [SMILES: CC(C)N1CCOc2c(F)cc(-c3nc(Nc4cc(Cl)c(N5CCNC(CO)C5)cn4)ncc3F)cc21] |
| 24 | 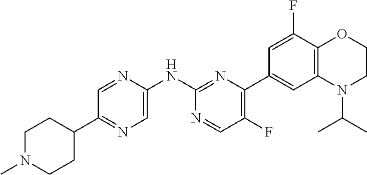 [SMILES: CC(C)N1CCOc2c(F)cc(-c3nc(Nc4cnc(C5CCN(C)CC5)cn4)ncc3F)cc21] |
| 25 | 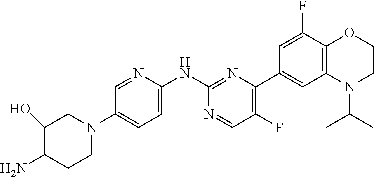 [SMILES: CC(C)N1CCOc2c(F)cc(-c3nc(Nc4ccc(N5CCC(N)C(O)C5)cn4)ncc3F)cc21] |
| 26 | 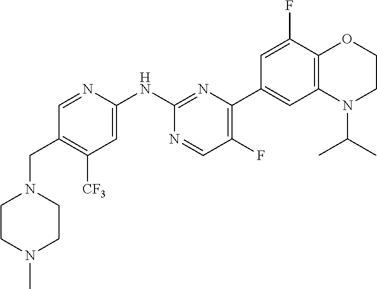 [SMILES: CC(C)N1CCOc2c(F)cc(-c3nc(Nc4cc(C(F)(F)F)c(CN5CCN(C)CC5)cn4)ncc3F)cc21] |
| 27 | 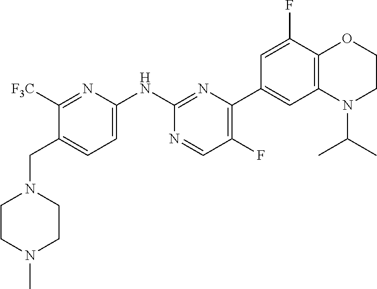 [SMILES: CC(C)N1CCOc2c(F)cc(-c3nc(Nc4ccc(CN5CCN(C)CC5)c(C(F)(F)F)n4)ncc3F)cc21] |
| 28 | 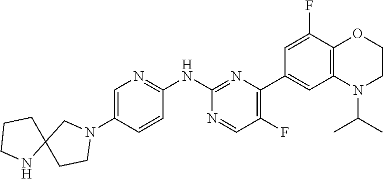 [SMILES: CC(C)N1CCOc2c(F)cc(-c3nc(Nc4ccc(N5CCC6(CCCN6)C5)cn4)ncc3F)cc21] |
| 29 | 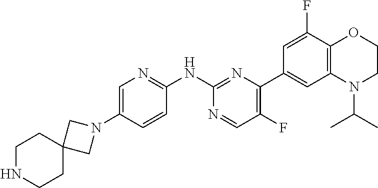 [SMILES: CC(C)N1CCOc2c(F)cc(-c3nc(Nc4ccc(N5CC6(CCNCC6)C5)cn4)ncc3F)cc21] |
| 30 | 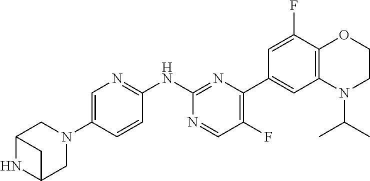 [SMILES: CC(C)N1CCOc2c(F)cc(-c3nc(Nc4ccc(N5CC6CC(C5)N6)cn4)ncc3F)cc21] |
| 31 | 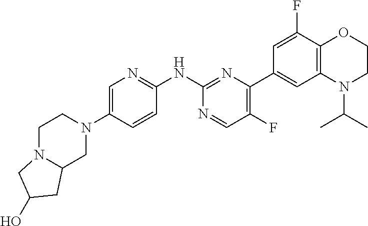 [SMILES: CC(C)N1CCOc2c(F)cc(-c3nc(Nc4ccc(N5CCN6CC(O)CC6C5)cn4)ncc3F)cc21] |
| 32 | 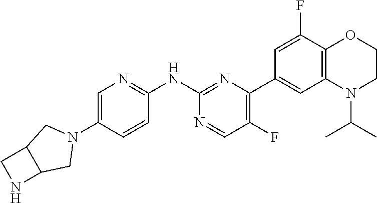 [SMILES: CC(C)N1CCOc2c(F)cc(-c3nc(Nc4ccc(N5CC6CNC6C5)cn4)ncc3F)cc21] |
| 33 | 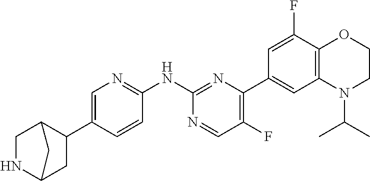 [SMILES: CC(C)N1CCOc2c(F)cc(-c3nc(Nc4ccc(C5CC6CC5CN6)cn4)ncc3F)cc21] |
| 34 | 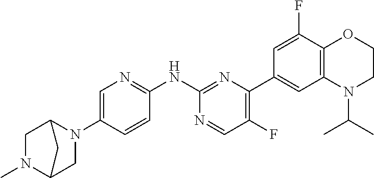 [SMILES: CC(C)N1CCOc2c(F)cc(-c3nc(Nc4ccc(N5CC6CC5CN6C)cn4)ncc3F)cc21] |
| 35 | 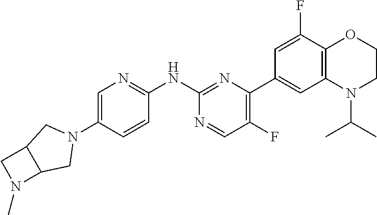 [SMILES: CC(C)N1CCOc2c(F)cc(-c3nc(Nc4ccc(N5CC6CN(C)C6C5)cn4)ncc3F)cc21] |
| 36 | 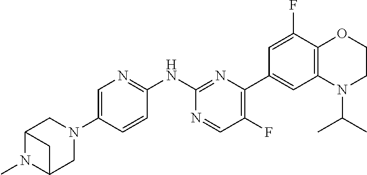 [SMILES: CC(C)N1CCOc2c(F)cc(-c3nc(Nc4ccc(N5CC6CC(C5)N6C)cn4)ncc3F)cc21] |
| 37 | 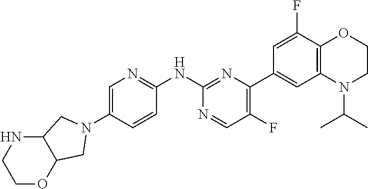 [SMILES: CC(C)N1CCOc2c(F)cc(-c3nc(Nc4ccc(N5CC6NCCOC6C5)cn4)ncc3F)cc21] |
| 38 | 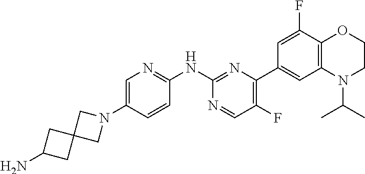 [SMILES: CC(C)N1CCOc2c(F)cc(-c3nc(Nc4ccc(N5CC6(CC(N)C6)C5)cn4)ncc3F)cc21] |
| 39 | 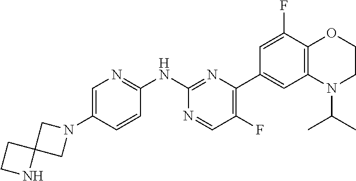 [SMILES: CC(C)N1CCOc2c(F)cc(-c3nc(Nc4ccc(N5CC6(CCN6)C5)cn4)ncc3F)cc21] |
| 40 | 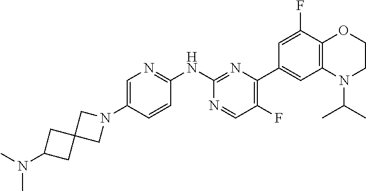 [SMILES: CC(C)N1CCOc2c(F)cc(-c3nc(Nc4ccc(N5CC6(CC(N(C)C)C6)C5)cn4)ncc3F)cc21] |
| 41 | 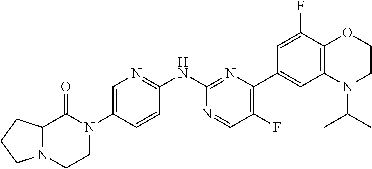 [SMILES: CC(C)N1CCOc2c(F)cc(-c3nc(Nc4ccc(N5CCN6CCCC6C5=O)cn4)ncc3F)cc21] |
| 42 | 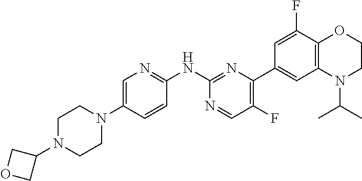 [SMILES: CC(C)N1CCOc2c(F)cc(-c3nc(Nc4ccc(N5CCN(C6COC6)CC5)cn4)ncc3F)cc21] |
| 43 | 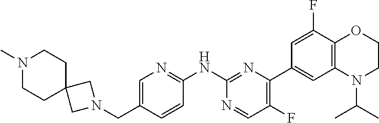 [SMILES: CC(C)N1CCOc2c(F)cc(-c3nc(Nc4ccc(CN5CC6(CCN(C)CC6)C5)cn4)ncc3F)cc21] |
| 44 | 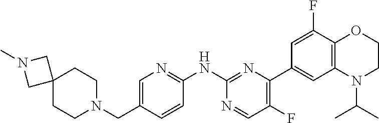 [SMILES: CC(C)N1CCOc2c(F)cc(-c3nc(Nc4ccc(CN5CCC6(CC5)CN(C)C6)cn4)ncc3F)cc21] |
| 45 | 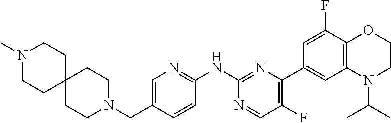 [SMILES: CC(C)N1CCOc2c(F)cc(-c3nc(Nc4ccc(CN5CCC6(CCN(C)CC6)CC5)cn4)ncc3F)cc21] |
| 46 | 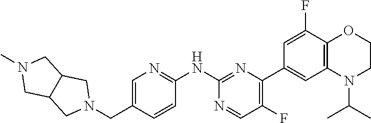 [SMILES: CC(C)N1CCOc2c(F)cc(-c3nc(Nc4ccc(CN5CC6CN(C)CC6C5)cn4)ncc3F)cc21] |
| 47 | 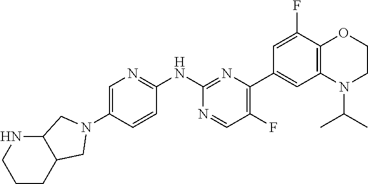 [SMILES: CC(C)N1CCOc2c(F)cc(-c3nc(Nc4ccc(N5CC6CCCNC6C5)cn4)ncc3F)cc21] |
| 48 | 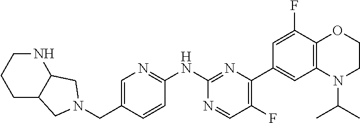 [SMILES: CC(C)N1CCOc2c(F)cc(-c3nc(Nc4ccc(CN5CC6CCCNC6C5)cn4)ncc3F)cc21] |
| 49 | 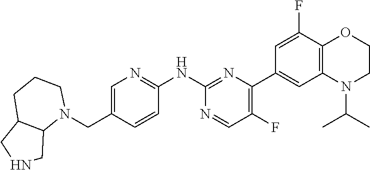 [SMILES: CC(C)N1CCOc2c(F)cc(-c3nc(Nc4ccc(CN5CCCC6CNCC65)cn4)ncc3F)cc21] |
| 50 | 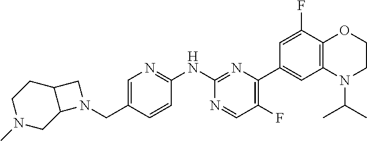 [SMILES: CC(C)N1CCOc2c(F)cc(-c3nc(Nc4ccc(CN5CC6CCN(C)CC65)cn4)ncc3F)cc21] |
| 51 | 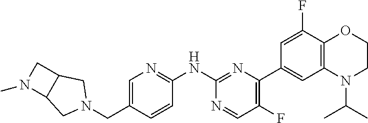 [SMILES: CC(C)N1CCOc2c(F)cc(-c3nc(Nc4ccc(CN5CC6CN(C)C6C5)cn4)ncc3F)cc21] |
| 52 | 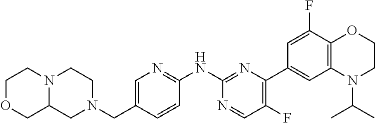 [SMILES: CC(C)N1CCOc2c(F)cc(-c3nc(Nc4ccc(CN5CCN6CCOCC6C5)cn4)ncc3F)cc21] |
| 53 | 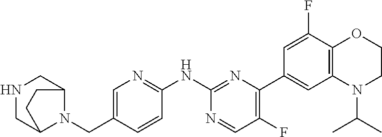 [SMILES: CC(C)N1CCOc2c(F)cc(-c3nc(Nc4ccc(CN5C6CCC5CNC6)cn4)ncc3F)cc21] |
| 54 | 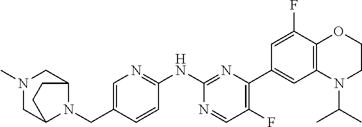 [SMILES: CC(C)N1CCOc2c(F)cc(-c3nc(Nc4ccc(CN5C6CCC5CN(C)C6)cn4)ncc3F)cc21] |
| 55 | 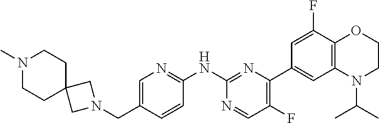 [SMILES: CC(C)N1CCOc2c(F)cc(-c3nc(Nc4ccc(CN5CC6(CCN(C)CC6)C5)cn4)ncc3F)cc21] |
| 56 | 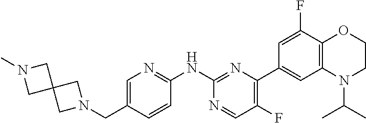 [SMILES: CC(C)N1CCOc2c(F)cc(-c3nc(Nc4ccc(CN5CC6(CN(C)C6)C5)cn4)ncc3F)cc21] |
| 57 | 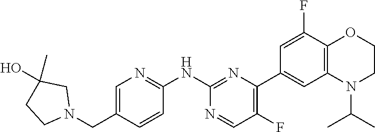 [SMILES: CC(C)N1CCOc2c(F)cc(-c3nc(Nc4ccc(CN5CCC(C)(O)C5)cn4)ncc3F)cc21] |
| 58 | 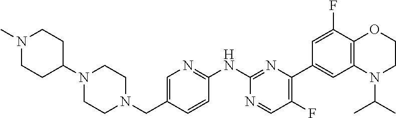 [SMILES: CC(C)N1CCOc2c(F)cc(-c3nc(Nc4ccc(CN5CCN(C6CCN(C)CC6)CC5)cn4)ncc3F)cc21] |
| 59 | 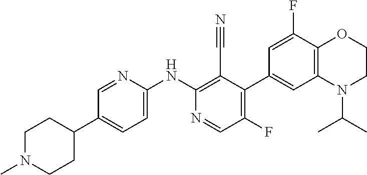 [SMILES: CC(C)N1CCOc2c(F)cc(-c3c(F)cnc(Nc4ccc(C5CCN(C)CC5)cn4)c3C#N)cc21] |
| 60 | 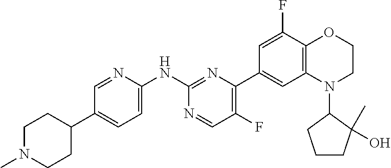 [SMILES: CN1CCC(c2ccc(Nc3ncc(F)c(-c4cc(F)c5c(c4)N(C4CCCC4(C)O)CCO5)n3)nc2)CC1] |
| 61 | 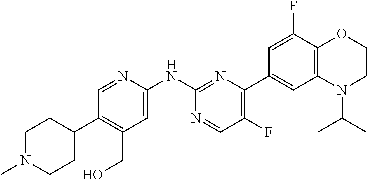 [SMILES: CC(C)N1CCOc2c(F)cc(-c3nc(Nc4cc(CO)c(C5CCN(C)CC5)cn4)ncc3F)cc21] |
| 62 | 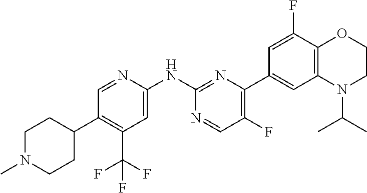 [SMILES: CC(C)N1CCOc2c(F)cc(-c3nc(Nc4cc(C(F)(F)F)c(C5CCN(C)CC5)cn4)ncc3F)cc21] |
| 63 | 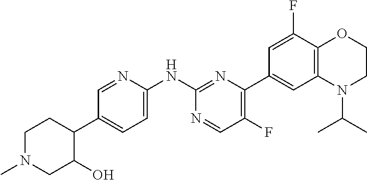 [SMILES: CC(C)N1CCOc2c(F)cc(-c3nc(Nc4ccc(C5CCN(C)CC5O)cn4)ncc3F)cc21] |
| 64 | 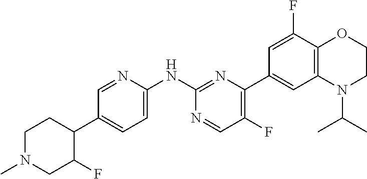 [SMILES: CC(C)N1CCOc2c(F)cc(-c3nc(Nc4ccc(C5CCN(C)CC5F)cn4)ncc3F)cc21] |
| 65 | 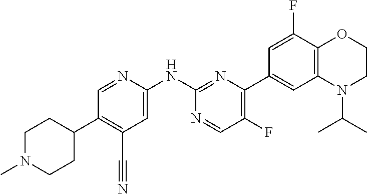 [SMILES: CC(C)N1CCOc2c(F)cc(-c3nc(Nc4cc(C#N)c(C5CCN(C)CC5)cn4)ncc3F)cc21] |
| 66 | 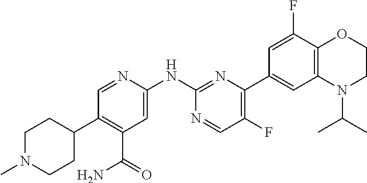 [SMILES: CC(C)N1CCOc2c(F)cc(-c3nc(Nc4cc(C(N)=O)c(C5CCN(C)CC5)cn4)ncc3F)cc21] |
| 67 | 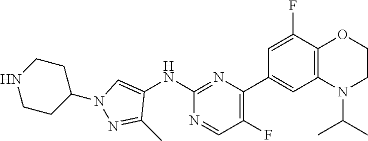 [SMILES: Cc1nn(C2CCNCC2)cc1Nc1ncc(F)c(-c2cc(F)c3c(c2)N(C(C)C)CCO3)n1] |
| 68 | 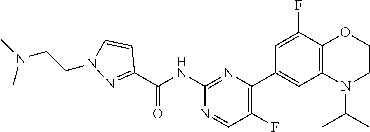 [SMILES: CC(C)N1CCOc2c(F)cc(-c3nc(NC(=O)c4ccn(CCN(C)C)n4)ncc3F)cc21] |
| 69 | 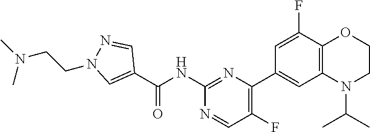 [SMILES: CC(C)N1CCOc2c(F)cc(-c3nc(NC(=O)c4cnn(CCN(C)C)c4)ncc3F)cc21] |
| 70 | 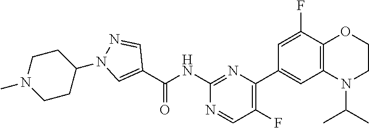 [SMILES: CC(C)N1CCOc2c(F)cc(-c3nc(NC(=O)c4cnn(C5CCN(C)CC5)c4)ncc3F)cc21] |
| 71 | 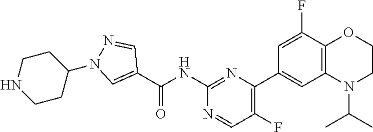 [SMILES: CC(C)N1CCOc2c(F)cc(-c3nc(NC(=O)c4cnn(C5CCNCC5)c4)ncc3F)cc21] |
| 72 | 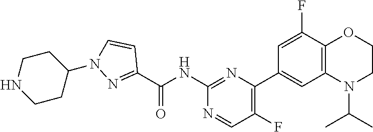 [SMILES: CC(C)N1CCOc2c(F)cc(-c3nc(NC(=O)c4ccn(C5CCNCC5)n4)ncc3F)cc21] |
| 73 | 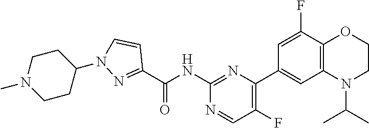 [SMILES: CC(C)N1CCOc2c(F)cc(-c3nc(NC(=O)c4ccn(C5CCN(C)CC5)n4)ncc3F)cc21] |
| 74 | 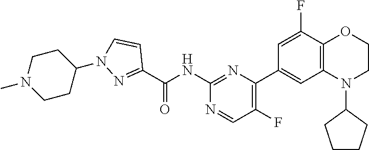 [SMILES: CN1CCC(n2ccc(C(=O)Nc3ncc(F)c(-c4cc(F)c5c(c4)N(C4CCCC4)CCO5)n3)n2)CC1] |
| 75 | 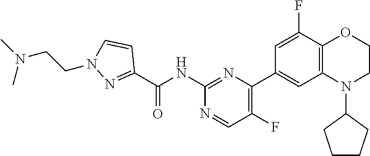 [SMILES: CN(C)CCn1ccc(C(=O)Nc2ncc(F)c(-c3cc(F)c4c(c3)N(C3CCCC3)CCO4)n2)n1] |
| 76 | 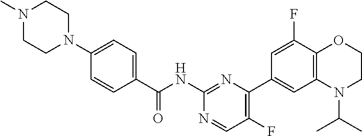 [SMILES: CC(C)N1CCOc2c(F)cc(-c3nc(NC(=O)c4ccc(N5CCN(C)CC5)cc4)ncc3F)cc21] |
| 77 | 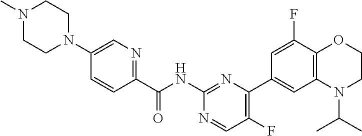 [SMILES: CC(C)N1CCOc2c(F)cc(-c3nc(NC(=O)c4ccc(N5CCN(C)CC5)cn4)ncc3F)cc21] |
| 78 | 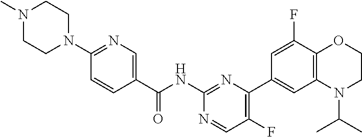 [SMILES: CC(C)N1CCOc2c(F)cc(-c3nc(NC(=O)c4ccc(N5CCN(C)CC5)nc4)ncc3F)cc21] |
| 79 | 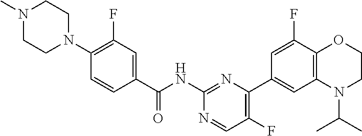 [SMILES: CC(C)N1CCOc2c(F)cc(-c3nc(NC(=O)c4ccc(N5CCN(C)CC5)c(F)c4)ncc3F)cc21] |
| 80 | 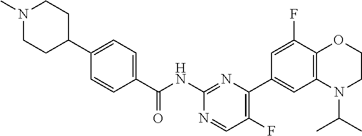 [SMILES: CC(C)N1CCOc2c(F)cc(-c3nc(NC(=O)c4ccc(C5CCN(C)CC5)cc4)ncc3F)cc21] |
| 81 | 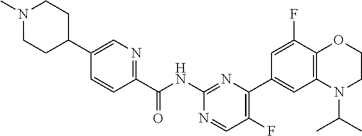 [SMILES: CC(C)N1CCOc2c(F)cc(-c3nc(NC(=O)c4ccc(C5CCN(C)CC5)cn4)ncc3F)cc21] |
| 82 | 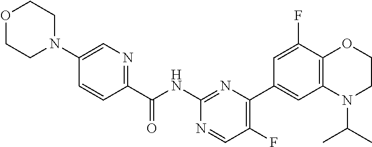 [SMILES: CC(C)N1CCOc2c(F)cc(-c3nc(NC(=O)c4ccc(N5CCOCC5)cn4)ncc3F)cc21] |
| 83 | 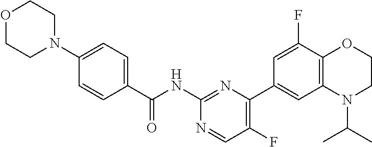 [SMILES: CC(C)N1CCOc2c(F)cc(-c3nc(NC(=O)c4ccc(N5CCOCC5)cc4)ncc3F)cc21] |
| 84 | 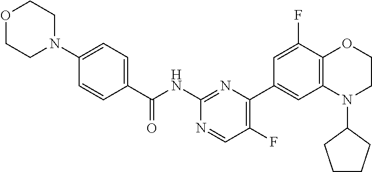 [SMILES: O=C(Nc1ncc(F)c(-c2cc(F)c3c(c2)N(C2CCCC2)CCO3)n1)c1ccc(N2CCOCC2)cc1] |
| 85 | 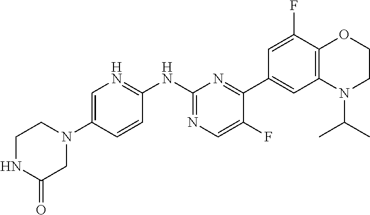 |
| 86 | 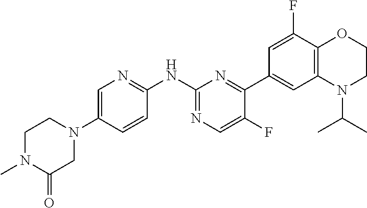 [SMILES: CC(C)N1CCOc2c(F)cc(-c3nc(Nc4ccc(N5CCN(C)C(=O)C5)cn4)ncc3F)cc21] |
| 87 | 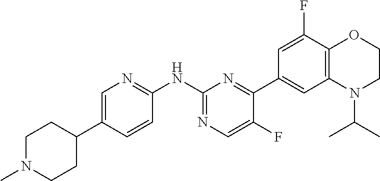 [SMILES: CC(C)N1CCOc2c(F)cc(-c3nc(Nc4ccc(C5CCN(C)CC5)cn4)ncc3F)cc21] |

In some embodiments, provided herein are compounds described in Table 1, or a tautomer thereof, or a salt of any of the foregoing, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salt % even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible stereoisomers of a compound depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol.

In some embodiments, compounds of the Formula (I) may be synthesized according to Scheme 1 to 9.

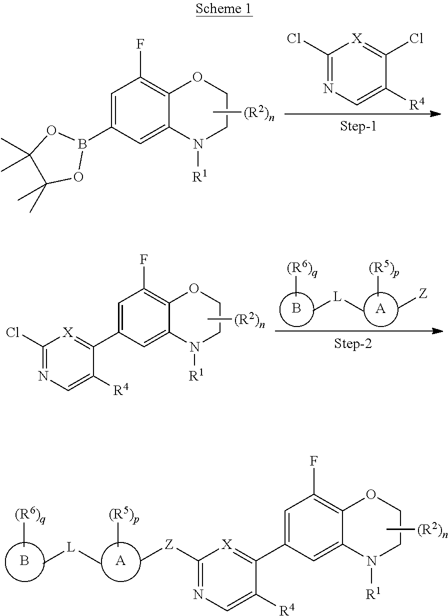

wherein A, B, L, X, Z, R¹, R², R⁴, R⁵, R⁶, n, p and q are as described for Formula (I).

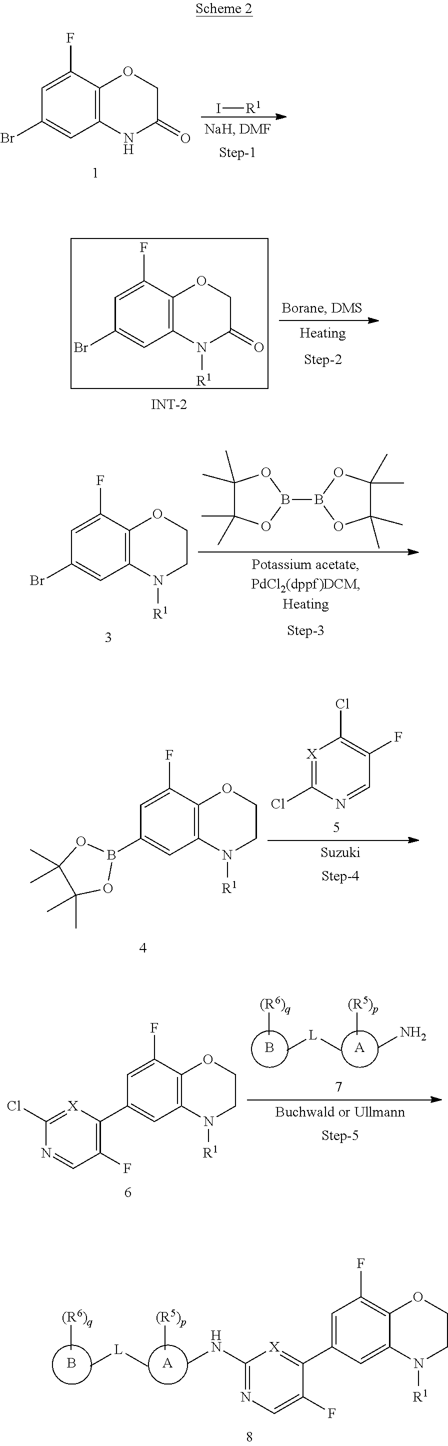

wherein A, B, L, X, R¹, R⁵, R⁶, p and q are as described for Formula (I).

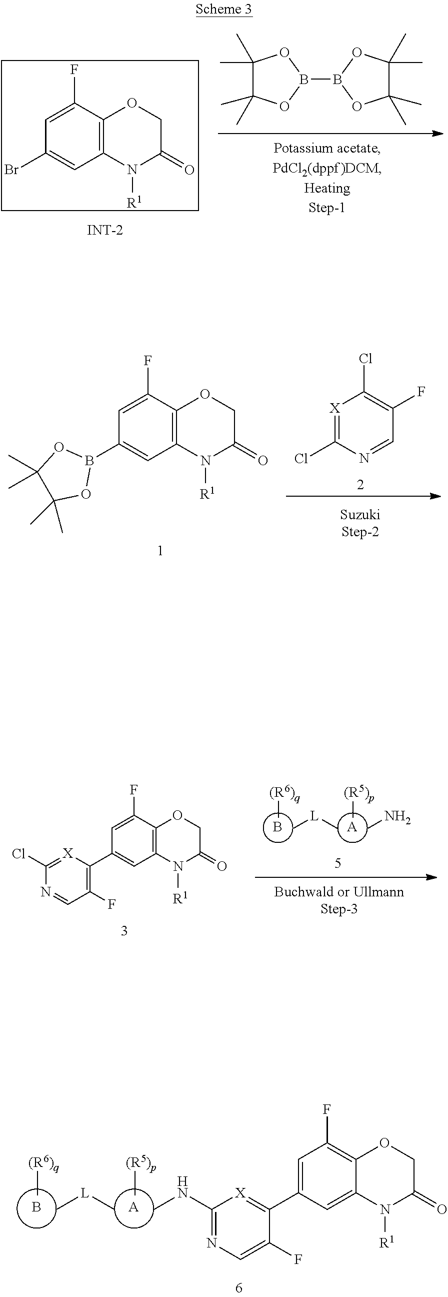

wherein A, B, L, X, R¹, R⁵, R⁶, p and q are as described for Formula (I).

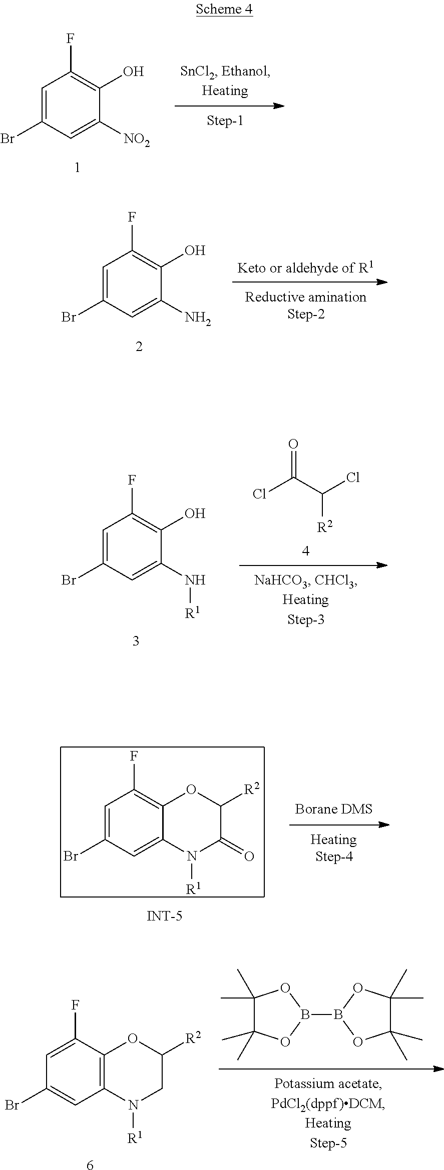

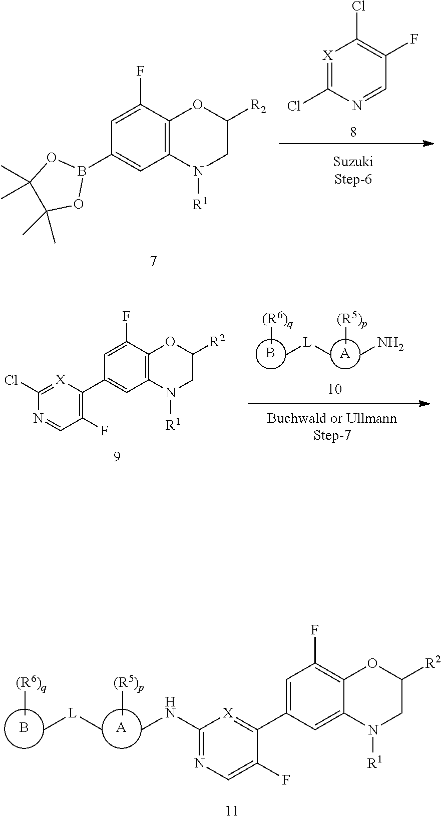

wherein A, B, L, X, R¹. R², R⁵, R⁶, p and q are as described for Formula (I).

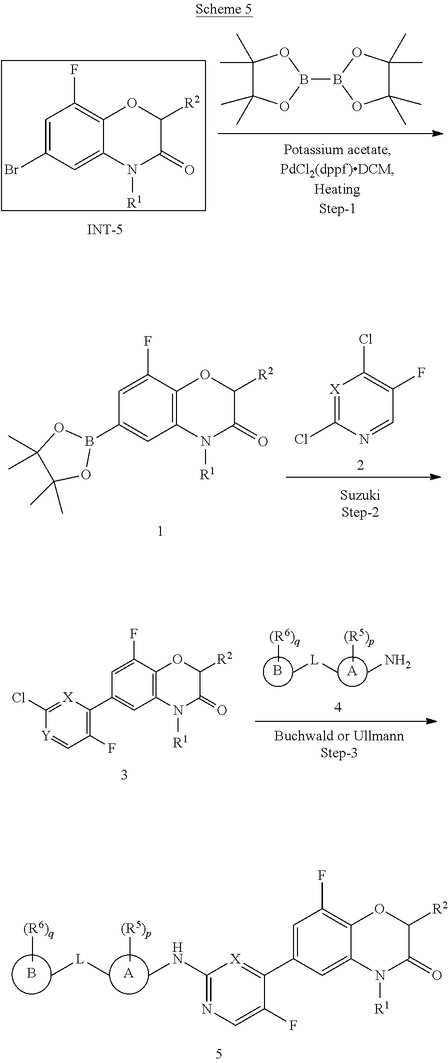

wherein A, B, L, X; R¹, R², R⁵. R⁶, p and q are as described for Formula (I).

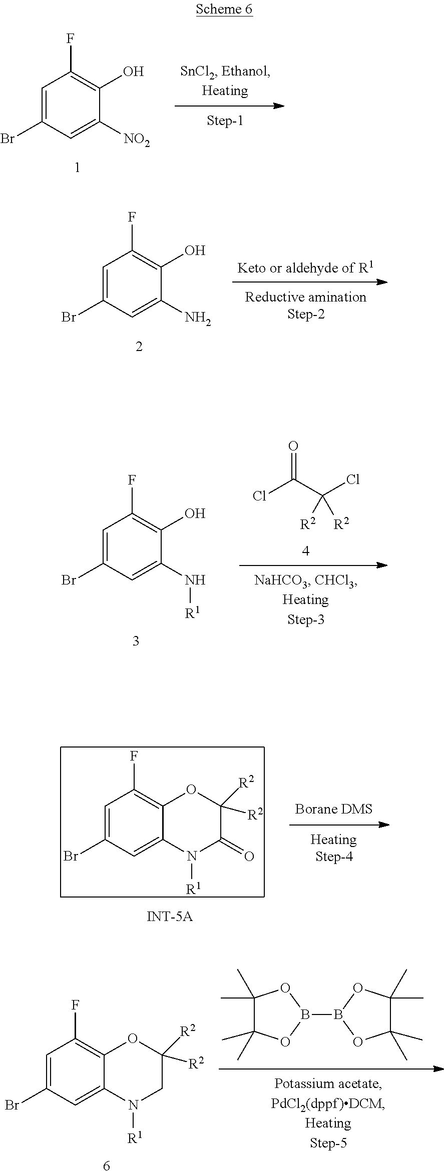

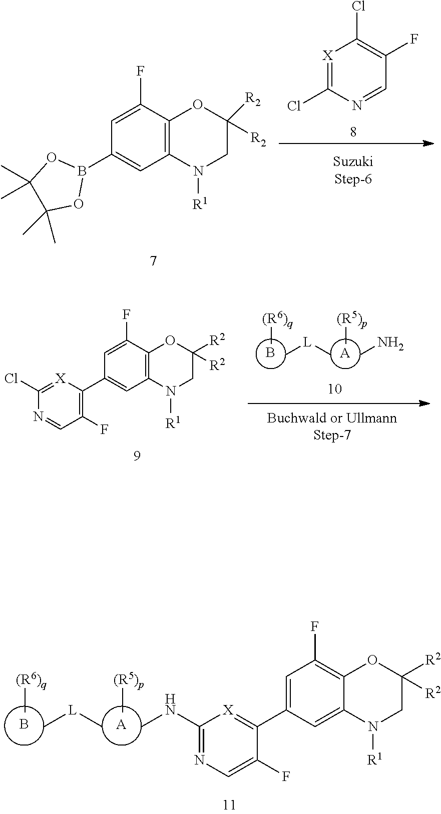

wherein A, B, L, X, R¹, R², R⁵, R⁶, p and q are as described for Formula (I).

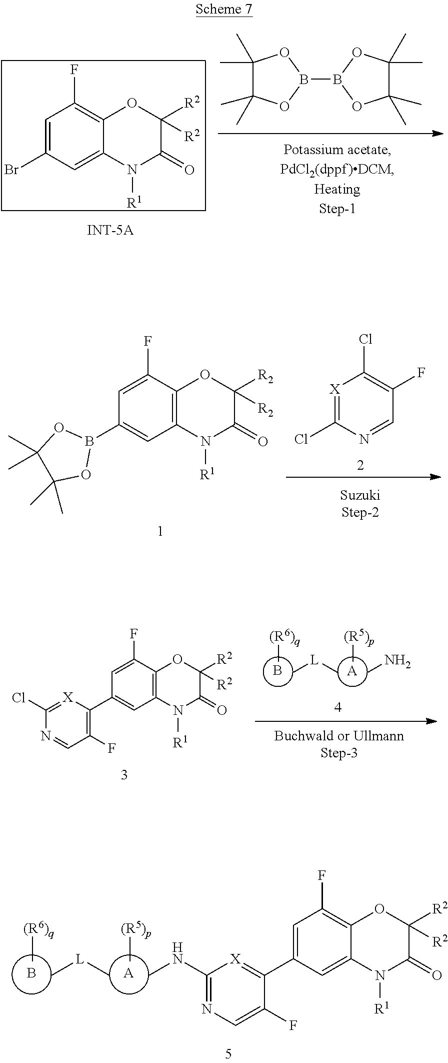

wherein A, B, L, X, R¹, R², R⁵, R⁶; p and q are as described for Formula (I). Particular examples are provided in the Example Section below.

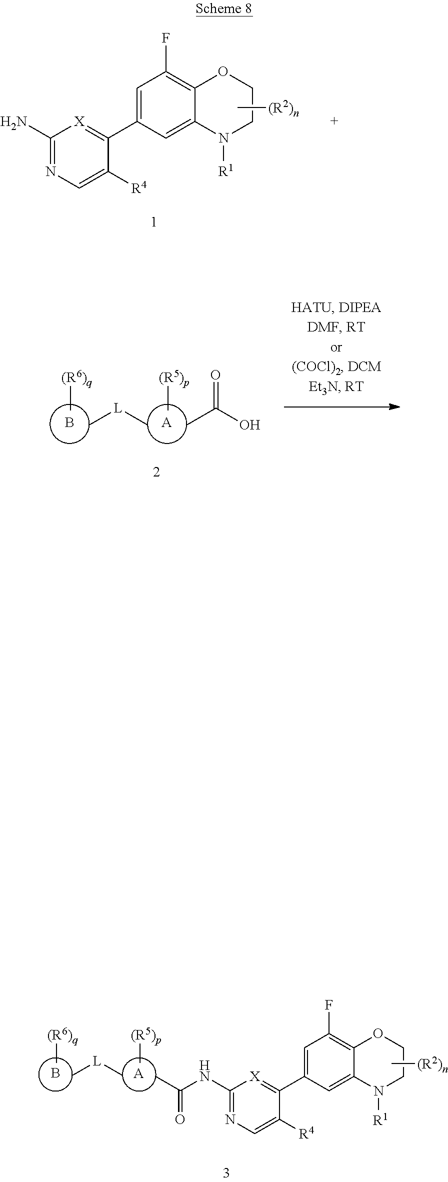

wherein A, B, L, X, R¹, R², R⁴, R⁵, R⁶; p and q are as described for Formula (I).

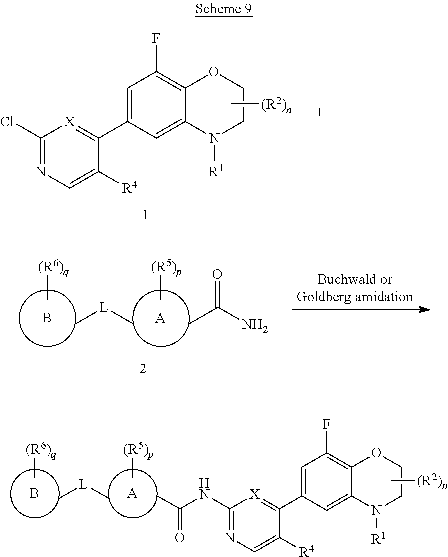

wherein A. B, L, X, R¹, R², R⁴, R⁵, R⁶; p and q are as described for Formula (I).

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences. Mack Publishing Company, Philadelphia, Pa, 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays. In some embodiments of the methods detailed herein, the methods comprise administration of a compound detailed herein, or a salt thereof, as a monotherapy.

Provided herein is a method of treating a disease in an individual comprising administering an effective amount of a compound of Formula (I), (IA), (I-B1) to (I-B20), (I-C1)-(I-C45) or any embodiment, variation or aspect thereof (collectively, a compound of Formula (I), (I-A), (I-B1) to (I-B20), (I-C1)-(I-C45)) or the present compounds or the compounds detailed or described herein) or a pharmaceutically acceptable salt thereof, to the individual. Further provided herein is a method of treating a proliferative disease in an individual, comprising administering an effective amount of the compound of Formula (I), (I-A), (I-B1) to (I-B20), (I-C1)-(I-C45), or a pharmaceutically acceptable salt thereof, to the individual. Also provided herein is a method of treating cancer in an individual comprising administering an effective amount of the compound of Formula (I), (I-A), (I-B1) to (I-B20). (I-C1)-(I-C45) or a pharmaceutically acceptable salt thereof, to the individual. In some embodiments, the compound is administered to the individual according to a dosage and/or method of administration described herein.

In some embodiments, the cancer in the individual has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of one or more of CDK1, CDK2, CDK4, CDK6 and CDK9. In some embodiments, the cancer in the individual has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of CDK4/6 and one or more of CDK1, CDK2, and CDK9.

In some embodiments, there is provided a method of treating a cancer in an individual, comprising (a) selecting the individual for treatment based on (i) the presence of phosphorylation of the retinoblastoma (Rb) protein in the cancer, or (ii) presence of mutations or amplification or overexpression of CDK4 or CDK6 in the cancer, and administering an effective amount of the compound of Formula (I), (I-A), (I-B1) to (I-B20), (I-C1)-(I-C45), or a pharmaceutically acceptable salt thereof, to the individual. In some embodiments, the cancer is assayed for the expression of phosphorylated Rb. In some embodiments, the cancer is assayed for the expression of CDK4 or CDK6. In some embodiments, the CDK4 or CDK6 gene of the cancer is sequenced to detect the one or more mutations or amplifications. In some embodiments, the CDK4 or CDK6 gene is sequenced by biopsying the cancer and sequencing the CDK4 or CDK6 gene from the biopsied cancer. In some embodiments, the CDK4 or CDK6 gene is sequenced by sequencing circulating-tumor DNA (ctDNA) from the individual.

In some embodiments, provided herein is a method of using a compound of Formula (I), (I-A), (I-B1) to (I-B20), (I-C1)-(I-C45) or any embodiment in the manufacture of a medicament for treatment of a disease. In some embodiments, provided herein is a method of using a compound of Formula (I), (I-A), (I-B1) to (I-B20), (I-C1)-(I-C45) or any embodiment in the manufacture of a medicament for treatment of cancer.

In some embodiments, a compound of Formula (I), (I-A), (I-B1) to (I-B20), (I-C1)-(I-C45) or a salt thereof is used to treat an individual having a proliferative disease, such as cancer as described herein. In some embodiments, the individual is at risk of developing a proliferative disease, such as cancer. In some of these embodiments, the individual is determined to be at risk of developing cancer based upon one or more risk factors. In some of these embodiments, the risk factor is a family history and/or gene associated with cancer.

The present compounds or salts thereof are believed to be effective for treating a variety of diseases and disorders. For example, in some embodiments, the present compositions may be used to treat a proliferative disease, such as cancer. In some embodiments the cancer is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma, cutaneous T cell lymphoma, Hodgkin's lymphoma, non-Hodgkin's lymphoma, cancer of the adrenal cortex, ACTH-producing tumors, lung cancer, including small cell carcinoma and nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In some embodiments, the cancer is defined by a molecular characteristic. In some embodiments, the cancer is an estrogen receptor-positive breast cancer. In some embodiments, the breast cancer is triple negative breast cancer. In some embodiments, the cancer is a KRAS-mutant non-small cell lung cancer. In some embodiments, the cancer is mantle cell lymphoma defined by a translocation involving CCND1 resulting in cyclin D1 overexpression.

In some embodiments, the compounds and compositions described herein cause G₁-S cell cycle arrest in a cell (such as a cancer cell). In some embodiments, the cancer cell is a cancer cell from any of the cancer types described herein. In some embodiments, arrested cells enter a state of apoptosis. In some embodiments, arrested cells enter a state of senescence. In some embodiments, provided herein is a method of causing G₁-S checkpoint arrest in a cell comprising administering an effective amount of the compound of Formula (I), (I-A), (I-B1) to (I-B20), (I-C1)-(I-C45) or a pharmaceutically acceptable salt thereof, to the cell. In some embodiments, the G₁-S cell cycle arrest occurs in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, the G₁-S cell cycle arrest occurs in up to about 99%, up to about 98%, up to about 97%, up to about 96%, up to about 95%, up to about 90%, up to about 85%, or up to about 80% of cells in the cell population.

In some embodiments, provided herein is a method of inducing senescence in a cell comprising administering an effective amount of the compound of Formula (I), (I-A), (I-B1) to (I-B20), (I-C1)-(I-C45) or a pharmaceutically acceptable salt thereof, to the cell. In some embodiments, senescence is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, senescence is induced in up to about 99%, up to about 98%, up to about 97%, up to about 96%, up to about 95%, up to about 90%, up to about 85%, or up to about 80% of cells in the cell population.

In some embodiments, provided herein is a method of inducing apoptosis in a cell comprising administering an effective amount of the compound of Formula (I), (I-A), (I-B1) to (I-B20), (I-C1)-(I-C45)) or a pharmaceutically acceptable salt thereof, to the cell. In some embodiments, apoptosis is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, apoptosis is induced in up to about 99%, up to about 98%, up to about 97%, up to about 96%, up to about 95%, up to about 90%, up to about 85%, or up to about 80% of cells in the cell population.

In some embodiments, provided herein is a method of inhibiting CDK4 or CDK6 in a cell comprising administering an effective amount of the compound of Formula (I), (I-A), (I-B1) to (I-B20), (I-C1)-(I-C45) or a pharmaceutically acceptable salt thereof, to the cell. In some embodiments, CDK4 or CDK6 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, CDK4 or CDK6 is inhibited up to about 99%, up to about 98%, up to about 97%, up to about 96%, up to about 95%, up to about 90%, up to about 85%, up to about 80%, up to about 70%, or up to about 60%. In some embodiments, the activity of CDK4 or CDK6 is measured according to a kinase assay.

In some embodiments, provided herein is a method of inhibiting one or more of CDK1, CDK2, CDK4, CDK6, and CDK9 in a cell comprising administering an effective amount of the compound of Formula (I), (I-A), (I-B1) to (I-B20), (I-C1)-(I-C45) or a pharmaceutically acceptable salt thereof, to the cell. In some embodiments, one or more of CDK1, CDK2, CDK4, CDK6, and CDK9 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, one or more of CDK1, CDK2. CDK4, CDK6, and CDK9 is inhibited up to about 99%, up to about 98%, up to about 97%, up to about 96%, up to about 95%, up to about 90%, up to about 85%, up to about 80%, up to about 70%, or up to about 60%. In some embodiments, the activity of one or more of CDK1. CDK2, CDK4, CDK6, and CDK9 is measured according to a kinase assay.

In some embodiments, provided herein is a method of inhibiting CDK4 or CDK6 comprising contacting CDK4 or CDK6 with an effective amount of the compound of Formula (I), (I-A), (I-B1) to (I-B20), (I-C1)-(I-C45) or a pharmaceutically acceptable salt thereof. In some embodiments, the compound of Formula (I), (I-A), (I-B1) to (I-B20), (I-C1)-(I-C45) or a pharmaceutically acceptable salt thereof binds to CDK4 or CDK6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (I), (I-A), (I-B1) to (I-B20), (I-C1)-(I-C45) or a pharmaceutically acceptable salt thereof binds to CDK4 or CDK6 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay.

In some embodiments, provided herein is a method of inhibiting one or more of CDK1, CDK2, CDK4, CDK6, and CDK9 comprising contacting one or more of CDK1, CDK2, CDK4, CDK6, and CDK9 with an effective amount of the compound of Formula (I). (I-A), (I-B1) to (I-B20), (I-C1)-(I-C45) or a pharmaceutically acceptable salt thereof. In some embodiments, the compound of Formula (I), (IA), (I-B1) to (I-B20), (I-C1)-(I-C45) or a pharmaceutically acceptable salt thereof binds to one or more of CDK1, CDK2, CDK4, CDK6, and CDK9 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (I), (I-A), (I-B1) to (I-B20), (I-C1)-(I-C45) or a pharmaceutically acceptable salt thereof binds to one or more of CDK1, CDK2, CDK4, CDK6, and CDK9 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay.

In some embodiments, provided herein is a method of modulating CDK4/6 in an individual, comprising administering to the individual a compound of Formula (I), (I-A), (I-B1) to (I-B20), (I-C1)-(I-C45) or a pharmaceutically acceptable salt thereof, or a salt thereof. In some embodiments, provided herein is a method of modulating CDK4 and CDK 6 in an individual, comprising administering to the individual a compound of Formula (I), (I-A), (I-B1) to (I-B20), (I-C1)-(I-C45) or a pharmaceutically acceptable salt thereof, or a salt thereof. In some embodiments, provided herein is a method of modulating CDK4/6 and one or more of CDK1, CDK2, and CDK9 in an individual, comprising administering to the individual a compound detailed herein, or a salt thereof. In some embodiments, provided herein is a method of modulating CDK4 and CDK 6 and one or more of CDK1. CDK2, and CDK9 in an individual, comprising administering to the individual a compound detailed herein, or a salt thereof. In some embodiments, the compound of Formula (I), (I-A), (I-B1) to (I-B20), (I-C1)-(I-C45) or a pharmaceutically acceptable salt thereof binds to one or more of CDK4/6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (I), (I-A), (I-B1) to (I-B20), (I-C1)-(I-C45) or a pharmaceutically acceptable salt thereof binds to one or more of CDK4 and CDK6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, the compound of Formula (I), (I-A), (I-B1) to (I-B20), (I-C1)-(I-C45) or a pharmaceutically acceptable salt thereof binds to one or more of CDK1, CDK2, CDK4. CDK6, and CDK9 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay.

In one embodiment, the compound or a salt thereof may enhance the antitumour immunity by increasing the functional capacity of tumour cells to present antigen or by reducing the immunosuppressive T_{Reg} population by suppressing their proliferation.

In some embodiments, provided herein is a method of inhibiting the proliferation of a cell, comprising contacting the cell with an effective amount of the compound of Formula (I), (I-A), (I-B1) to (I-B20), (I-C1)-(I-C45) or a pharmaceutically acceptable salt thereof. In some embodiments, the compound of Formula (I), (IA), (I-B20) to (I-B12), (I-C1)-(I-C45) or a pharmaceutically acceptable salt thereof is effective in inhibiting the proliferation of the cell with an EC₅₀ of less than 5 μM, less than 2 μM, less than 1 μM, less than 900 nM, less than 800 nM, less than 700 nM, less than 600 nM, less than 500 nM, less than 400 nM, less than 300 nM, less than 200 nM, less than 100 nM, or less than 50 nM. In some embodiments, the compound of Formula (I), (I-A), (I-B1) to (I-B20), (I-C1)-(I-C45) or a pharmaceutically acceptable salt is effective in inhibiting the proliferation of the cell with an EC₅₀ between 10 nM and 20 nM, between 20 nM and 50 nM, between 50 nM and 100 nM, between 100 nM and 500 nM, between 500 nM and 1 μM, between 1 μM and 2 μM, or between 2 μM and 5 μM. In some embodiments, the EC₅₀ is measured according to a cell proliferation assay.

### Combination Therapy

As provided herein, the presently disclosed compounds or a salt thereof may affect the immune system. Accordingly, the present compounds or a salt thereof may be used in combination with other anti-cancer agents or immunotherapies. In some embodiments, provided herein is a method of treating a disease in an individual comprising administering an effective amount of a compound of Formula (I), (I-A), (I-B1) to (I-B20), (I-C1)-(I-C45), or any embodiment, variation or aspect thereof (collectively, a compound of Formula (I), (I-A). (I-B1) to (I-B20), (I-C1)-(I-C45) or the present compounds or the compounds detailed or described herein) or a pharmaceutically acceptable salt thereof, and an additional therapeutic agent to the individual. In some embodiments, the second therapeutic agent is a cancer immunotherapy agent or an endocrine therapy agent or a chemotherapeutic agent. In some embodiments, the disease is a proliferative disease such as cancer.

In some embodiments, the additional therapeutic agent is a cancer immunotherapy agent. In some embodiments, the additional therapeutic agent is an immunostimulatory agent. In some embodiments, the additional therapeutic agent targets a checkpoint protein (for example an immune checkpoint inhibitor). In some embodiments, the additional therapeutic agent is effective to stimulate, enhance or improve an immune response against a tumor.

In another aspect provided herein is a combination therapy for the treatment of a disease, such as cancer. In some embodiments, there is provided a method of treating a disease in an individual comprising administering an effective amount of Formula (I), (IA), (I-B1) to (I-B20), (I-C1)-(I-C45), or any embodiment, variation or aspect thereof (collectively, a compound of Formula (I), (IA-), (I-B1) to (I-B20), (I-C1)-(I-C45), or the present compounds or the compounds detailed or described herein) or a pharmaceutically acceptable salt thereof, in combination with a radiation therapy.

In some embodiments, there is provided a method of treating a disease in an individual comprising (a) administering an effective amount of Formula (I), (I-A), (I-B1) to (I-B20), (I-C1)-(I-C45) or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of an endocrine therapy agent. In some embodiments, the endocrine therapy is antiestrogen therapy. In some embodiments, the endocrine therapy is a selective estrogen receptor degrader (SERD, such as fulvestrant). In some embodiments, the endocrine therapy is a selective estrogen receptor modulator (SERM, such as tamoxifen). In some embodiments, the endocrine therapy is an aromatase inhibitor (such as letrozole). In some embodiments, the combination of a CDK4/6 inhibitor and endocrine therapy causes enhancement of G1-S cell-cycle arrest. In some embodiments, the combination of a CDK4/6 inhibitor and endocrine therapy causes enhanced entry into a senescent state. In some embodiments, Formula (I), (I-A), (I-B1) to (I-B20), (I-C1)-(I-C45) or a pharmaceutically acceptable salt thereof is administered prior to, after, or simultaneously co-administered with the endocrine therapy agent. In some embodiments. Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the endocrine therapy agent.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively. Formula (I), (IA-), (I-B1) to (I-B20), (I-C1)-(I-C45)) or a pharmaceutically acceptable salt thereof, and (b) administering an effective amount of a second chemotherapeutic agent. In some embodiments, the chemotherapeutic agent is another kinase inhibitor. In some embodiments, Formula (I), (IA), (I-B1) to (I-B20), (I-C1)-(I-C45) or a pharmaceutically acceptable salt thereof is administered prior to, after, or simultaneously co-administered with the second chemotherapeutic agent. In some embodiments. Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the second chemotherapeutic agent.

Examples of chemotherapeutic agents that can be used in combination with Formula (I), (I-A), (I-B1) to (I-B20), (I-C1)-(I-C45) or a pharmaceutically acceptable salt thereof include DNA-targeted agents, a DNA alkylating agent (such as cyclophosphamide, mechlorethamine, chlorambucil, melphalan, dacarbazine, or nitrosoureas), a topoisomerase inhibitor (such as a Topoisomerase I inhibitor (e.g., irinotecan or topotecan) or a Topoisomerase II inhibitor (e.g., etoposide or teniposide)), an anthracycline (such as daunorubicin, doxorubicin, epirubicin, idarubicin, mitoxantrone, or valrubicin), a histone deacetylase inhibitor (such as vorinostat or romidepsin), a bromodomain inhibitor, other epigenetic inhibitors, a taxane (such as paclitaxel or docetaxel), a kinase inhibitor (such as bortezomib, erlotinib, gefitinib, imatinib, vemurafenib, vismodegib, ibrutinib), an anti-angiogenic inhibitor, a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine), or a platinum-based chemotherapeutic agent (such as cisplatin, carboplatin, or oxaliplatin), pemetrexed, or a combination thereof. In some embodiments, there is provide a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a kinase inhibitor (such as bortezomib, erlotinib, gefitinib, imatinib, vemurafenib, vismodegib, or ibrutinib). In some embodiments, Formula (I), (I-A), (I-B1) to (I-B20), (I-C1)-(I-C45) or a pharmaceutically acceptable salt thereof is administered prior to, after, or simultaneously co-administered with the kinase inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the kinase inhibitor.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a DNA damaging agent. In some embodiments, Formula (I), (I-A), (I-B1) to (I-B20), (I-C1)-(I-C45) or a pharmaceutically acceptable salt thereof is administered prior to, after, or simultaneously co-administered with the DNA damaging agent. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the DNA damaging agent.

In some embodiments, there is provided a method of treating a disease in an individual comprising (a) administering an effective amount of Formula (I), (I-A), (I-B1) to (I-B20), (I-C1)-(I-C45)), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a DNA alkylating agent (such as cyclophosphamide, mechlorethamine, chlorambucil, melphalan, dacarbazine, or nitrosoureas). In some embodiments, Formula (I), (I-A), (I-B1) to (I-B20), (I-C1)-(I-C45) or a pharmaceutically acceptable salt thereof is administered prior to, after, or simultaneously co-administered with the DNA alkylating agent. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the DNA alkylating agent.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a topoisomerase inhibitor (such as a Topoisomerase 1 inhibitor (e.g., irinotecan or topotecan) or a Topoisomerase 11 inhibitor (e.g., etoposide or teniposide)). In some embodiments, Formula (I), (I-A), (I-B1) to (I-B20), (I-C1)-(I-C45) or a pharmaceutically acceptable salt thereof is administered prior to, after, or simultaneously co-administered with the topoisomerase inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the topoisomerase inhibitor.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of an anthracycline (such as daunorubicin, doxorubicin, epirubicin, idarubicin, mitoxantrone, or valrubicin). In some embodiments, Formula (I), (I-A), (I-B1) to (I-B20), (I-C1)-(I-C45) or a pharmaceutically acceptable salt thereof is administered prior to, after, or simultaneously co-administered with the anthracycline. In some embodiments, Formula (I), (IA), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the anthracycline.

In some embodiments, there is provide a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a histone deacetylase inhibitor (such as vorinostat or romidepsin). In some embodiments, Formula I or a pharmaceutically acceptable salt thereof is administered prior to, after, or simultaneously co-administered with the histone deacetylase inhibitor. In some embodiments. Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the histone deacetylase inhibitor.

In some embodiments, there is provide a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a taxane (such as paclitaxel or docetaxel). In some embodiments. Formula (I), (I-A), (I-B1) to (I-B20), (I-C1)-(I-C45) or a pharmaceutically acceptable salt thereof is administered prior to, after, or simultaneously co-administered with the taxane. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the taxane.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively. Formula (I), (I-A), (I-B1) to (I-B20), (I-C1)-(I-C45)) or a pharmaceutically acceptable salt thereof, and (b) administering an effective amount of a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine). In some embodiments, Formula (I), (I-A), (I-B1) to (I-B20), (I-C1)-(I-C45) or a pharmaceutically acceptable salt thereof is administered prior to, after, or simultaneously co-administered with the nucleotide analog or precursor analog. In some embodiments, Formula (I), (IA), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the nucleotide analog or precursor analog.

In some embodiments, there is provided a method of treating a disease in an individual comprising (a) administering an effective amount of Formula (I), (IA), (I-B1) to (I-B20), (I-C1)-(I-C45), or any embodiment, variation or aspect thereof (collectively. Formula (I), (I-A), (I-B1) to (I-B20), (I-C1)-(I-C45)) or a pharmaceutically acceptable salt thereof, and (b) administering an effective amount of a platinum-based chemotherapeutic agent (such as cisplatin, carboplatin, or oxaliplatin). In some embodiments, Formula (I), (I-A), (I-B1) to (I-B20), (I-C1)-(I-C45) or a pharmaceutically acceptable salt thereof is administered prior to, after, or simultaneously co-administered with the platinum-based chemotherapeutic agent. In some embodiments. Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the platinum-based chemotherapeutic agent.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively. Formula (I), (I-A), (I-B1) to (I-B20), (I-C1)-(I-C45)) or a pharmaceutically acceptable salt thereof, and (b) administering an effective amount of pemetrexed. In some embodiments. Formula (I), (I-A), (I-B1) to (I-B20), (I-C1)-(I-C45) or a pharmaceutically acceptable salt thereof is administered prior to, after, or simultaneously co-administered with the pemetrexed. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the pemetrexed.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a Bruton's tyrosine kinase (BTK) inhibitor. In some embodiments. Formula (I), (I-A), (I-B1) to (I-B20), (I-C1)-(I-C45) or a pharmaceutically acceptable salt thereof is administered prior to, after, or simultaneously co-administered with the BTK inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the BTK inhibitor.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a PI3K or Akt inhibitor. In some embodiments, Formula (I), (I-A), (I-B1) to (I-B20), (I-C1)-(I-C45) or a pharmaceutically acceptable salt thereof is administered prior to, after, or simultaneously co-administered with the PI3K or Akt inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the PI3K or Akt inhibitor.

In some embodiments, there is provided a method of treating a disease in an individual comprising (a) administering an effective amount of Formula (I), (I-A), (I-B1) to (I-B20), (I-C1)-(I-C45)), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a DNA damage repair (DDR) pathway inhibitor. In some embodiments, Formula (I), (I-A), (I-B1) to (I-B20), (I-C1)-(I-C45) or a pharmaceutically acceptable salt thereof is administered prior to, after, or simultaneously co-administered with the DDR pathway inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the DDR pathway inhibitor. Examples of inhibitors of the DDR pathway include poly(ADP-ribose) polymerase (PARP) inhibitors (such as olaparib, rucaparib, niraparib, or talazoparib), ataxia telangiectasia mutated (ATM) protein inhibitors, ataxia telangiectasia and Rad3-related (ATR) protein inhibitors, checkpoint kinase 1 (Chk1) inhibitors, or combinations thereof.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a PARP inhibitor (such as olaparib, rucaparib, niraparib, or talazoparib). In some embodiments. Formula (I), (IA), (I-B1) to (I-B20), (I-C1)-(I-C45) or a pharmaceutically acceptable salt thereof is administered prior to, after, or simultaneously co-administered with the PARP inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the PARP inhibitor.

In some embodiments, there is provided a method of treating a disease in an individual comprising (a) administering an effective amount of Formula (I), (I-A), (I-B1) to (I-B20), (I-C1)-(I-C45)), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of an ATM protein inhibitor. In some embodiments, Formula (I), (I-A), (I-B1) to (I-B20), (I-C1)-(I-C45) or a pharmaceutically acceptable salt thereof is administered prior to, after, or simultaneously co-administered with the ATM protein inhibitor. In some embodiments, Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the ATM protein inhibitor.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of an ATR protein inhibitor. In some embodiments, Formula (I), (I-A), (I-B1) to (I-B20), (I-C1)-(I-C45) or a pharmaceutically acceptable salt thereof is administered prior to, after, or simultaneously co-administered with the ATR protein inhibitor. In some embodiments, Formula (I), (IA), (I-B1) to (I-B20), (1-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the ATR protein inhibitor.

In some embodiments, there is provided a method of treating a disease in an individual comprising (a) administering an effective amount of Formula (I), (I-A), (I-B1) to (I-B20), (I-C1)-(I-C45), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of an Chk1 inhibitor. In some embodiments, Formula (I), (I-A), (I-B1) to (I-B20), (I-C1)-(I-C45) or a pharmaceutically acceptable salt thereof is administered prior to, after, or simultaneously co-administered with the Chk1 inhibitor. In some embodiments. Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the Chk1 inhibitor.

In some embodiments, there is provided a method of treating a disease in an individual comprising (a) administering an effective amount of Formula (I), (I-A), (I-B1) to (I-B20), (I-C1)-(I-C45)), or any embodiment, variation or aspect thereof (collectively, Formula (I), (I-A), (I-B1) to (I-B20), (I-C1)-(I-C45)) or a pharmaceutically acceptable salt thereof, and (b) administering an effective amount of a further CDK4/6 inhibitor. In some embodiments, Formula (I), (I-A), (I-B1) to (I-B20), (I-C1)-(I-C45) or a pharmaceutically acceptable salt thereof is administered prior to, after, or simultaneously co-administered with the further CDK4/6 inhibitor. In some embodiments. Formula (I), (I-A), (I-B1) to (I-B20), (I-C1)-(I-C45) or a pharmaceutically acceptable salt thereof is administered 1 or more hours (such as 2 or more hours, 4 or more hours, 8 or more hours, 12 or more hours, 24 or more hours, or 48 or more hours) prior to or after the further CDK4/6 inhibitor.

In another aspect, provided herein is a combination therapy in which a compound of Formula (I), (I-A), (I-B1) to (I-B20), (I-C1)-(I-C45), or a salt thereof is coadministered (which may be separately or simultaneously) with one or more additional agents that are effective in stimulating immune responses to thereby further enhance, stimulate or upregulate immune responses in a subject. For example, provided is a method for stimulating an immune response in a subject comprising administering to the subject a compound of Formula (I), (I-A), (I-B1) to (I-B20), (I-C1)-(I-C45), or a salt thereof and one or more immunostimulatory antibodies, such as an anti-PD-1 antibody, an anti-PD-L1 antibody and/or an anti-CTLA-4 antibody, such that an immune response is stimulated in the subject, for example to inhibit tumor growth. In one embodiment, the subject is administered a compound of formula Formula (I), (I-A), (I-B1) to (I-B20), (I-C1)-(I-C45) or a salt thereof and an anti-PD-1 antibody. In another embodiment, the subject is administered a compound of Formula (I), (I-A), (I-B1) to (I-B20), (I-C1)-(I-C45), or a salt thereof and an anti-PD-L1 antibody. In yet another embodiment, the subject is administered a compound of Formula (I), (I-A), (I-B1) to (I-B20), (I-C1)-(I-C45) or a salt thereof and an anti-CTLA-4 antibody. In another embodiment, the immunostimulatory antibody (e.g., anti-PD-1, anti-PD-L1 and/or anti-CTLA-4 antibody) is a human antibody. Alternatively, the immunostimulatory antibody can be, for example, a chimeric or humanized antibody (e.g., prepared from a mouse anti-PD-1, anti-PD-L1 and/or anti-CTLA-4 antibody).

In one embodiment, the present disclosure provides a method for treating a proliferative disease (e.g., cancer), comprising administering a compound of Formula (I), (I-A), (I-B1) to (I-B20), (I-C1)-(I-C45), or a salt thereof and an anti-PD-1 antibody to a subject. In further embodiments, a compound of Formula (I), (I-A), (I-B1) to (I-B20), (I-C1)-(I-C45) or a salt thereof is administered at a subtherapeutic dose, the anti-PD-1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a compound of Formula (I), (I-A), (I-B1) to (I-B20), (I-C1)-(I-C45), or a salt thereof and a subtherapeutic dose of anti-PD-1 antibody to a subject. In certain embodiments, the subject is human. In certain embodiments, the anti-PD-1 antibody is a human sequence monoclonal antibody.

In one embodiment, the present invention provides a method for treating a hyperproliferative disease (e.g., cancer), comprising administering a compound of Formula (I), (I-A), (I-B1) to (I-B20), (I-C1)-(I-C45), or a salt thereof and an anti-PD-L1 antibody to a subject. In further embodiments, a compound of Formula (I), (I-A), (I-B1) to (I-B20), (I-C1)-(I-C45) or a salt thereof is administered at a subtherapeutic dose, the anti-PD-L1 antibody is administered at a subtherapeutic dose, or both are administered at a subtherapeutic dose. In another embodiment, the present invention provides a method for altering an adverse event associated with treatment of a hyperproliferative disease with an immunostimulatory agent, comprising administering a compound of Formula (I), (I-A), (I-B1) to (I-B20), (I-C1)-(I-C45), or a salt thereof and a subtherapeutic dose of anti-PD-L1 antibody to a subject. In certain embodiments, the subject is human. In certain embodiments, the anti-PD-L1 antibody is a human sequence monoclonal antibody.

In certain embodiments, the combination of therapeutic agents discussed herein can be administered concurrently as a single composition in a pharmaceutically acceptable carrier, or concurrently as separate compositions each in a pharmaceutically acceptable carrier. In another embodiment, the combination of therapeutic agents can be administered sequentially. For example, an anti-CTLA-4 antibody and a compound of Formula (I), (I-A), (I-B1) to (I-B20), (I-C1)-(I-C45), or a salt thereof can be administered sequentially, such as anti-CTLA-4 antibody being administered first and a compound of Formula (I), (I-A), (I-B1) to (I-B20), (I-C1)-(I-C45), or a salt thereof second, or a compound of Formula (I), (I-A), (I-B1) to (I-B20), (I-C1)-(I-C45), or a salt thereof being administered first and anti-CTLA-4 antibody second. Additionally or alternatively, an anti-PD-1 antibody and a compound of Formula (I), (I-A), (I-B1) to (I-B20), (I-C1)-(I-C45), or a salt thereof can be administered sequentially, such as anti-PD-1 antibody being administered first and a compound of Formula (I), (I-A), (I-B1) to (I-B20), (I-C1)-(I-C45), or a salt thereof second, or a compound of Formula (I), (I-A), (I-B1) to (I-B20), (I-C1)-(I-C45), or a salt thereof being administered first and anti-PD-1 antibody second. Additionally or alternatively, an anti-PD-L1 antibody and a compound of Formula (I), (I-A), (I-B1) to (I-B20), (I-C1)-(I-C45), or a salt thereof can be administered sequentially, such as anti-PD-L1 antibody being administered first and a compound of Formula (I), (I-A), (I-B1) to (I-B20), (I-C1)-(I-C45), or a salt thereof second, or a compound of Formula (I), (IA), (I-B1) to (I-B20), (I-C1)-(I-C45), or a salt thereof being administered first and anti-PD-L1 antibody second.

Furthermore, if more than one dose of the combination therapy is administered sequentially, the order of the sequential administration can be reversed or kept in the same order at each time point of administration, sequential administrations can be combined with concurrent administrations, or any combination thereof.

Optionally, the combination of a compound of Formula (I), (I-A), (I-B1) to (I-B20), (I-C1)-(I-C45), or a salt thereof can be further combined with an immunogenic agent, such as cancerous cells, purified tumor antigens (including recombinant proteins, peptides, and carbohydrate molecules), cells, and cells transfected with genes encoding immune stimulating cytokines.

A compound of Formula (I), (I-A), (I-B1) to (I-B20), (I-C1)-(I-C45), or a salt thereof can also be further combined with standard cancer treatments. For example, a compound of Formula (I), (I-A), (I-B1) to (I-B20), (I-C1)-(I-C45), or a salt thereof can be effectively combined with chemotherapeutic regimens. In these instances, it is possible to reduce the dose of other chemotherapeutic reagent administered with the combination of the instant disclosure. Other combination therapies with a compound of Formula (I), (I-A), (I-B1) to (I-B20), (I-C1)-(I-C45)), or a salt thereof include radiation, surgery, or hormone deprivation. Angiogenesis inhibitors can also be combined with a compound of Formula (I), (I-A), (I-B1) to (I-B20), (I-C1)-(I-C45), or a salt thereof. Inhibition of angiogenesis leads to tumor cell death, which can be a source of tumor antigen fed into host antigen presentation pathways.

In another example, a compound of Formula (I), (I-A), (I-B1) to (I-B20), (I-C1)-(I-C45)), or a salt thereof can be used in conjunction with anti-neoplastic antibodies. By way of example and not wishing to be bound by theory, treatment with an anti-cancer antibody or an anti-cancer antibody conjugated to a toxin can lead to cancer cell death (e.g., tumor cells) which would potentiate an immune response mediated by CTLA-4, PD-1, PD-L1 or a compound of Formula (I), (I-A), (I-B1) to (I-B20), (I-C1)-(I-C45), or a salt thereof. In an exemplary embodiment, a treatment of a hyperproliferative disease (e.g., a cancer tumor) can include an anti-cancer antibody in combination with a compound of Formula (I), (I-A), (I-B1) to (I-B20), (I-C1)-(I-C45) or a salt thereof and anti-CTLA-4 and/or anti-PD-1 and/or anti-PD-L1 antibodies, concurrently or sequentially or any combination thereof, which can potentiate anti-tumor immune responses by the host. Other antibodies that can be used to activate host immune responsiveness can be further used in combination with a compound of Formula (I), (I-A), (I-B1) to (I-B20), (I-C1)-(I-C45) or a salt thereof.

In some embodiments, a compound of Formula (I), (I-A), (I-B1) to (I-B20), (I-C1)-(I-C45), or a salt thereof can be combined with an anti-CD73 therapy, such as an anti-CD73 antibody.

In yet further embodiments, the compound of Formula (I), (I-A), (I-B1) to (I-B20), (I-C1)-(I-C45), or a salt thereof is administered in combination with another CDK4 or CDK6 inhibitor or other CDK inhibitor.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily. e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

Also provided are articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

### Synthetic Examples

### Example-1: Synthesis of N-(3-(1,4-diazepan-1-yl)phenyl)-S-fluoro-4-(8-fluoro-4-isopropyl-3,4-dihydro-2H-benzo[b][1,4]oxazin-6-yl)pyrimidin-2-amine. (Compound No. 1)

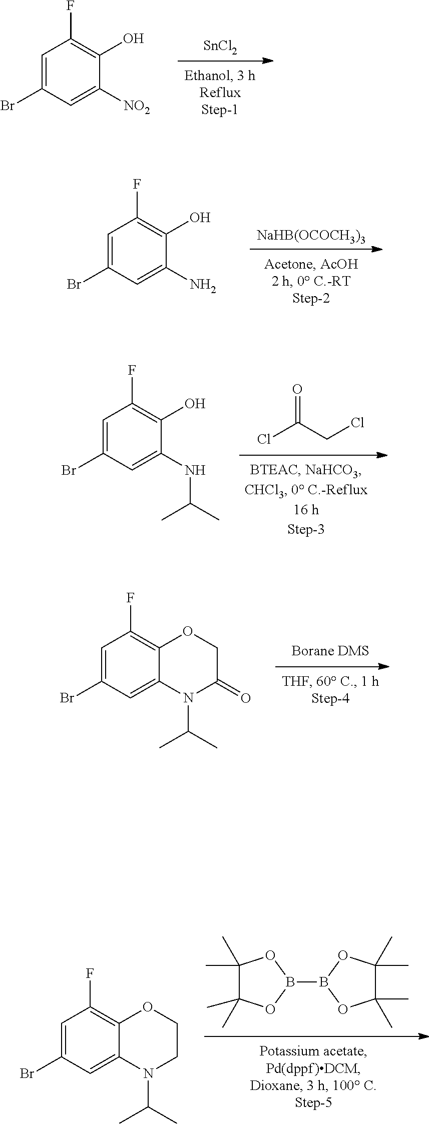

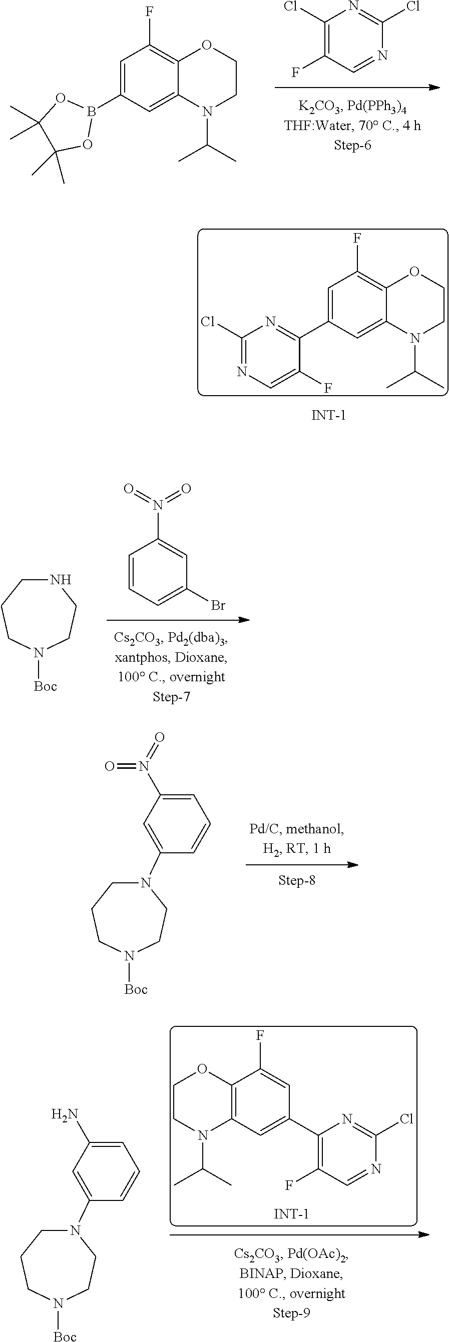

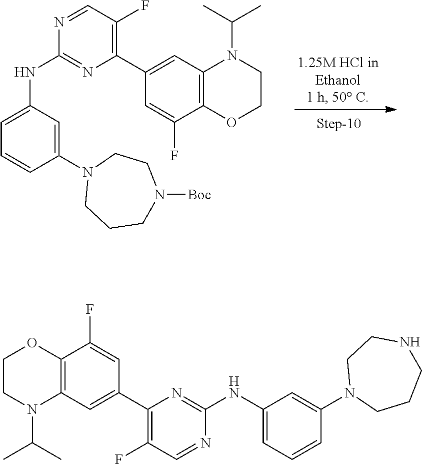 [SMILES: CC(C)N1CCOc2c(F)cc(-c3nc(Nc4cccc(N5CCCN(C(=O)OC(C)(C)C)CC5)c4)ncc3F)cc21 ; CC(C)N1CCOc2c(F)cc(-c3nc(Nc4cccc(N5CCCNCC5)c4)ncc3F)cc21]

Step-1 Synthesis of 2-amino-4-bromo-6-fluorophenol: To a solution of 4-bromo-2-fluoro-6-nitrophenol (15 g, 0.072 mol, 1.0 eq.) in ethanol (750 mL) was added tin(II)chloride hydrate (68.25 g, 0.36 mol, 5.0 eq.) in one portion. The mixture was stirred at 80° C. for 2 h and reaction was monitored by TLC. Reaction mixture was allowed to come to ambient temperature and poured into ice. The pH was adjusted to 7-8 using aqueous NaOH solution (5 N). Aqueous layer was extracted in ethyl acetate (600 mL×2). The combined organic phases were dried over anhydrous Na₂SO₄ and concentrated under reduced pressure to afford crude, which was used for the next step without any further purification. LCMS: 206 [M+H]⁺, 208 [M+H]⁺

Step-2: Synthesis of 4-bromo-2-fluoro-6-(isopropylamino)phenol: To a stirred solution of 2-amino-4-bromo-6-fluorophenol (19.5 g, 94.6 mmol, 1.0 equiv) in DCM (400 mL) was added acetone (8.24 g, 141.9 mmol, 1.5 equiv) followed by addition of acetic acid (28.42 g, 473.3 mmol, 5.0 equiv) at 0° C. The reaction mixture was stirred at same temperature for 10 minutes. To this was added sodium triacetoxyborohydride (40.13 g, 189.3 mmol, 2.0 equiv) at 0° C. Reaction mixture was stirred at same temperature for 1 h. Reaction was monitored by TLC and LCMS. After completion of the reaction, the reaction mixture was quenched with ice cold water (100 mL) and organic phase was extracted. Organic phase was washed with water (3×100 mL), followed by brine (100 mL) wash. Organic phase was dried over anhydrous sodium sulphate, filtered and concentrated under reduced pressure to afford crude, which was used for the next step without any further purification. LCMS: 248 [M+H]⁺, 250 [M+H]⁺

Step-3: Synthesis of 6-bromo-8-fluoro-4-isopropyl-2H-benzo[b][1,4]oxazin-3(4H)-one: To a stirred solution of 4-bromo-2-fluoro-6-(isopropylamino)phenol (24.5 g, 99.59 mmol, 1.0 equiv) in chloroform (500 mL), was added NaHCO₃ (41.5 g, 497.9 mmol, 5.0 equiv) at 0° C., followed by addition of benzyl triethyl ammonium chloride (22.4 g, 99.9 mmol, 1.0 equiv) at same temperature. The reaction mixture was stirred at 0° C. for 5 min. To this chloroacetyl chloride (11.2 g, 99.6 mmol, 1.0 equiv) was added at 0° C. Reaction mixture was stirred at ambient temperature for 1 h. Reaction was monitored by TLC and LCMS. After completion of the reaction, the reaction mixture was quenched ice cold water (100 mL) and organic phase was extracted with DCM (500 mL×2). The combined organic phase was washed with water (3×100 ml) and brine solution (110 mL). Organic phase was dried over anhydrous sodium sulphate, filtered and concentrated under reduced pressure to afford crude. Crude material from two batches (batch size #23 g & 24.5 g) was purified by column chromatography silica gel (100-200 mesh) using ethyl acetate:hexane 0-40% as eluent to afford desired product. LCMS: 288 [M+H]⁺, 290 [M+H]⁺

Step-4: Synthesis of 6-bromo-8-fluoro-4-isopropyl-3,4-dihydro-2H-benzo[b][1,4]oxazine: To a stirred solution of 6-bromo-8-fluoro-4-isopropyl-2H-benzo[b][1,4]oxazin-3(4H)-one (30 g, 104.5 mmol, 1.0 equiv) in THF (600 mL) BH₃.DMS (2M in THF) (209 mL, 418.1 mmol, 4.0 equiv) was added drop wise at 0° C. The reaction mixture was stirred at 80° C. for 1 h. Reaction was monitored by TLC and LCMS. After completion, solvent was concentrated under reduced pressure. Reaction mixture was quenched with saturated sodium bicarbonate solution (100 mL) at 0° C. and extracted with ethyl acetate (200 mL×2). The combined organic phase was washed with water (200 mL) and brine solution (200 mL). Organic phase was dried over anhydrous sodium sulphate, filtered and concentrated under reduced pressure to afford desired product. The crude was used for the next step without any further purification. LCMS: 274 [M+H]⁺, 276 [M+H]⁺

Step-5: Synthesis of 8-fluoro-4-isopropyl-6-(4,4,5,5-tetramethyl-1,3,2-dioxaborolan-2-yl)-3,4-dihydro-2H-benzo[b][1,4]oxazine: To a stirred solution of 6-bromo-8-fluoro-4-isopropyl-3,4-dihydro-2H-benzo[b][1,4]oxazine (17 g, 62.2 mmol, 1.0 equiv) in dioxane (170 mL), was added 4,4,5,5-tetramethyl-2-(4,4,5,5-tetramethyl-1,3,2-dioxaborolan-2-yl)-1,3,2-dioxaborolane (20.5 g, 80.9 mmol, 1.1 equiv) and potassium acetate (18.3 g, 186.8 mmol, 3.0 equiv) at ambient temperature. Reaction mixture was purged under nitrogen for 15 minutes, followed by addition of PdCl₂(dppf) DCM (2.54 g, 3.11 mmol, 0.05 equiv). The mixture was again purged with nitrogen for 5 min. The reaction mixture was heated at 80° C. for 16 h and monitored by TLC and LCMS. After completion of the reaction, dioxane was removed under reduced pressure. Reaction mixture was diluted with water (200 mL) and extracted with ethyl acetate (1000 mL×2). Combined organic layers were washed with water (200 mL×3). Organic phase was dried over anhydrous sodium sulphate, filtered and concentrated under reduced pressure to afford crude. Crude material from two batches (batch size-#17 g) was purified by column chromatography silica gel (#100-200 mesh) using ethyl acetate:hexane 0-20% as eluent to afford desired product. LCMS: 323 [M+H]⁺

Step-6: Synthesis of 6-(2-chloro-5-fluoropyrimidin-4-yl)-8-fluoro-4-isopropyl-3,4-dihydro-2H-benzo[b][1,4]oxazine: To a stirred solution of 2,4-dichloro-5-fluoropyrimidine (7.6 g, 46.7 mmol, 1 equiv) and 8-fluoro-4-isopropyl-6-(4,4,5,5-tetramethyl-1,3,2-dioxaborolan-2-yl)-3,4-dihydro-2H-benzo[b][1,4]oxazine (15 g, 46.7 mmol, 1.0 equiv) in THF:Water (240 mL:160 mL, 20 mL) was added potassium carbonate (12.91 g, 93.4 mmol, 2.0 equiv) at ambient temperature. Reaction mixture was purged under nitrogen for 15 minutes, followed by addition of Pd(PPh₃)₄ (0.530 g, 0.46 mmol, 0.01 equiv). Reaction mixture was again purged under nitrogen for 5 min. The reaction mixture was heated at 80° C. for 6 h. Reaction was monitored by TLC and LCMS. After completion of the reaction, THF was removed under reduced pressure. Reaction mixture was diluted with water (200 mL) and extracted with ethyl acetate (1000 mL×2). Combined organic phase was washed with water (200 mL×3). Organic phase was dried over anhydrous sodium sulphate, filtered and concentrated under reduced pressure to afford crude. Crude material from two batches (batch size 15 g each) was purified by column chromatography silica gel (#100-200) using 0-30% ethyl acetate:hexane as eluent to afford desired product. LCMS: 326[M+H]⁺, ¹H NMR (400 MHz, Chloroform-d) δ ppm 8.43 (d, J=3.5 Hz, 1H) 7.42 (s, 1H) 7.30 (s, 1H) 4.28-4.42 (m, 2H) 4.11-4.21 (m, 1H) 3.23-3.37 (m, 2H) 1.23 (d, J=6.6 Hz, 6H).

Step-7: Synthesis of tert-butyl 4-(3-nitrophenyl)-1,4-diazepane-1-carboxylate: To a solution of 1-bromo-3-nitrobenzene (300 mg, 1.49 mmol, 1.0 equiv) in dioxane (10 mL), was added tert-butyl 1,4-diazepane-1-carboxylate (597 mg, 2.98 mmol, 2.0 equiv) and cesium carbonate (972 mg, 2.98 mmol, 2 equiv). The reaction mixture was purged with nitrogen gas for 15 min., followed by the addition of Pd₂(dba)₃ (55 mg, 0.059 mmol, 0.04 equiv) and Xantphos (52 mg, 0.089 mmol, 0.06 equiv). The resultant reaction mixture was allowed to stir at 100° C. for overnight. Progress of the reaction was monitored by TLC and LCMS. After completion of the reaction, the reaction mixture was diluted with water (50 mL) and extracted with ethyl acetate (150 mL). Organic layer was washed with water (100 mL) and brine (100 mL). Organic layer was dried over anhydrous sodium sulphate and concentrated under reduced pressure to obtain crude compound, which was purified by normal phase combi flash to obtain desired product. LCMS: 322 [M+H]⁺

Step-8: Synthesis of tert-butyl 4-(3-aminophenyl)-1,4-diazepane-1-carboxylate: To a stirred solution of tert-butyl 4-(3-nitrophenyl)-1,4-diazepane-1-carboxylate (200 mg, 0.62 mmol, 1.0 equiv) in in methanol (10 mL), was added Pd/C (20% w/w) (40 mg) under H₂. The resultant reaction mixture was allowed to stir at RT for 4 h. Progress of the reaction was monitored by LCMS. After completion of the reaction, the mixture was passed through celite bed and the filtrate was concentrated under reduced pressure to obtain crude, which was used for the next step without any further purification. LCMS: 292 [M+H]⁺

Step-9: Synthesis of tert-butyl 4-(3-((5-fluoro-4-(8-fluoro-4-isopropyl-3,4-dihydro-2H-benzo[b][1,4]oxazin-6-yl)pyrimidin-2-yl)amino)phenyl)-1,4-diazepane-1-carboxylate: To a solution of 6-(2-chloro-5-fluoropyrimidin-4-yl)-8-fluoro-4-isopropyl-3,4-dihydro-2H-benzo[b][1,4]oxazine (100 mg, 0.3 mmol, 1.0 equiv) in dioxane (10 mL), was added tert-butyl 4-(3-aminophenyl)-1,4-diazepane-1-carboxylate (96 mg, 0.33 mmol, 1.1 equiv) and cesium carbonate (147 mg, 0.47 mmol, 1.5 equiv). The reaction mixture was purged with nitrogen gas for 30 min, followed by the addition of palladium acetate (2 mg, 0.006 mmol, 0.02 equiv) and BINAP (8 mg, 0.012 mmol, 0.04 equiv). The resultant reaction mixture was allowed to stir at 100° C. for overnight. Progress of the reaction was monitored by TLC and LCMS. After completion of the reaction, the reaction mixture was diluted with water (30 mL) and extracted with ethyl acetate (100 mL). Organic layer was washed with water (50 mL) and brine (50 mL). Organic layer was dried over anhydrous sodium sulphate and concentrated under reduced pressure to obtain crude compound, which was purified by normal phase combi flash to obtain desired product. LCMS: 581 [M+H]⁺

Step-10: Synthesis of N-(3-(1,4-diazepan-1-yl)phenyl)-5-fluoro-4-(8-fluoro-4-isopropyl-3,4-dihydro-2H-benzo[b][1,4]oxazin-6-yl)pyrimidin-2-amine: tert-butyl 4-(3-((5-fluoro-4-(8-fluoro-4-isopropyl-3,4-dihydro-2H-benzo[b][1,4]oxazin-6-yl)pyrimidin-2-yl)amino)phenyl)-1,4-diazepane-1-carboxylate (120 mg, 0.2 mmol, 1.0 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure to obtain crude, which was purified by reverse phase HPLC to obtain desired product. LCMS: 481 [M+H]⁺, ¹H NMR (DMSO-d₆, 400 MHz): δ 9.42 (s, 1H), 8.54 (d, J=3.5 Hz, 1H), 7.40 (br s, 1H), 7.08-7.22 (m, 2H), 7.03 (t, J=7.7 Hz, 1H), 6.34 (d, J=7.5 Hz, 1H), 4.30 (br s, 2H), 4.12 (d, J=6.6 Hz, 1H), 3.51 (br s, 2H), 3.44 (br s, 2H), 3.32 (br s, 2H), 2.85 (br s, 2H), 2.64 (br s, 2H), 1.90 (s, 1H), 1.79 (br s, 2H), 1.01-1.30 (m, 6H).

### Example-2: Synthesis of N-(4-(1,4-diazepan-1-yl)pyridin-2-yl)-S-fluoro-4-(8-fluoro-4-isopropyl-3,4-dihydro-2H-benzo[b][1,4]oxazin-6-yl)pyrimidin-2-amine. (Compound No. 2)

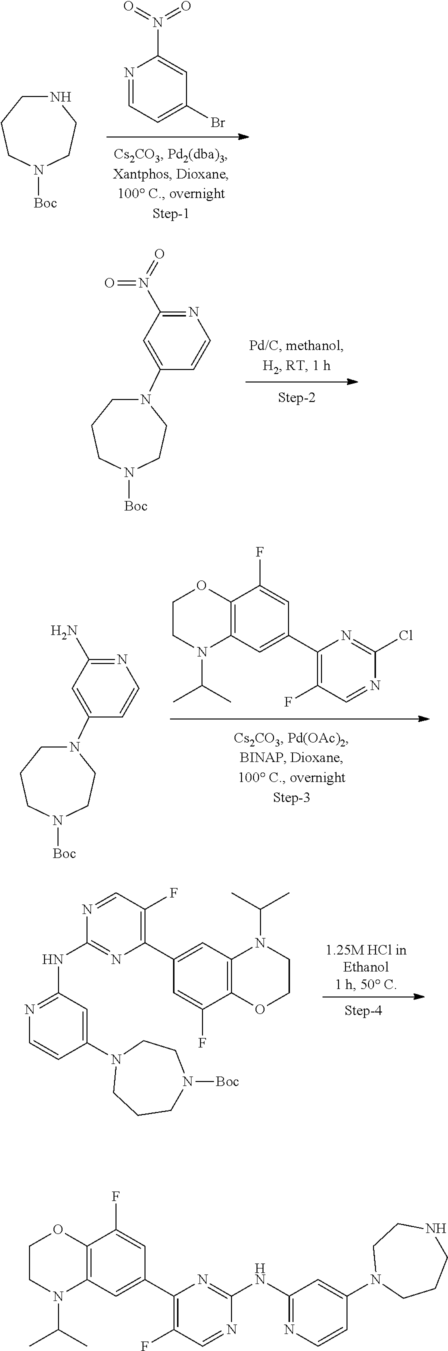

Step-1: Synthesis of tert-butyl 4-(2-nitropyridin-4-yl)-1,4-diazepane-1-carboxylate: To a solution of 4-bromo-2-nitropyridine (300 mg, 1.89 mmol, 1.0 equiv) in dioxane (10 mL), was added tert-butyl 1,4-diazepane-1-carboxylate (456 mg, 2.27 mmol, 1.2 equiv) and cesium carbonate (924 mg, 2.83 mmol, 1.5 equiv). The reaction mixture was purged with nitrogen gas for 15 min, followed by the addition of palladium acetate (17 mg, 0.075 mmol, 0.04 equiv) and Xantphos (66 mg, 0.11 mmol, 0.06 equiv). The resultant reaction mixture was allowed to stir at 100° C. for overnight. Progress of the reaction was monitored by TLC and LCMS. After completion of the reaction, the reaction mixture was diluted with water (50 mL) and extracted with ethyl acetate (150 mL). Organic layer was washed with water (100 mL) and brine (100 mL). Organic layer was dried over anhydrous sodium sulphate and concentrated under reduced pressure to obtain crude compound, which was purified by normal phase combi flash to obtain crude compound, which was purified by normal phase combi flash to obtain desired product. LCMS: 323 [M+H]⁺

Step-2: Synthesis of tert-butyl 4-(2-aminopyridin-4-yl)-1,4-diazepane-1-carboxylate: To a stirred solution of tert-butyl 4-(2-nitropyridin-4-yl)-1,4-diazepane-1-carboxylate (200 mg, 0.62 mmol, 1.0 equiv) in methanol (10 mL), was added Pd/C (20% w/w)(40 mg) under H₂ atm. The resultant reaction mixture was allowed to stir at RT for 4 h. Progress of the reaction was monitored by LCMS. After completion of the reaction, the mixture was passes through celite bed and the filtrate was concentrated under reduced pressure to obtain crude, which was used for the next step without any further purification. LCMS: 293 [M+H]⁺

Step-3: Synthesis of tert-butyl 4-(2-((5-fluoro-4-(8-fluoro-4-isopropyl-3,4-dihydro-2H-benzo[b][1,4]oxazin-6-yl)pyrimidin-2-yl)amino)pyridin-4-yl)-1,4-diazepane-1-carboxylate: To a solution of 6-(2-chloro-5-fluoropyrimidin-4-yl)-8-fluoro-4-isopropyl-3,4-dihydro-2H-benzo[b][1,4]oxazine (100 mg, 0.3 mmol, 1.0 equiv) in dioxane (10 mL), was added tert-butyl 4-(2-aminopyridin-4-yl)-1,4-diazepane-1-carboxylate (96 mg, 0.33 mmol, 1.1 equiv) and cesium carbonate (147 mg, 0.47 mmol, 1.5 equiv). The reaction mixture was purged with nitrogen gas for 30 min, followed by the addition of palladium acetate (2 mg, 0.006 mmol, 0.02 equiv) and BINAP (8 mg, 0.012 mmol, 0.04 equiv). The resultant reaction mixture was allowed to stir at 100° C. for overnight. Progress of the reaction was monitored by TLC and LCMS. After completion of the reaction, the reaction mixture was diluted with water (30 mL) and extracted with ethyl acetate (100 mL). Organic layer was washed with water (50 mL) and brine (50 mL). Organic layer was dried over anhydrous sodium sulphate and concentrated under reduced pressure to obtain crude compound, which was purified by normal phase combi flash to obtain desired product. LCMS: 582 [M+H]⁺

Step-4: Synthesis of N-(4-(1,4-diazepan-1-yl)pyridin-2-yl)-5-fluoro-4-(8-fluoro-4-isopropyl-3,4-dihydro-2H-benzo[b][1,4]oxazin-6-yl)pyrimidin-2-amine: tert-Butyl 4-(2-((5-fluoro-4-(8-fluoro-4-isopropyl-3,4-dihydro-2H-benzo[b][1,4]oxazin-6-yl)pyrimidin-2-yl)amino)pyridin-4-yl)-1,4-diazepane-1-carboxylate (120 mg, 0.2 mmol, 1 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure to obtain crude, which was purified by reverse phase HPLC to obtain desired product. LCMS: 482 [M+H]⁺, ¹H NMR (DMSO-d₄, 400 MHz): δ 10.21 (br s, 1H), 8.65 (br s, 1H), 7.94 (d, J=8.3 Hz, 2H), 7.71 (d, J=7.9 Hz, 2H), 7.60 (br s, 1H), 7.44 (br s, 1H), 7.18 (d, J=12.3 Hz, 1H), 4.31 (br s, 2H), 4.16 (br s, 1H), 3.39 (m, 2H), 3.17 (br s, 1H), 2.99 (br s, 2H), 2.92 (br s, 2H), 2.09 (br s, 1H), 1.54 (br s, 2H), 1.36 (br s, 2H), 1.20 (d, J=6.1 Hz, 6H).

### Example-3: Synthesis of N—(S-((1-ethylpiperidin-4-yl)methoxy)pyridin-2-yl)-5-fluoro-4-(8-fluoro-4-isopropyl-3,4-dihydro-2H-benzo[b][1,4]oxazin-6-yl)pyrimidin-2-amine. (Compound No. 3)

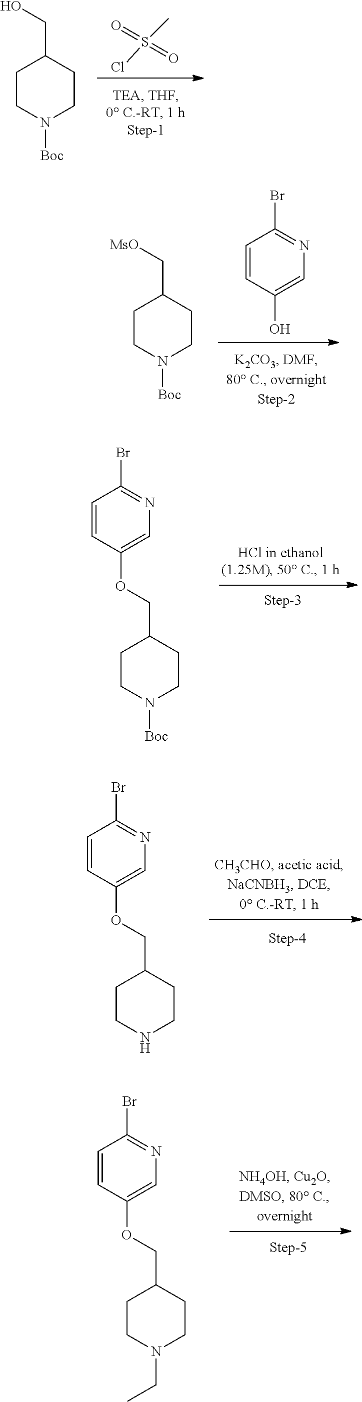

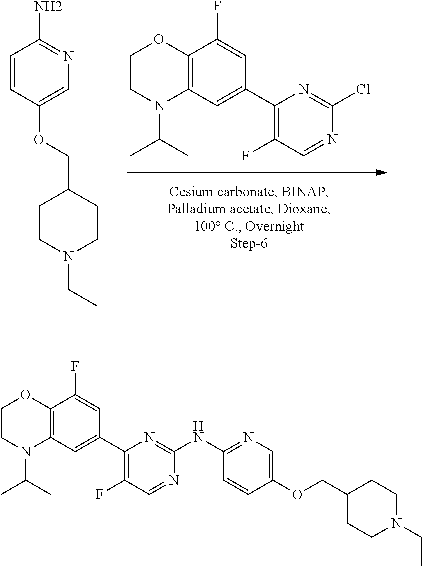 [SMILES: CCN1CCC(COc2ccc(N)nc2)CC1 ; CC(C)N1CCOc2c(F)cc(-c3nc(Cl)ncc3F)cc21 ; CCN1CCC(COc2ccc(Nc3ncc(F)c(-c4cc(F)c5c(c4)N(C(C)C)CCO5)n3)nc2)CC1]

Step-1: Synthesis of tert-butyl 4-(((methylsulfonyl)oxy)methyl)piperidine-1-carboxylate: To a stirred solution of tert-butyl 4-(hydroxymethyl)piperidine-1-carboxylate (5000 mg, 23.2 mmol, 1.0 equiv) in THF (50 mL), was added TEA (6.5 mL, 46.4 mmol, 2.0 equiv). Cooled the reaction mixture to 0° C. followed by the addition of mesyl chloride (2.2 mL, 27.9 mmol, 1.2 equiv). Raised the temperature to RT and the resultant reaction mixture was allowed to stir for 1 h. Progress of the reaction was monitored by HNMR. After completion of the reaction, diluted with water (100 mL) and extracted with EtOAc (150 mL). Organic layer was washed with water (100 mL) and brine solution (100 mL). Organic layer was dried over anhydrous sodium sulphate and concentrated under reduced pressure to obtain crude, which was used for the next step without any further purification. ¹H NMR (Chloroform-d, 400 MHz): δ 4.15 (br s, 1H), 4.06 (s, 1H), 3.01 (s, 3H), 2.71 (t, J=12.5 Hz, 2H), 1.80-2.00 (m, 1H), 1.74 (d, J=12.3 Hz, 1H), 1.50-1.61 (m, 2H), 1.35-1.50 (m, 9H), 1.07-1.31 (m, 2H), 0.83 (br s, 1H).

Step-2: Synthesis of tert-butyl 4-(((6-bromopyridin-3-yl)oxy)methyl)piperidine-1-carboxylate: To a stirred solution of tert-butyl 4-(((methylsulfonyl)oxy)methyl)piperidine-1-carboxylate (1000 mg, 5.7 mmol, 1 equiv) in DMF (10 mL), was added K₂CO₃ (1573 mg, 11.4 mmol, 2 equiv) and 6-bromopyridin-3-ol (2032 mg, 6.9 mmol, 1.2 equiv). The resultant reaction mixture was allowed to stir at 80° C. for overnight. Progress of the reaction was monitored by LCMS. After completion of the reaction, diluted with water (100 mL), solid observed was filtered and dried under vacuum to obtain crude, which was used for the next step without any further purification. LCMS: 371 [M+H]⁺, 373 [M+H]⁺

Step-3: Synthesis of 2-bromo-5-(piperidin-4-ylmethoxy)pyridine: tert-butyl 4-(((6-bromopyridin-3-yl)oxy)methyl)piperidine-1-carboxylate (2000 mg, 5.4 mmol, 1.0 equiv) was taken in 1.25 M HCl in ethanol (10 mL) and the resultant reaction mixture was allowed to stir at 50° C. for 1 h. Progress of the reaction was monitored by LCMS. After completion of the reaction, solvent was removed under reduced pressure to obtain crude as HCl salt, which was used for the next step without any further purification. LCMS: 271 [M+H]⁺, 273 [M+H]⁺

Step-4: Synthesis of 2-bromo-5-((1-ethylpiperidin-4-yl)methoxy)pyridine: To a stirred solution of 2-bromo-5-(piperidin-4-ylmethoxy)pyridine (500 mg, 1.6 mmol, 1.0 equiv) in DCE (5 mL), was added acetaldehyde (40% in water) (0.3 mL, 4.9 mmol, 3.0 equiv) and acetic acid (0.5 mL, 8.0 mmol, 5.0 equiv). The reaction mixture was allowed to stir at RT for 1 h. The reaction mixture was cooled to 0° C. NaCNBH₃ (309 mg, 4.9 mmol, 3.0 equiv) was added to above mixture and raise the temperature to RT. The reaction mixture was allowed to stir at RT for 1 h. Progress of the reaction was monitored by LCMS. After completion of the reaction, the reaction mixture was diluted with water (50 mL) and extracted with DCM (100 mL). Organic layer was washed with water (50 mL) and brine solution (50 mL). Organic layer was dried over anhydrous sodium sulphate and concentrated under reduced pressure to obtain crude, which was used for the next step without any further purification. LCMS: 299 [M+H]⁺, 301 [M+H]⁺

Step-5: Synthesis of 5-((1-ethylpiperidin-4-yl)methoxy)pyridin-2-amine: To a stirred solution of 2-bromo-5-((1-ethylpiperidin-4-yl)methoxy)pyridine (200 mg, 0.67 mmol, 1.0 equiv) in DMSO (5 mL), was added Cu₂O (10 mg, 0.067 mmol, 0.1 equiv) and NH₄OH (40%) (0.5 mL). The resultant reaction mixture was allowed to stir at 80° C. for overnight. Progress of the reaction was monitored by LCMS. After completion of the reaction, it was diluted with saturated solution of NaOH (20 mL) and extracted with EtOAc (100 mL). Organic layer was washed with water (50 mL) and brine solution (50 mL). Organic layer was dried over anhydrous sodium sulphate and concentrated under reduced pressure to obtain crude, which was used for the next step without any further purification. LCMS: 236 [M+H]⁺

Step-6: Synthesis of N-(5-((1-ethylpiperidin-4-yl)methoxy)pyridin-2-yl)-5-fluoro-4-(8-fluoro-4-isopropyl-3,4-dihydro-2H-benzo[b][1,4]oxazin-6-yl)pyrimidin-2-amine: To a solution of 6-(2-chlor-5-fluoropyrimidin-4-yl)-8-fluoro-4-isopropyl-3,4-dihydro-2H-benzo[b][1,4]oxazine (100 mg, 0.3 mmol, 1.0 equiv) in dioxane (5 mL), was added 5-((1-ethylpiperidin-4-yl)methoxy)pyridin-2-amine (78 mg, 0.33 mmol, 1.1 equiv) and cesium carbonate (147 mg, 0.47 mmol, 1.5 equiv). The reaction mixture was purged with nitrogen gas for 10 min, followed by the addition of palladium acetate (2 mg, 0.006 mmol, 0.02 equiv) and BINAP (8 mg, 0.0012 mmol, 0.04 equiv). The resultant reaction mixture was allowed to stir at 100° C. for overnight. Progress of the reaction was monitored by TLC and LCMS. After completion of the reaction, it was diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by normal phase combi flash to obtain desired product. LCMS: 525 [M+H]⁺, ¹H NMR (MeOH-d₄, 400 MHz): δ 8.40 (d, J=4.4 Hz, 1H), 8.18 (d, J=9.2 Hz, 1H), 7.98 (d, J=2.6 Hz, 1H), 7.50 (s, 1H), 7.40 (dd, J=9.0, 2.9 Hz, 1H), 7.24 (d, J=11.0 Hz, 1H), 4.60 (br s, 2H), 4.27-4.37 (m, 2H), 4.06-4.25 (m, 1H), 3.94 (d, J=5.7 Hz, 2H), 3.25 (br s, 2H), 2.74 (d, J=7.5 Hz, 2H), 2.42 (br s, 2H), 1.83-2.08 (m, 3H), 1.56 (d, J=12.3 Hz, 2H), 1.23-1.29 (m, 6H), 1.19 (br s, 3H).

### Example-4: Synthesis of N—(S-((1-ethylpiperidin-4-yl)methoxy)pyridin-2-yl)-5-fluoro-4-(8-fluoro-4-isopropyl-3,4-dihydro-2H-benzo[b][1,4]oxazin-6-yl)pyridin-2-amine. (Compound No. 4)

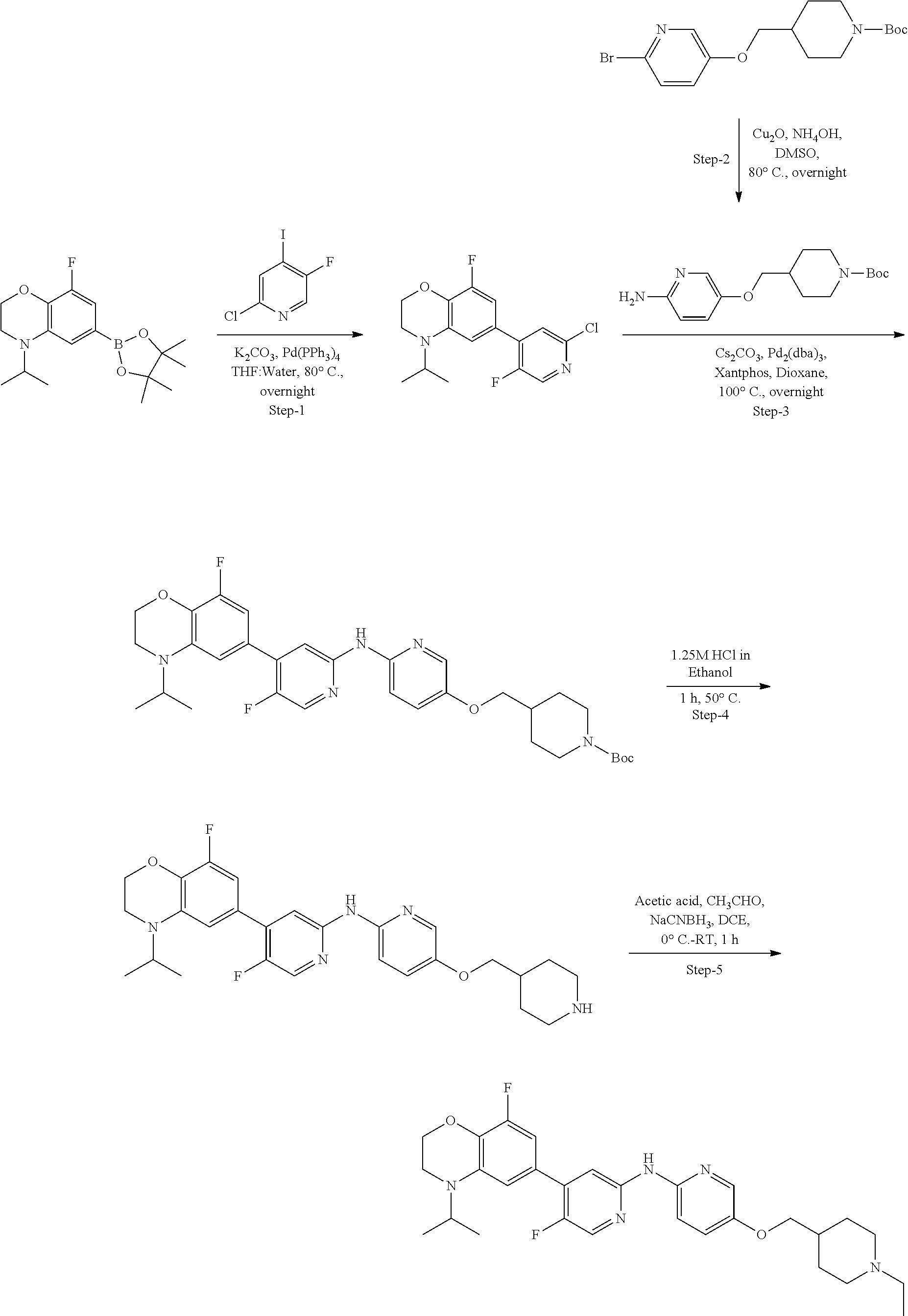

Step-1: Synthesis of 6-(2-chloro-5-fluoropyridin-4-yl)-8-fluoro-4-isopropyl-3,4-dihydro-2H-benzo[b][1,4]oxazine: To a stirred solution of 2-chloro-5-fluoro-4-iodopyridine (600 mg, 2.33 mmol, 1.0 equiv) in THF:water (1:1, 10 mL) was added 8-fluoro-4-isopropyl-6-(4,4,5,5-tetramethyl-1,3,2-dioxaborolan-2-yl)-3,4-dihydro-2H-benzo[b][1,4]oxazine (749 mg, 2.33 mmol, 1.0 equiv), potassium carbonate (644 mg, 4.66 mmol, 2.0 equiv) and Pd(PPh₃)₄ (135 mg, 0.11 mmol, 0.05 equiv). The reaction mixture was allowed to stir at 80° C. for overnight. Progress of the reaction was monitored by TLC and LCMS. After completion of the reaction, the reaction mixture was diluted with water (50 mL) and extracted with ethyl acetate (150 mL). Organic layer was washed with water (50 mL) and brine (50 mL). Organic layer was dried over anhydrous sodium sulphate and concentrated under reduced pressure to obtain crude compound, which was purified by normal phase combi-flash to obtain desired product. LCMS: 325 [M+H]⁺

Step-2: Synthesis of tert-butyl 4-(((6-aminopyridin-3-yl)oxy)methyl)piperidine-1-carboxylate: To a stirred solution of tert-butyl 4-(((6-bromopyridin-3-yl) oxy) methyl) piperidine-1-carboxylate (300 mg, 0.81 mmol, 1.0 equiv) in DMSO (5 mL), was added Cu₂O (12 mg, 0.08 mmol, 0.1 equiv) and NH₄OH (40%) (3 mL). The resultant reaction mixture was allowed to stir at 80° C. for overnight. Progress of the reaction was monitored by LCMS. After completion of the reaction, diluted with saturated solution of NaOH (20 mL) and extracted with EtOAc (100 mL). Organic layer was washed with water (50 mL) and brine solution (50 mL). Organic layer was dried over anhydrous sodium sulphate and concentrated under reduced pressure to obtain crude, which was used for the next step without any further purification. LCMS: 308 [M+H]⁺

Step-3: Synthesis of tert-butyl 4-(((6-((5-fluoro-4-(8-fluoro-4-isopropyl-3,4-dihydro-2H-benzo[b][1,4]oxazin-6-yl)pyridin-2-yl)amino)pyridin-3-yl)oxy)methyl)piperidine-1-carboxylate: To a solution of 6-(2-chloro-5-fluoropyridin-4-yl)-8-fluoro-4-isopropyl-3,4-dihydro-2H-benzo[b][1,4]oxazine (50 mg, 0.15 mmol, 1.0 equiv) in dioxane (5 mL), were added tert-butyl 4-(((6-aminopyridin-3-yl)oxy)methyl)piperidine-1-carboxylate (52 mg, 0.17 mmol, 1.1 equiv) and cesium carbonate (73 mg, 0.23 mmol, 1.5 equiv). The reaction mixture was purged with nitrogen gas for 10 min, followed by the addition of Pd₂(dba)₃ (4 mg, 0.008 mmol, 0.05 equiv) and Xantphos (9 mg, 0.015 mmol, 0.1 equiv). The resultant reaction mixture was allowed to stir at 100° C. for overnight. Progress of the reaction was monitored by TLC and LCMS. After completion of the reaction, the reaction mixture was diluted with water (30 mL) and extracted with ethyl acetate (100 mL). Organic layer was washed with water (50 mL) and brine (50 mL). Organic layer was dried over anhydrous sodium sulphate and concentrated under reduced pressure to obtain crude compound, which was purified by column chromatography to obtain desired product. LCMS: 596 [M+H]⁺

Step-4: Synthesis of 5-fluoro-4-(8-fluoro-4-isopropyl-3,4-dihydro-2H-benzo[b][1,4]oxazin-6-yl)-N-(5-(piperidin-4-ylmethoxy)pyridin-2-yl)pyridin-2-amine: tert-Butyl 4-(((6-((5-fluoro-4-(8-fluoro-4-isopropyl-3,4-dihydro-2H-benzo[b][1,4]oxazin-6-yl)pyridin-2-yl)amino)pyridin-3-yl)oxy)methyl)piperidine-1-carboxylate (70 mg, 0.12 mmol, 1.0 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure and the residue was dried under Lyophiliser to obtain crude, which was used for the next step without any further purification. LCMS: 496 [M+H]⁺

Step-5: Synthesis of N-(5-((1-ethylpiperidin-4-yl)methoxy)pyridin-2-yl)-5-fluoro-4-(8-fluoro-4-isopropyl-3,4-dihydro-2H-benzo[b][1,4]oxazin-6-yl)pyridin-2-amine: To a stirred solution of 5-fluoro-4-(8-fluoro-4-isopropyl-3,4-dihydro-2H-benzo[b][1,4]oxazin-6-yl)-N-(5-(piperidin-4-ylmethoxy)pyridin-2-yl)pyridin-2-amine (50 mg, 0.1 mmol, 1.0 equiv) in DCE (5 mL), was added acetaldehyde (40% in water) (0.02 mL, 0.3 mmol, 3.0 equiv), acetic acid (0.03 mL, 0.5 mmol, 5.0 equiv). The reaction mixture was allowed to stir at RT for 1 h. The reaction mixture was cooled to 0° C. NaCNBH₃ (19 mg, 0.3 mmol, 3.0 equiv) was added to above mixture and raise the temperature to RT. The reaction mixture was allowed to stir at RT for 1 h. Progress of the reaction was monitored by LCMS. After completion of the reaction, the reaction mixture was diluted with water (25 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 524 [M+H]⁺, ¹H NMR (MeOH-d₄, 400 MHz): δ 7.97-8.13 (m, 1H), 7.91 (br s, 1H), 7.62 (d, J=5.7 Hz, 1H), 7.53 (d, J=8.8 Hz, 1H), 7.35 (d, J=6.6 Hz, 1H), 6.87 (s, 1H), 6.70 (d, J=11.0 Hz, 1H), 4.23-4.35 (m, 2H), 4.03-4.18 (m, 1H), 3.94 (d, J=5.3 Hz, 2H), 3.53 (d, J=11.8 Hz, 2H), 3.25 (br s, 2H), 2.98-3.16 (m, 2H), 2.78-2.96 (m, 2H), 2.66 (br s, 1H), 2.09 (d, J=11.8 Hz, 2H), 1.68 (d, J=12.3 Hz, 2H), 1.26-1.40 (m, 3H), 1.02-1.26 (m, 6H).

### Example-5: Synthesis of 5-fluoro-4-(8-fluoro-4-isopropyl-3,4-dihydro-2H-benzo[b][1,4]oxazin-6-yl)-N-(1-(methylsulfonyl)piperidin-4-yl)pyrimidin-2-amine. (Compound No. 5)

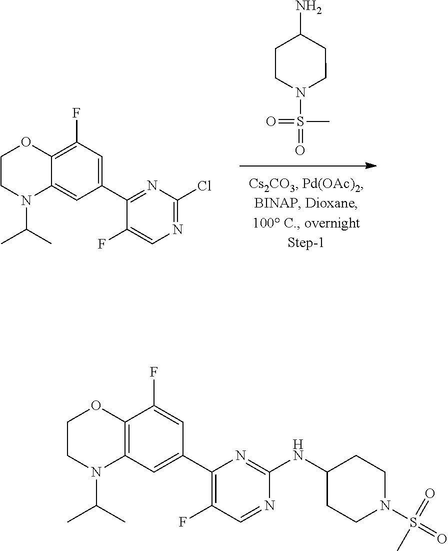 [SMILES: CC(C)N1CCOc2c(F)cc(-c3nc(Cl)ncc3F)cc21 ; CS(=O)(=O)N1CCC(N)CC1 ; CC(C)N1CCOc2c(F)cc(-c3nc(NC4CCN(S(C)(=O)=O)CC4)ncc3F)cc21]

Step-1: Synthesis of 5-fluoro-4-(8-fluoro-4-isopropyl-3,4-dihydro-2H-benzo[b][1,4]oxazin-6-yl)-N-(1-(methylsulfonyl)piperidin-4-yl)pyrimidin-2-amine: To a solution of 6-(2-chloro-5-fluoropyrimidin-4-yl)-8-fluoro-4-isopropyl-3,4-dihydro-2H-benzo[b][1,4]oxazine (100 mg, 0.3 mmol, 1.0 equiv) in dioxane (10 mL), was added 1-(methylsulfonyl)piperidin-4-amine (59 mg, 0.33 mmol, 1.1 equiv) and cesium carbonate (147 mg, 0.47 mmol, 1.5 equiv). The reaction mixture was degassed with nitrogen gas for 30 min, followed by the addition of palladium acetate (2 mg, 0.006 mmol, 0.02 equiv) and BINAP (8 mg, 0.012 mmol, 0.04 equiv). The resultant reaction mixture was allowed to stir at 100° C. for overnight. Progress of the reaction was monitored by TLC and LCMS. After completion of the reaction, diluted with water (30 mL) and extracted with ethyl acetate (100 mL). Organic layer was washed with water (50 mL) and brine solution (50 mL). Organic layer was dried over anhydrous sodium sulphate and concentrated under reduced pressure to obtain crude, which was purified by reverse phase HPLC to obtain desired product. LCMS: 468 [M+H]⁺, ¹H NMR (DMSO-d₆, 400 MHz): δ 8.37 (d, J=4.4 Hz, 1H), 7.33 (br s, 1H), 7.27 (d, J=7.5 Hz, 1H), 7.10 (d, J=11.4 Hz, 1H), 4.21-4.33 (m, 2H), 4.00-4.17 (m, 1H), 3.82 (d, J=7.0 Hz, 1H), 3.55 (d, J=11.8 Hz, 2H), 3.15-3.31 (m, 2H), 2.75-2.94 (m, 5H), 2.00 (d, J=11.0 Hz, 2H), 1.41-1.67 (m, 2H), 1.17 (d, J=6.6 Hz, 6H).

### Example-6: Synthesis of S-fluoro-4-(8-fluoro-4-isopropyl-3,4-dihydro-2H-benzo[b][1,4]oxazin-6-yl)-N-(3-methyl-1-(piperidin-4-yl)-1H-pyrazol-4-yl)pyrimidin-2-amine. (Compound No. 67)

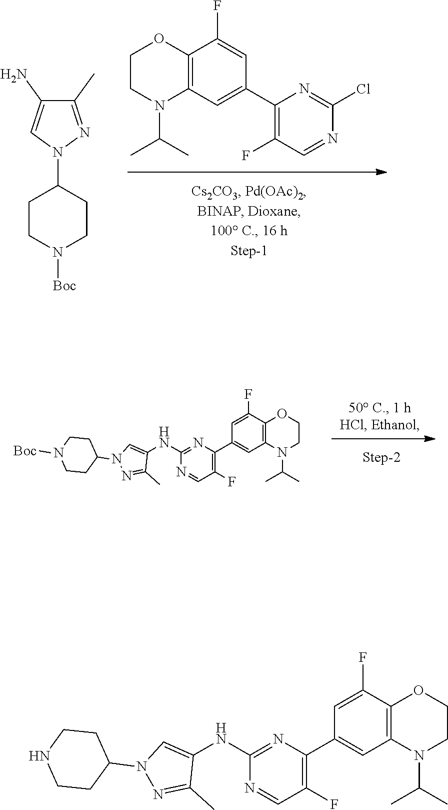

Step-1: Synthesis of tert-butyl 4-(4-((5-fluoro-4-(8-fluoro-4-isopropyl-3,4-dihydro-2H-benzo[b][1,4]oxazin-6-yl)pyrimidin-2-yl)amino)-3-methyl-1H-pyrazol-1-yl)piperidine-1-carboxylate: To a solution of tert-butyl 4-(4-amino-3-methyl-1H-pyrazol-1-yl)piperidine-1-carboxylate (200 mg, 0.714 mmol, 1.0 equiv) in dioxane (5 mL), was added 6-(2-chloro-5-fluoropyrimidin-4-yl)-8-fluoro-4-isopropyl-3,4-dihydro-2H-benzo[b][1,4]oxazine (230 mg, 0.714 mmol, 1.0 equiv) and cesium carbonate (340 mg, 1.07 mmol, 1.5 equiv). The reaction mixture was purged with nitrogen gas for 15 min, followed by the addition of Pd(OAc)₂ (8 mg, 0.036 mmol, 0.05 equiv) and BINAP (44 mg, 0.071 mmol, 0.1 equiv) and again purged with nitrogen for 15 min. The resultant reaction mixture was allowed to stir at 100° C. for overnight. Progress of the reaction was monitored by TLC and LCMS. After completion of the reaction, reaction mixture was filtered through celite bed and washed with ethyl acetate. Volatiles were removed under vacuum and crude was used as such for next reaction. LCMS: 570 [M+H]⁺

Step-2: Synthesis of 5-fluoro-4-(8-fluoro-4-isopropyl-3,4-dihydro-2H-benzo[b][1,4]oxazin-6-yl)-N-(3-methyl-1-(piperidin-4-yl)-1H-pyrazol-4-yl)pyrimidin-2-amine: tert-Butyl 4-(4-((5-fluoro-4-(8-fluoro-4-isopropyl-3,4-dihydro-2H-benzo[b][1,4]oxazin-6-yl)pyrimidin-2-yl)amino)-3-methyl-1H-pyrazol-1-yl)piperidine-1-carboxylate (150 mg, 0.26 mmol, 1 equiv) was taken in 1.25 M HCl in ethanol (5 mL) and the resultant reaction mixture was allowed to stir at 50° C. for 1 h. Progress of the reaction was monitored by LCMS. After completion of the reaction, solvent was removed under reduced pressure and the residue was purified by reverse phase HPLC to obtain desired product. LCMS: 470 [M+H]⁺; ¹H NMR (400 MHz, MeOH-d₄) δ 8.42-8.66 (m, 1H) 8.15-8.28 (m, 1H) 7.69-7.89 (m, 1H) 7.41 (d, J=15.3 Hz, 1H) 7.16 (d, J=11.4 Hz, 1H), 4.28-4.53 (m, 4H) 4.12 (dt, J=12.6, 6.2 Hz, 1H) 3.43-3.63 (m, 2H) 3.03-3.28 (m, 3H) 2.27 (s, 4H) 1.99-2.27 (m, 4H) 1.21 (d, J=6.6 Hz, 6H).

### Example-7: Synthesis of 4-(6-((5-fluoro-4-(8-fluoro-4-isopropyl-3,4-dihydro-2H-benzo[b][1,4]oxazin-6-yl)pyrimidin-2-yl)amino)pyridin-3-yl)piperazin-2-one. (Compound No. 85)

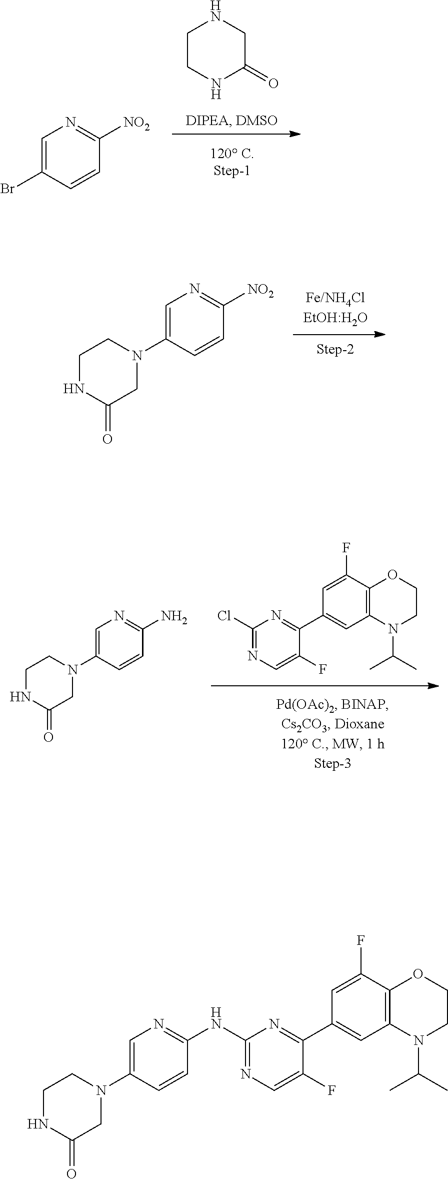 [SMILES: O=C1CNCCN1 ; O=[N+]([O-])c1ccc(Br)cn1 ; O=C1CN(c2ccc([N+](=O)[O-])nc2)CCN1 ; Nc1ccc(N2CCNC(=O)C2)cn1 ; CC(C)N1CCOc2c(F)cc(-c3nc(Cl)ncc3F)cc21 ; CC(C)N1CCOc2c(F)cc(-c3nc(Nc4ccc(N5CCNC(=O)C5)cn4)ncc3F)cc21]

Step-1: Synthesis of 4-(6-nitropyridin-3-yl)piperazin-2-one: To a stirred solution of 5-bromo-2-nitropyridine (6.0 g, 29.55 mmol, 1.0 eq) and piperazin-2-one (3.55 g, 35.46 mmol, 1.2 eq) in DMSO (36 mL), was added DIPEA (18.40 mL, 106.38 mmol, 3.6 eq). The resultant reaction mixture was allowed to stir at 120° C. for 12 h. After completion of the reaction, the precipitate formed was collected by filtration to obtain the desired product. LCMS: 223.3 [M+H]⁺

Step-2: Synthesis of 4-(6-aminopyridin-3-yl)piperazin-2-one: To a stirred solution of 4-(6-nitropyridin-3-yl)piperazin-2-one (4.8 g, 21.60 mmol, 1.0 eq) in ethanol (60 mL):water (60 mL) was added iron (9.65 g, 172.81 mmol, 8.0 eq) and NH₄Cl (11.55 g, 216.0 mmol, 10.0 eq). The resultant reaction mixture was allowed to stir at 80° C. for 2 h. After completion of the reaction, the reaction mixture was filtered over celite, concentrated and purified by silica gel column chromatography to obtain the desired product. LCMS: 193.5 [M+H]⁺

Step-3: Synthesis of 4-(6-((5-fluoro-4-(8-fluoro-4-isopropyl-3,4-dihydro-2H-benzo[b][1,4]oxazin-6-yl)pyrimidin-2-yl)amino)pyridin-3-yl)piperazin-2-one: To a stirred solution of 4-(6-aminopyridin-3-yl)piperazin-2-one (389 mg, 2.02 mmol, 1.0 eq) and 6-(2-chloro-5-fluoropyrimidin-4-yl)-8-fluoro-4-isopropyl-3,4-dihydro-2H-benzo[b][1,4]oxazine (659 mg, 2.02 mmol, 1.0 eq) in dioxane (12 mL) was added Cs₂CO₃ (987 mg, 3.03 mmol, 1.5 eq) at RT. The resulting mixture was purged with nitrogen for 10 min followed by addition of Pd(OAc)₂ (9 mg, 0.040 mmol, 0.02 eq) and BINAP (50 mg, 0.080 mmol, 0.04 eq), again purged with nitrogen for 10 min. The reaction mixture was heated at 100° C. for 1 h under microwave irradiation. The progress of reaction was monitored by LCMS. The reaction mixture was filtered through celite; the residue was washed with EtOAc (10 mL). The filtrate was concentrated and purified by silica gel column chromatography followed by recrystallization in IPA to afford the desired compound. LCMS: 482.5 [M+H]⁺; ¹H NMR (400 MHz, DMSO-d₆): δ ppm 9.71 (br s, 1H) 8.57 (br s, 1H) 8.04 (d, J=9.2 Hz, 2H) 7.33-7.58 (m, 2H) 7.17 (d, J=13.1 Hz, 1H) 4.30 (br s, 2H) 4.15 (br s, 1H) 3.71 (s, 2H), 3.33 (m, 6H) 1.19 (d, J=6.1 Hz, 6H).

### Example-8: Synthesis of 4-(6-((5-fluoro-4-(8-fluoro-4-isopropyl-3,4-dihydro-2H-benzo[b][1,4]oxazin-6-yl)pyrimidin-2-yl)amino)pyridin-3-yl)-1-methylpiperazin-2-one. (Compound No. 86)

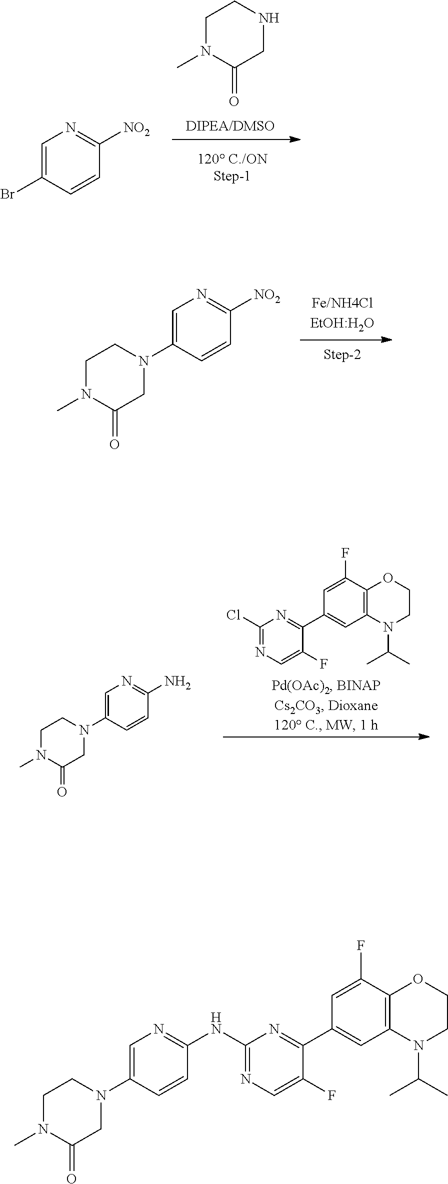

Step-1: Synthesis of 1-methyl-4-(6-nitropyridin-3-yl)piperazin-2-one: To a stirred solution of 5-bromo-2-nitropyridine (1.48 g, 7.30 mmol, 1.0 eq) and 1-methylpiperazin-2-one (1.0 g, 8.76 mmol, 1.2 eq) in DMSO (9 mL), was added DIPEA (4.54 mL, 26.28 mmol, 3.6 eq). The resultant reaction mixture was allowed to stir at 120° C. for 12 h. After completion of the reaction, the precipitate formed was collected by filtration to obtain the desired product. LCMS: 237.4 [M+H]⁺

Step-2: Synthesis of 4-(6-aminopyridin-3-yl)-1-methylpiperazin-2-one: To a stirred solution of 1-methyl-4-(6-nitropyridin-3-yl)piperazin-2-one (1.0 g, 4.23 mmol, 1.0 eq) in ethanol:water (24 mL; 1:1) was added iron (1.89 g, 33.86 mmol, 8.0 eq) and NH₄C₁ (2.26 g, 42.3 mmol, 10.0 eq). The resultant reaction mixture was allowed to stir at 80° C. for 2 h. After completion of the reaction, the reaction mixture was filtered over celite, concentrated to obtain the desired product. LCMS: 193.6 [M+H]⁺

Step-3: Synthesis of 4-(6-((5-fluoro-4-(8-fluoro-4-isopropyl-3,4-dihydro-2H-benzo[b][1,4]oxazin-6-yl)pyrimidin-2-yl)amino)pyridin-3-yl)-1-methylpiperazin-2-one: To a stirred solution of 4-(6-aminopyridin-3-yl)-1-methylpiperazin-2-one (1.0 g, 4.84 mmol, 1.0 eq) and 6-(2-chloro-5-fluoropyrimidin-4-yl)-8-fluoro-4-isopropyl-3,4-dihydro-2H-benzo[b][1.4]oxazine (1.57 g, 4.84 mmol, 1.0 eq) in dioxane (24 mL) was added Cs₂CO₃ (2.36 mg, 7.26 mmol, 1.5 eq) at rt. The resulting mixture was purged with nitrogen for 10 min followed by addition of Pd(OAc)₂ (22 mg, 0.096 mmol, 0.02 eq) and BINAP (121 mg, 0.193 mmol, 0.04 eq), again purged with nitrogen for 10 min. The reaction mixture was heated at 100° C. for 1 h under microwave irradiation. The progress of reaction was monitored by LCMS. The reaction mixture was filtered through celite; the residue was washed with EtOAc (10 mL). The filtrate was concentrated and purified by silica gel chromatography followed by recrystallization in IPA to afford the desired compound. LCMS: 496.6 [M+H]⁺; ¹H NMR (400 MHz, DMSO-d₆): δ ppm 9.72 (s, 1H) 8.57 (d, J=3.9 Hz, 1H) 7.99-8.12 (m, 2H) 7.33-7.56 (m, 2H) 7.16 (s, 1H) 4.30 (t, J=4.1 Hz, 2H) 4.06-4.18 (m, 1H) 3.76 (s, 2H) 3.45 (d, J=3.9 Hz, 4H) 2.90 (s, 3H) 1.19 (d, J=6.6 Hz, 6H).

### Example-9: Synthesis of 8-fluoro-6-(5-fluoro-2-{[5-(1-methylpiperidin-4-yl)pyridin-2-yl]amino}pyrimidin-4-yl)-4-isopropyl-2H-1,4-benzoxazin-3-one. (Compound No. 87)

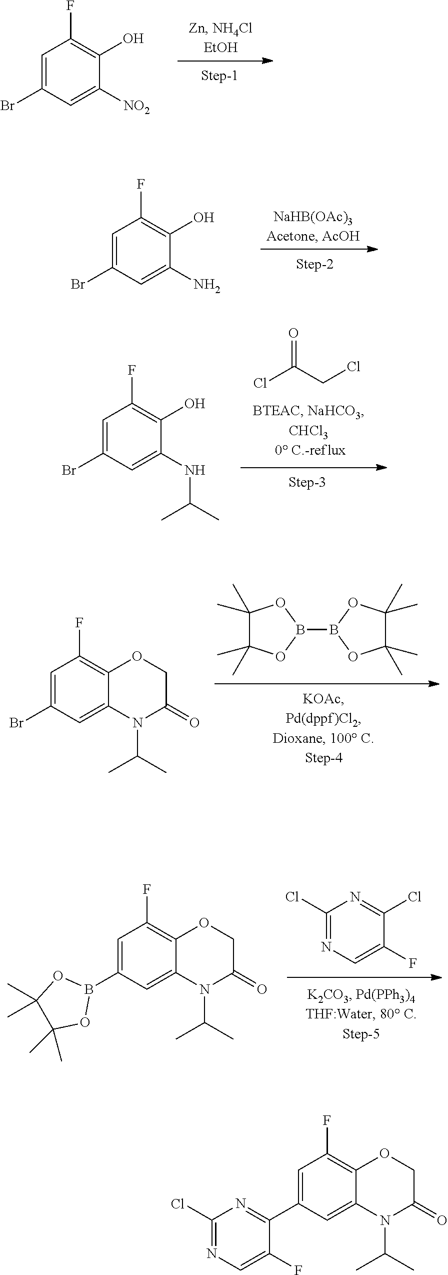

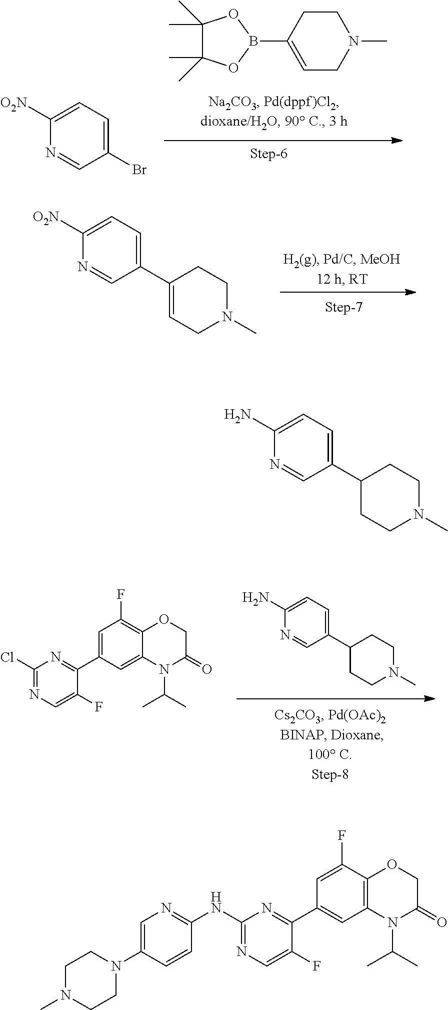

Step-1: Synthesis of 2-amino-4-bromo-6-fluorophenol: To a solution of 4-bromo-2-fluoro-6-nitrophenol (25 g, 105.9 mmol, 1.0 eq) and Zn (34.63 g, 529.5 mmol, 5.0 eq) in EtOH stirred at RT was added a solution of NH₄Cl (56.65 g, 1059 mmol, 10.0 eq) in water dropwise. The reaction mixture was stirred at RT for 1 h. The reaction mixture was filtered, filtrate was collected. EtOH was removed under vacuum. The residue was diluted with H₂O (300 mL) and extracted with ethyl acetate (500 mL×3). The combined organic layer was washed with brine (300 mL×3), then dried over with anhydrous Na₂SO₄. After filtration, the solution was concentrated under vacuum to give the desired product. LCMS: 206 [M+H]⁺

Step-2: Synthesis of 4-bromo-2-fluoro-6-(isopropylamino)phenol: A solution of 2-amino-4-bromo-6-fluorophenol (10 g, 48.5 mmol, 1.0 eq), AcOH (3 mL) in acetone (100 mL) was stirred at RT for 30 min, then the solution was concentrated under vacuum, the residue was dissolved in DCE (100 mL) and sodium triacetoxyborohydride (30.84 g, 145.5 mmol, 3.0 eq) was added, then the reaction mixture was stirred at RT for 2 h. The reaction mixture was diluted with H₂O (200 mL) and extracted with DCM (300 mL×3). The combined organic layer was dried over with anhydrous Na₂SO₄. After filtration, the solution was concentrated under vacuum, and the crude product was purified using silica gel column chromatography to give the desired product. LCMS: 248 [M+H]⁺

Step-3: Synthesis of 6-bromo-8-fluoro-4-isopropyl-2H-1,4-benzoxazin-3-one: To a stirred solution of 4-bromo-2-fluoro-6-(isopropylamino)phenol (2.5 g, 10.1 mmol, 1.0 eq) in CHCl₃ (20 mL) at 0° C. was added NaHCO₃(3388 mg, 40.3 mmol, 4.0 eq) followed by addition of benzyltriethylammonium chloride (2296 mg, 10.1 mmol, 1.0 eq) at 0° C. The reaction mixture was stirred at 0° C. for 5 min. To this was added 2-chloroacetyl chloride (1139 mg, 10.1 mmol, 1.0 eq) in CHCl₃ (5 ml) at 0° C. The reaction mixture was stirred at 0° C. for 1 h, then at 60° C. for 16 h. After the reaction was complete, the reaction was quenched by saturated Na₂CO₃ solution (30 mL) and extracted with DCM (3×40 mL). The combined organic layers were washed with water and dried with Na₂SO₄, and the solvent was removed under reduced pressure. The crude residue was purified by silica gel column chromatography to afford the desired product. LCMS: 288 [M+H]⁺

Step-4: Synthesis of 8-fluoro-4-isopropyl-6-(4,4,5,5-tetramethyl-1,3,2-dioxaborolan-2-yl)-2H-benzo[b][1,4]oxazin-3(4H)-one: To a solution of 6-bromo-8-fluoro-4-isopropyl-2H-1,4-benzoxazin-3-one (500.0 mg, 1.74 mmol, 1.0 eq), 4,4,5,5-tetramethyl-2-(4,4,5,5-tetramethyl-1,3,2-dioxaborolan-2-yl)-1,3,2-dioxaborolane (663 mg, 2.61 mmol, 1.5 eq) and potassium acetate (513 mg, 5.22 mmol, 3.0 eq) in dioxane (10 mL) stirred under nitrogen was added [1,1′-Bis(diphenylphosphino)ferrocene]dichloropalladium(II) (128 mg, 0.17 mmol, 0.1 eq) in one charge. The reaction mixture was stirred under nitrogen at 100° C. for 3 h. Progress of the reaction was monitored by TLC and LCMS. After completion of the reaction, the reaction mixture was then filtered through diatomite and washed with EtOAc. The filtrate was concentrated in vacuo and the crude residue was purified by silica gel column chromatography to afford the desired product. LCMS: 336 [M+H]⁺

Step-5: Synthesis of (6-(2-chloro-5-fluoropyrimidin-4-yl)-8-fluoro-4-isopropyl-2H-benzo[b][1,4]oxazin-3(4H)-one: To a solution of 8-fluoro-4-isopropyl-6-(4,4,5,5-tetramethyl-1,3,2-dioxaborolan-2-yl)-2H-1,4-benzoxazin-3-one (560 mg, 1.67 mmol, 1.0 eq), 2,4-dichloro-5-fluoropyrimidine (419 mg, 2.51 mmol, 1.5 eq) and potassium carbonate (693 mg, 5.01 mmol, 3.0 eq) in THF/H₂O (16 mL; 1:1) stirred under nitrogen at RT was added tetrakis(triphenylphosphine)palladium (193 mg, 0.17 mmol, 0.1 eq) in one charge. The reaction mixture was stirred at 80° C. for 4 h. Progress of the reaction was monitored by TLC and LCMS. After completion of the reaction, the reaction mixture was filtered through celite bed and washed with ethyl acetate (20 mL). The filtrate was concentrated in vacuo and the crude residue was purified by silica gel column chromatography to afford the desired product. LCMS: 340 [M+H]⁺

Step-6: Synthesis of 5-(1-methyl-3,6-dihydro-2H-pyridin-4-yl)-2-nitropyridine: To a solution of 5-bromo-2-nitropyridine (2.0 g, 9.9 mmol, 1.1 eq), 1-methyl-4-(4,4,5,5-tetramethyl-1,3,2-dioxaborolan-2-yl)-3,6-dihydro-2H-pyridine (2.0 g, 8.96 mmol, 1.0 eq) and sodium carbonate (3.8 g, 35.84 mmol, 4.0 eq) in dioxane/H₂O (36 mL; 5:1) stirred under nitrogen was added [1,1′-bis(diphenylphosphino)ferrocene]dichloropalladium(II) (655 mg, 0.89 mmol, 0.1 eq) in one charge. The reaction mixture was stirred at 90° C. for 3 h. After completion of the reaction, the reaction mixture was passed through celite bed, and washed with ethyl acetate (20 mL). The filtrate was concentrated in vacuo and the crude residue was purified by silica gel column chromatography to afford the desired product. LCMS: 220 [M+H]⁺

Step-7: Synthesis of 5-(1-methylpiperidin-4-yl)pyridin-2-amine: To a solution of 5-(1-methyl-3,6-dihydro-2H-pyridin-4-yl)-2-nitropyridine (1.3 g, 0.91 mmol, 1.0 eq) in MeOH/THF (30 mL; 1:1) stirred at RT was added palladium on activated carbon 10% Pd (500 mg). The flask was purged and back-filled with H₂ three times, and then stirred at RT under an H₂ atmosphere for 18 h at 40° C. The mixture was then filtered, and the filtrate was concentrated to give the crude product. LCMS: 192 [M+H]⁺

Step-8: Synthesis of 8-fluoro-6-(5-fluoro-2-{[5-(1-methylpiperidin-4-yl)pyridin-2-yl]amino}pyrimidin-4-yl)-4-isopropyl-2H-1,4-benzoxazin-3-one: To a solution of 6-(2-chloro-5-fluoropyrimidin-4-yl)-8-fluoro-4-isopropyl-2H-1,4-benzoxazin-3-one (180 mg, 0.53 mmol, 1.0 eq), 5-(1-methylpiperidin-4-yl)pyridin-2-amine (122 mg, 0.64 mmol, 1.2 eq) and cesium carbonate (518 mg, 1.59 mmol, 3.0 eq) in dioxane (8 mL) stirred under nitrogen at RT was added 2,2′-bis(diphenylphosphino)-1,1′-binaphthyl (33 mg, 0.053 mmol, 0.1 eq) and palladium (II) acetate (12 mg, 0.053 mmol, 0.1 eq) in one charge. The reaction mixture was stirred at 95° C. for 16 h. Progress of the reaction was monitored by TLC and LCMS. After completion of the reaction, the reaction mixture was filtered through celite bed and washed with ethyl acetate (20 mL). The filtrate was concentrated in vacuo and the crude residue was purified by prep-HPLC (Column: Gemini-C18 150×21.2 mm, 5 um; Flow term: ACN-H₂O (0.1% FA); Gradient: 25-50) to afford the desired product. LCMS: 495 [M+H]⁺; ¹H NMR (400 MHz, CDCl₃) δ 8.50 (s, 1H), 8.44 (d, J=3.6 Hz, 1H), 8.35-8.25 (m, 2H), 8.19 (d, J=2.1 Hz, 1H), 7.87 (s, 1H), 7.70 (dd. J=10.9, 1.6 Hz, 1H), 7.61 (dd, J=8.7, 2.3 Hz, 1H), 4.91 (dt, J=14.1, 7.0 Hz, 1H), 4.66 (s, 2H), 3.43 (d, J=12.2 Hz, 2H), 2.70-2.64 (m, 1H), 2.62 (s, 3H), 2.54 (t, J=11.5 Hz, 2H), 2.22-2.10 (m, 2H), 1.95 (d, J=13.5 Hz, 2H), 1.61 (d, J=7.0 Hz, 6H).

Compounds 6 to 66, 68-84 are synthesized using the general synthetic schemes 1-9 or according to the experimental details as exemplified in Examples 1 to 9 using the appropriate starting materials and reagents.

### Biological Examples

### Example B1. In Vitro Kinase Inhibition IC₅₀ Determination

IC₅₀ values of compounds against CDK4 and CDK6 were determined by luminescence using retinoblastoma as substrate. Kinase assays were performed in kinase buffer (#PV6135, Invitrogen, Life Technologies Grand Island. N.Y.) where total reaction volume was 30 μL/well in 96-well half area white plates (#3693, Costar). One microliter of 25× test compounds at specific concentrations (e.g., final concentration range: 0.1 nM-200 nM) was mixed with 10 μL of 2.5× kinase (5 nM, CDK4 #PR8064 A and CDK6 #PR8422B, Invitrogen) solution and 14 μL of 4× mixed solution with retinoblastoma (1 μM, #12-439, EMD Millipore. Hayward, Calif.) and ATP (25 μM, #V7038, Promega, Madison, Wis.). The plates were covered and incubated for 2H at room temperature. At the end of incubation, 25 μL of stop solution—ADP Glo reagent (#V7002. Promega) was added. After incubation for 45 min at room temperature, 50 μL of detection reagent (##V7002, Promega) was added. Readings were taken at 15 min and 45 min incubation after detection reagent was added in a Synergy Neo Plate reader (BioTek, Winooski, Vt.) at single excitation of 340 nm and Dual emission at 495 nm and 520 nm respectively. The following equations were used in the CDK4 and CDK6 assay data analysis. Percent inhibition (100-% activity) was fitted to the “four-parameter logistic model” in XLfit for determination of IC₅₀ values.

Percent conversion of enzyme=100−{(RLU_{No Drug-No enzyme}*100)/RLU_{No drug+Enzyme}} Equation 1:

Percent conversion at each data point=100−{(RLU_{Average(Drug+enzyme)}*100)/RLU_{No drug+Enzyme}} Equation 2:

Percent Inhibition=100*(% Conversion_{each data point}/% Conversion_{Enzyme}) Equation 3:

IC₅₀ values of compounds against CDK1 (cyclin B) were determined by Z′-LYTE™. These screening assays were performed at Invitrogen Life Technologies (Grand Island, N.Y.) on a low volume NBS, black 384-well plate (#4514, Corning), 0.1 μL of 100× test compound in 100% DMSO (at specific solutions) was mixed with 2.4 μL of Kinase Buffer (50 mM HEPES pH 7.5, 0.01% BRIJ-35, 10 mM MgCl₂, 1 mM EGTA), 5 μL of 2× Kinase (3.5-46.4 ng CDK1/cyclin B)/Peptide (2 μM Ser/Thr 18), and 2.5 μL of 4×ATP solution (34 μM). The plates were shaken for 30 seconds, and incubated for 60 minutes at room temperature. Development Reagent Solution (5 μL of 1:1024 dilution) was added to the plates followed with another 30-second plate shake, and the plates were further incubated at room temperature for one hour. The plates were read on fluorescence plate reader with Dual emission at 445 nm and 520 nm.

IC₅₀ values of compounds against CDK2 (cyclin A) were determined by Z′-LYTE™. These screening assays were performed at Invitrogen Life Technologies (Grand Island, N.Y.) on a low volume NBS, black 384-well plate (#4514. Corning), 0.1 μL of 100× test compound in 100% DMSO (at specific solutions) was mixed with 2.4 μL of Kinase Buffer (50 mM HEPES pH 7.5, 0.01% BRIJ-35, 10 mM MgCl₂, 1 mM EGTA), 5 μL of 2× Kinase (1.22-10.3 ng CDK2/cyclin A)/Peptide (2 μM Ser/Thr 12), and 2.5 μL of 4×ATP solution (31 μM). The plates were shaken for 30 seconds, and incubated for 60 minutes at room temperature. Development Reagent Solution (5 μL of 1:1024 dilution) was added to the plates followed with another 30-second plate shake and the plates were further incubated at room temperature for one hour. The plates were read on fluorescence plate reader with Dual emission at 445 nm and 520 nm.

IC₅₀ values of compounds against CDK5 (p25) are determined by Z′-LYTE™. These screening assays are performed at Invitrogen Life Technologies (Grand Island, N.Y.) on a low volume NBS, black 384-well plate (#4514, Corning), 0.1 μL of 100× test compound in 100% DMSO (at specific solutions) is mixed with 2.4 μL of Kinase Buffer (50 mM HEPES pH 7.5, 0.01% BRIJ-35, 10 mM MgCl₂, 1 mM EGTA), 5 μL of 2× Kinase (0.18-2 ng CDK5/p25)/Peptide (2 μM Ser/Thr 12), and 2.5 μL of 4×ATP solution (17 μM). The plates are shaken for 30 seconds, and incubated for 60 minutes at room temperature. Development Reagent Solution (5 μL of 1:4096 dilution) is added to the plates followed with another 30-second plate shake and the plates are further incubated at room temperature for one hour. The plates are read on fluorescence plate reader with Dual emission at 445 nm and 520 nm.

The following equations were used for Z′-LYTE™ Screening Assay Data Analysis. Percent inhibition (100-% activity) was fitted to the “four-parameter logistic model” in XLfit for determination of IC₅₀ values.

| | Equation |
|---|---|
| Correction for Background Fluorescence | FI_{Sample} − FI_{TCFI Ctl} |
| Emission Ratio (using values corrected for background fluorescence) | $\frac{Coumarin Emission ( 445 nm )}{Fluorescein Emission ( 520 nm )}$ |
| % Phosphorylation (% Phos) | $\{ 1 - \frac{( Emission Ratio \times {F}_{100 \%} - {C}_{100 \%}}{( {C}_{0 \%} - {C}_{100 \%} ) + [ Emission Ratio \times ( {F}_{100 \%} - {F}_{0 \%} ) ]} \} * 100$ |
| % Inhibition | $\{ 1 - \frac{\% {Phos}_{Sample}}{\% {Phos}_{0 \% Inhibition Ctl}} \} * 100$ |
| Z′ (using Emission Ratio values) | $1 - \frac{3 * {Stdev}_{0 \% Phos Ctl} + 3 * {Stdev}_{0 \% Inhibition}}{{Mean}_{0 \% Phos Ctl} - {Mean}_{0 \% Inhibition}}$ |
| Difference Between Data Points | \|% Inhibition_{Point 1} − % Inhibition_{Point 2}\| |
| (single point only) | |
| Development Reaction Interference (DRI) (no ATP control) | $\frac{Emission {Ratio}_{DRI Ctl}}{Emission {Ratio}_{0 \% Phos Ctl}}$ |
| Test Compound Fluorescence Interference (TCFI) | $\frac{{FI}_{TCFI Ctl}}{{FI}_{0 \% Inhibitor Ctl}}$ |
| (check both Coumarin and Fluorescein emissions) | |

FI = Fluorescence intensity
C100% = Average Coumarin Emission signal of the 100% Phos. Control
C0% = Average Coumarin emission signal of the 0% Phos. Control
F100% = Average Fluorescein emission signal of the 100% Phos. Control
F0% = 0 Average Fluorescein emission signal of the 0% Phos. Control
DRI = Development Reaction Interference
TCFI = Test Compound Fluorescence Interference

IC₅₀ values of compounds against CDK7 (cyclin H) are determined by Adapta™ Assay at Invitrogen Life Technologies (Grand Island, N.Y.) where total reaction volume is 10 μL/well in low volume, white 384-well plate (#4512, Corning), 0.100 μL of 100× test compound in 100% DMSO (at specific solutions) is mixed with 2.4 μL of HEPES (30 mM), 2.5 μL of 4×ATP solution (153 μM) and 5 μL of 2× Substrate/Kinase mixture (the 2× CDK7/cyclin H/MNAT1/CDK7/9tide mixture is prepared in 50 mM HEPES pH 7.5, 0.01% BRIJ-35, 10 mM MgCl₂, 1 mM EGTA). The final 10 μL Kinase Reaction consists of 5-38.75 ng CDK7/cyclin H/MNAT1 and 200 μM CDK7/9tide in 32.5 mM HEPES pH 7.5, 0.005% BRIJ-35, 5 mM MgCl₂, 0.5 mM EGTA. The plates are shaken for 30 seconds, centrifuged for 1 min at 1000×g, and incubated for 60 minutes at room temperature, 5 μL of Detection Mix (prepared in TR-FRET Dilution Buffer; the Detection mix consists of EDTA (30 mM), Eu-anti-ADP antibody (6 nM) and ADP tracer, and contains the EC₆₀ concentration of tracer for 5-150 μM ATP) is added to the plates followed with another 30-second plate shake and centrifugation for 1 min at 1000×g, and the plates are further incubated at room temperature for one hour. The plates are read on fluorescence plate reader with Dual emission at 615 nm and 665 nm.

The following equations are used for Adapta™ Assay Data Analysis. The ATP/ADP standard curve is fit to model number 205 (sigmoidal dose-response model) in XLfit. The dose response curve is also curve fit to model number 205.

| | Equation |
|---|---|
| Emission Ratio | $\frac{AF 647 Emission ( 665 nm )}{Europium Emission ( 615 nm )}$ |
| % Conversion | $\{ \frac{{EC}_{50 sc}}{( \frac{{Top}_{50 sc} - {Bottom}_{50 sc}}{Emission {Ratio}_{Sample} - {Bottom}_{50 sc}} ) - 1 ⋀ ( \frac{1}{{Hillslope}_{sc}} )} \} * 100$ |
| % Inhibition | $\{ 1 - \frac{\% {Conversion}_{Sample}}{\% {Conversion}_{0 \% Inhibition Ctrl}} \} * 100$ |
| Difference Between | \|% Inhibition_{Point 1}− % Inhibition_{Point 2}\| |
| Data Points | |
| (single point only) | |
| Test Compound | For each emission wavelength fluorescence interference is flagged for a |
| Interference | compound well that is more than 20% outside the range of the controls. |
| Z′ (using Emission Ratio Values) | $1 - \frac{3 * {Stdev}_{0 \% Conv Ctl} + 3 * {Stdev}_{0 \% Inhibition}}{\vert {Mean}_{0 \% Conv Ctl} - {Mean}_{0 \% Inhibition} \vert} * 100$ |

* SC = Standard Curve

IC₅₀ values of compounds against CDK2 (cyclin E1) are determined by LanthaScreen™ Eu Kinase Binding Assay at Invitrogen Life Technologies (Grand Island, N.Y.) where total reaction volume is 16 μL/well in low volume, white 384-well plates (#784207, Greiner), 0.16 μL of 100× test compound in 100% DMSO (at specific solutions) is mixed with 3.84 μL of Kinase Buffer (50 mM HEPES pH 7.5, 0.01% BRIJ-35, 10 mM MgCl₂, 1 mM EGTA), 8.0 μL of 2× Kinase (2.5 nM)/Antibody (Eu-anti-GST, 2 nM) Mixture and 4.0 μL of 4× Tracer (Tracer 236, 100 nM). The plates are shaken for 30 seconds, and incubated for 60 minutes at room temperature. The plates are read on fluorescence plate reader with Dual emission at 615 nm and 665 nm.

IC₅₀ values of compounds against CDK9 (cyclin K) are determined by LanthaScreen™ Eu Kinase Binding Assay at Invitrogen Life Technologies (Grand Island, N.Y.) where total reaction volume is 16 μL/well in low volume, white 384-well plates (#784207, Greiner), 0.16 μL of 100× test compound in 100% DMSO (at specific solutions) is mixed with 3.84 μL of Kinase Buffer (50 mM HEPES pH 7.5, 0.01% BRIJ-35, 10 mM MgCl₂, 1 mM EGTA), 8.0 μL of 2× Kinase (5 nM)/Antibody (Eu-anti-His, 2 nM) Mixture and 4.0 μL of 4× Tracer (Tracer 236, 100 nM). The plates are shaken for 30 seconds, and incubated for 60 minutes at room temperature. The plates are read on fluorescence plate reader with Dual emission at 615 nm and 665 nm.

IC₅₀ values of compounds against FMS kinase are determined by LanthaScreen™ Eu Kinase Binding Assay at Invitrogen (Life Technologies Grand Island, N.Y.) where total reaction volume is 10 μL in low-volume 384-well plates (#4511, Corning). Serially diluted compounds (3-fold) are incubated with kinase (1.25 nM) for 10 min, following which a mixture of ATP (10 μM) (#A1852, Sigma, St-Louis, Mo.) and fluorescent-PolyGT substrate (200 nM) (#PV3610. Invitrogen, Life Technologies Grand Island, N.Y.) is added and incubated in dark at room temperature for 1H. After 1H, 10 μL stop solution containing Terbium labeled antibody (4 nM) (#PV3529, Invitrogen. Life Technologies Grand Island, N.Y.) and EDTA (#E5134, Sigma, St-Louis, Mo.) (20 mM) in TR-FRET dilution buffer (#PV3574, Invitrogen, Life Technologies Grand Island, N.Y.) is added. Readings are taken in a Synergy Neo Plate reader (BioTek, Winooski, Vt.) at single excitation of 340 nm and Dual emission at 495 nm and 520 nm respectively.

The following equations are used for LanthaScreen Eu Kinase Binding Assay Data Analysis. Percent inhibition (100-% activity) is fitted to the “four-parameter logistic model” in XLfit for determination of IC₅₀ values.

| | Equation |
|---|---|
| Emission Ratio (ER) | $\frac{AF 647 Emission ( 665 nm )}{Europium Emission ( 615 nm )}$ |
| % Displacement | $\{ \frac{{ER}_{0 \% Disp Ctrl} - {ER}_{Sample}}{{ER}_{0 \% Disp Ctrl} - {ER}_{100 \% Disp Ctrl}} \} * 100$ |
| Difference Between | \|% Displacement_{Point 1} − % Displacement_{Point 2}\| |
| Data Points | |
| (single point only) | |
| Test Compound | For each emission wavelength fluorescence interference is flagged for a |
| Interference | compound well that is more than 20% outside the range of the controls. |
| Z′ (using Emission Ratio Values) | $1 - \frac{3 * {Stdev}_{0 \% Disp Ctl} + 3 * {Stdev}_{100 \% Disp Ctl}}{\vert {Mean}_{0 \% Disp Ctl} - {Mean}_{100 \% Disp Ctl} \vert}$ |

IC₅₀ values of compounds against the PI3KS kinase were determined by an assay performed by Reaction Biology Corporation (Malvern, Pa.). Briefly, this assay was conducted in buffer (Tris-HCl 40 mM (pH7.5), Orthovanadate 3 mM, MgCl₂ 20 mM, DTT 2 mM, CHAPS 0.05%, DMSO 1%). PI3K₈ kinase was added to the reaction solution and mixed gently. The test compounds in 100% DMSO (at specific solutions) were mixed with the kinase reaction mixture to achieve the final compounds at pre-defined concentrations (e.g., range—0.5 nM to 100 μM) by Acoustic technology (Echo550; nanoliter range). After incubating for 10 min at room temp, ATP was added into the reaction mixture to initiate the reaction followed by a 30-min incubation at 30° C. After quenching the reaction with ADP-Glo reagent, the plates were incubated for 40 min. The Detection Mixture was added, and the plate was incubated for an additional 30 min. At the end of incubation, luminescence was measured. For data analysis, the luminescence was converted into μM ADP production based on ADP standard curves. The nonlinear regression to obtain the standard curve and IC₅₀ values was performed using GraphPad Prism (GraphPad Software. Inc., San Diego, Calif.).

IC₅₀ values of compounds against CDK12 (cyclin K) are determined by KinaseProfiler™ radiometric protein kinase assay at Eurofins Pharma Discovery (Dundee. UK). Compounds are prepared to 50× final assay concentration in 100% DMSO. This working stock of the compound is added to the assay well as the first component in each reaction. CDK12/Cyclin K is diluted in buffer (20 mM TRIS, 0.2 mM EDTA, 0.1% β-mercaptoethanol, 0.01% Brij-35, 5% glycerol, 1 mg/ml BSA) prior to addition to the reaction mix. CDK12/Cyclin K is incubated with 20 mM Tris/HCl pH 8.5, 0.2 mM EDTA, 300 μM RSRSRSRSRSRSRSR, 10 mM Magnesium acetate and [γ-³³P-ATP] (specific activity and concentration as required). The reaction is initiated by the addition of the Mg/ATP mix. After incubation for 120 minutes at room temperature, the reaction is stopped by the addition of phosphoric acid to a concentration of 0.5%, 10 μl of the stopped reaction is spotted onto a P30 filtermat and washed four times for 4 minutes in 0.425% phosphoric acid and once in methanol prior to drying and scintillation counting. Results are calculated as a percentage of the mean kinase activity in positive control samples. Data are fitted in XLfit for determination of ICs values.

IC₅₀ values of compounds disclosed herein against the kinases listed above are given in Table 2 below.

TABLE 2

| Compound | CDK4 | CDK6 | CDK1/B | CDK2/A | PI3Kδ |
|---|---|---|---|---|---|
| No. | IC₅₀ (nM) | IC₅₀ (nM) | IC₅₀ (nM) | IC₅₀ (nM) | IC₅₀ (nM) |
| 1 | 40 | 53 | ND | ND | ND |
| 2 | >200 | >200 | 2798 | 1228 | >50000 |
| 3 | 5 | 6.5 | 1490 | 207 | >50000 |
| 4 | 7 | 12 | 200 | 75 | >30200 |
| 5 | 2 | 9 | <1 | <1 | >36600 |
| 67 | 16 | 45 | 94 | 45 | ND |
| 85 | 10 | 26 | 4553 | 2471 | ND |
| 86 | 1 | 3 | 1484 | 278 | ND |

ND: Not Detertnined

### Example B2. Determination of Potency of Compounds in Cancer Cell Proliferation Assay as a Single Agent

The effects of test compounds were studied in two breast cancer cell lines of different subtype. The cancer cells (Table 3) were harvested during the logarithmic growth period and counted. Cell concentrations were adjusted to the appropriate number with respective medium and 90 μL cell suspensions were added to 96-well plates. After cells were seeded, the plates were shaken gently to distribute cells evenly and incubated at 37° C., 5% CO₂ on day 1.

TABLE 3
Cell Culture Conditions

| No. | Cell Line | Histopathology | Rb Status | Medium |
|---|---|---|---|---|
| 1 | MCF-7 | Breast adenocarcinoma | Positive | MEM + 10% FBS |
| 2 | DU4475 | Breast carcinoma | Negative | RPMI1640 + 10% |
| | | | | FBS |

Cells were treated with test compounds at 7 to 9 concentrations within a desired concentration range (e.g. 1.1 nM-10 μM) on day 2 by series diluting the test compound stock solution (10 mM in DMSO) with culture medium. Treatment duration was 144H (with a medium change at 72H) for both MCF-7 and DU4475 cells. Cell viability was assessed by Cell Titer-Glo® as recommended by Promega (Cat. No.: G7572), or by resazurin assay (Sigma Aldrich, Cat. No.: R7017) post treatment.

Cell viability data were plotted using GraphPad Prism (GraphPad Software, Inc., San Diego, Calif.). In addition, a nonlinear regression model with a sigmoidal dose response and variable slope within GraphPad Prism was used to calculate the IC₅₀ value of individual test compounds. IC₅₀ values are given in Table 4.

TABLE 4

| Compound | MCF-7 | DU4475 |
|---|---|---|
| No | IC₅₀ (nM) | IC₅₀ (nM) |
| 1 | 252 | 285 |
| 2 | 823 | 1640 |
| 3 | 60 | 655 |
| 4 | 215 | 275 |
| 5 | 525 | 230 |
| 67 | 80 | 130 |
| 85 | 392 | 3610 |
| 86 | 160 | 1140 |

The effects of test compound in a palbociclib-resistant cell line and a parental, non-resistant cell line were compared. The palbociclib-resistant cell line (“MCF-7-PR”) was derived from the parental, non-resistant cell line (MCF-7 breast adenocarcinoma cells) by culture of cells over a period of three months in increasing concentrations of palbociclib, starting from about 350 nM and ending at about 850 nM, the final concentration at which they were then maintained in culture. The MCF-7-PR cells were checked using a cell viability assay to confirm that they had at least 5-fold resistance to palbociclib compared to parental MCF-7 cells, as measured by an increase in the cell viability IC₅₀ values. Assessment of cell viability following treatment with palbociclib or test compound was performed according to the method described above for MCF-7 cells. Results are shown in Table 5.

TABLE 5

| Compound | MCF-7 | MCF-7-PR |
|---|---|---|
| No. | IC₅₀ (nM) | IC₅₀ (nM) |
| 67 | 40 | 72 |

The effects of test compounds are studied in additional cell lines of various histotypes, such as A549 lung adenocarcinoma, HCT-116 colorectal carcinoma, ZR-75-30 breast ductal carcinoma, Hs-578T breast epithelia carcinoma and BT-549 breast ductal carcinoma cells. The cancer cells are harvested during the logarithmic growth period and counted. Cell concentrations are adjusted to the appropriate number with suitable medium, and 90 μL cell suspensions are added to 96-well plates. After cells are seeded, the plates are shaken gently to distribute cells evenly and incubated at 37° C., 5% CO₂ on day 1. Cells are treated with test compounds at typically 7-9 concentrations within a desired concentration range (e.g. 1.5 nM-10 μM) on day 2 by series diluting the test compound stock solution (10 mM in DMSO) with culture medium. Cell viability is assessed by Cell Titer-Glo® as recommended by Promega (Cat. No.: G7572, Promega) typically 48-144H post-treatment, with a medium change as necessary. Cell viability data are plotted using GraphPad Prism (GraphPad Software, Inc., San Diego, Calif.). In addition, a nonlinear regression model with a sigmoidal dose response and variable slope within GraphPad Prism is used to calculate the IC₅₀ value of individual test compounds.

Additional test compounds are studied in the same and/or other cancer cell lines using similar proliferation methods with possible variations in cell seeding densities and/or incubation durations. The cell cycle phase distribution post treatment of test compounds is studied using flow cytometer using DAPI staining. Cellular senescence is evaluated after continuously treating cells for a long time (e.g., 14 days) followed by staining cells lines for Senescence associated-β-galactosidase (SAOCAL).

### Example B3. Determination of pRb Levels

Hypo-phosphorylation of the retinoblastoma protein (pRb) by cyclin D:Cdk4/6 complexes results in active pRb, which is a clinically relevant biomarker associated with CDK4 or CDK6 inhibition. As a confirmatory measure of functional activity of CDK4/6, the Ser780 phosphorylation state of RB1 is assessed. MCF-7 cells are plated at 2.5×10⁵ to 3.0×10⁶ cells/well in 6-well cell-culture plates and incubated at 37° C. for 24H in MEM medium supplemented with 10% FBS. Cells are treated for 24H with a medium containing test compound at various concentrations (e.g., 0.01, 0.1, 1 μM) or with DMSO (≤1%) in duplicate. After incubation period, the medium is removed, and cells are rinsed once with ice-cold PBS and lysed with 0.2 mL of Cell Lysis Buffer containing 1 mM PMSF and Protease Inhibitor. Protein concentration is estimated following Bradford method. The lysis and the pRB measurements are performed following the manufacturer's ELISA kit protocols and buffers (Cell Signaling Technology. Cat. No.: 13016C), pRb inhibition of test compounds is calculated as percentage of vehicle control.

The effects of selected test compounds in additional cancer cell lines on clinically relevant biomarkers associated with CDK4 or CDK6 inhibition (e.g., pRB and thymidine kinase (TK)) is assessed using ELISA or Western Blotting methods with selective antibodies.

### Example B4. Determination of Potency and Combination Effects of Compounds in Cancer Cell Proliferation Assays Using Combination Therapy

Effects of test compounds on cell proliferation is studied in additional cancer cell lines, such as estrogen receptor over-expressing cancer cells, in the combination of another anti-cancer therapy (e.g., an aromatase inhibitor and/or a selective estrogen receptor degrader for breast cancer) using CTG, resazurin and/or Brdu assays. Cells seeded in a 96-well plate are treated with single agents to obtain a dose response curve for each agent. Cells are also treated with combinations of the drugs, based on a matrix generated by combining the two drugs at all different combinations of the doses used in the dose response curves. In place of a combination matrix method, a fixed drug ratio dilution method in which drugs are combined in a fixed ratio of 5 or more dilutions may also be used. The combined treatment effect, such as additive, synergistic, or antagonistic, is determined using the median-effect principle (Chou TC. Cancer Res 2010; 70:440-6), with the combination index (CI) value indicating an additive effect (CI=1), synergism (CI<1), or antagonism (CI>1) in drug combinations.

### Example B5. In Vivo Pharmacology Studies in Xenograft or Syngeneic Models

The anti-tumor activity of test compounds is studied against various human tumor xenograft or syngeneic models in mice for example, in breast cancer tumor models. For breast cancer tumor models, effects of test compounds on Rb-Positive or Rb-Negative tumors as a single agent or in combination with another anti-cancer therapy is determined by evaluating the difference of tumor volume between treatment group against the vehicle control group. The phosphorylation status of serine-780 on Rb is evaluated in tumor tissue and compared with antitumor response in Rb-Positive xenograft model(s). Additional pharmacodynamic end points (e.g., FoxM1, E2F1, c-Myc, and cyclin D1) are studied in tumor tissues collected at various time points post treatment. Induction of senescence is evaluated in tumor samples from various treatment groups by measuring SAβGAL.

### Example B6. In Vivo Pharmacology Study in MC-38 Mouse Model

The therapeutic efficacy of test compound in the treatment of the MC-38 murine colorectal cancer model is evaluated in combination with an anti mPD-1 antibody. Cultured MC-38 cells are harvested and re-suspended in base medium at a density of 1×10⁷ cells/mL with viability greater than 90%. Female C57BL/6 mice are inoculated subcutaneously at the right flank with 1×10⁶ cells in 0.1 mL base medium for tumor development. The mice are stratified into treatment groups and the treatments are started after tumor inoculation when the tumor size reaches, for example, 45-72 mm³ (average tumor size 56 mm³). Tumors are measured using a caliper and tumor volumes calculated using the formula: Tumor volume=(a×b²/2) where ‘b’ is the smallest diameter and ‘a’ is the largest diameter. The treatment groups are, for example: vehicle control, test compound alone, anti mPD-1 alone, and test compound+anti mPD-1 at 10 mice per group. The exact treatment groups, drug dose, and dosing schedule are determined specifically for each study according to standard practice. Tumor growth is monitored, and volume recorded at regular intervals. When the individual tumor of each mouse reaches an approximate end-point (for example, tumor volume >2,000 mm³), the mouse is sacrificed. The tumor growth inhibition (TGI) is calculated by comparing the control group's tumor measurements with the other study groups once the predetermined endpoint is reached in the control group.

Although the foregoing invention has been described in some detail by way of illustration and example for purposes of clarity of understanding, it is apparent to those skilled in the an that certain minor changes and modifications will be practiced in light of the above teaching. Therefore, the description and examples should not be construed as limiting the scope of the invention.

What is claimed is:

1. A compound of the Formula (I): 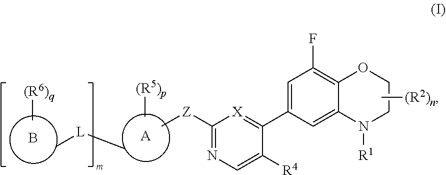
or a salt thereof, wherein:
Z is —NH—, —C(O)NH—, —NH(CO)—, —S(O)₂NH—, or —NHS(O)₂—;
X is N or CR^{a}, wherein R^{a} is hydrogen or —CN;
A is C₃-C₆ cycloalkyl, 4- to 7-membered heterocyclyl, 5- to 7-membered heteroaryl, or C₆ aryl, each of which is optionally substituted by R⁵;
L is a bond, —(CR¹¹R¹²)ᵣ—, —CR¹¹R¹²—O—, —O—, —S—, —S(O)₂—, —C(O)—, —NR¹⁰—, —S(O)₂NR¹⁰—, or NR¹⁰S(O)₂—, wherein r is 1, 2 or 3;
B is hydrogen, C₃-C₁₂ cycloalkyl, or 3- to 12-membered heterocyclyl, wherein the C₃-C₁₂ cycloalkyl and 3- to 12-membered heterocyclyl of B are each independently optionally substituted by R⁶;
R¹ is C₁-C₆ alkyl, C₂-C₆ alkenyl, C₂-C₆ alkynyl, C₃-C₆ cycloalkyl, 3- to 12-membered heterocyclyl, —(C₁-C₃ alkylene)(C₃-C₆ cycloalkyl), or —(C₁-C₃ alkylene)(3- to 12-membered heterocyclyl), each of which is independently optionally substituted by halogen, —OR¹³, —NR¹³R¹⁴, —C(O)R¹³, —CN, C₃-C₈ cycloalkyl, or C₁-C₆ alkyl optionally substituted by oxo, —OH or halogen, provided that when n is 1 and R² is oxo, then R¹ is C₂-C₆ alkyl, C₂-C₆ alkenyl, C₂-C₆ alkynyl, C₃-C₆ cycloalkyl, 3- to 12-membered heterocyclyl, or —(C₁-C₃ alkylene)(C₃-C₆ cycloalkyl), each of which is independently optionally substituted by halogen, —OR¹³, —C(O)R¹³, —CN, C₃-C₈ cycloalkyl, or C₁-C₆ alkyl optionally substituted by oxo, —OH or halogen;
each R² is independently C₁-C₆ alkyl, oxo, —NR¹¹R¹², —CN, —C(O)R¹⁰, —C(O)NR¹¹R¹² or halogen, wherein any two R² groups are independently attached to same carbon or two different carbons;
R⁴ is hydrogen, C₁-C₆ alkyl, C₃-C₆ cycloalkyl, C₁-C₆ haloalkyl, C₁-C₆ alkoxy, C₁-C₆ haloalkoxy, halogen, —CN, or —OH;
each R⁵ is independently C₁-C₆ alkyl, C₂-C₆ alkenyl, C₂-C₆ alkynyl, halogen, oxo, —CN, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), or —(C₁-C₃ alkylene)(3- to 12-membered heterocyclyl), wherein the C₁-C₆ alkyl, C₂-C₆ alkenyl, C₂-C₆ alkynyl, —OR¹⁰, —SR¹⁰, —NR¹¹R¹², —C(O)R¹⁰, —C(O)NR¹¹R¹², —OC(O)NR¹¹R¹², —NR¹⁰C(O)R¹¹, —NR¹⁰C(O)NR¹¹R¹², —S(O)R¹⁰, —S(O)₂R¹⁰, —NR¹⁰S(O)₂R¹¹, —S(O)₂NR¹¹R¹², C₃-C₆ cycloalkyl, —(C₁-C₃ alkylene)OR¹⁰, —(C₁-C₃ alkylene)SR¹⁰, —(C₁-C₃ alkylene)NR¹¹R¹², —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)NR¹⁰S(O)₂NR¹¹R¹², —(C₁-C₃ alkylene)S(O)₂NR¹¹R¹², —(C₁-C₃ alkylene)(C₃-C₆ cycloalkyl), and —(C₁-C₃ alkylene)(3- to 12-membered heterocyclyl) of R⁵ are each independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₈ cycloalkyl, or C₁-C₆ alkyl optionally substituted by oxo, —OH or halogen;
each R⁶ is independently oxo, halogen, or R⁷,
R⁷ is independently hydrogen, C₁-C₆ alkyl, C₂-C₆ alkenyl, C₂-C₆ alkynyl, C₃-C₆ cycloalkyl, 3- to 6-membered heterocyclyl, —OR¹⁰, —NR¹¹R¹², —NR¹⁰C(O)R¹¹, —NR¹⁰C(O)NR¹¹R¹², —S(O)₂R¹⁰, —NR¹⁰S(O)₂R¹¹, —S(O)₂NR¹¹R¹², —C(O)R¹⁰, —C(O)NR¹¹R¹², —(C₁-C₃ alkylene)CN, —(C₁-C₃ alkylene)OR¹⁰, —(C₁-C₃ alkylene)SR¹⁰, —(C₁-C₃ alkylene)NR¹¹R¹², —(C₁-C₃ alkylene)CF₃, —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)S(O)₂NR¹¹R¹², (C₁-C₃ alkylene)(C₃-C₆ cycloalkyl), or —(C₁-C₃ alkylene)(3- to 6-membered heterocyclyl), wherein the C₁-C₆ alkyl, C₂-C₆ alkenyl, C₂-C₆ alkynyl, C₃-C₆ cycloalkyl, 3- to 6-membered heterocyclyl, —OR¹⁰, —NR¹¹R¹², —NR¹⁰C(O)R¹¹, —NR¹⁰C(O)NR¹¹R¹², —S(O)₂R¹⁰, —NR¹⁰S(O)₂R¹¹, —S(O)₂NR¹¹R¹², —C(O)R¹⁰, —C(O)NR¹¹R¹², —(C₁-C₃ alkylene)CN, —(C₁-C₃ alkylene)OR¹⁰, —(C₁-C₃ alkylene)SR¹⁰, —(C₁-C₃ alkylene)NR¹¹R¹², —(C₁-C₃ alkylene)CF₃, —(C₁-C₃ alkylene)C(O)R¹⁰, —(C₁-C₃ alkylene)C(O)NR¹¹R¹², —(C₁-C₃ alkylene)NR¹⁰C(O)R¹¹, —(C₁-C₃ alkylene)NR¹⁰C(O)NR¹¹R¹², —(C₁-C₃ alkylene)S(O)₂R¹⁰, —(C₁-C₃ alkylene)NR¹⁰S(O)₂R¹¹, —(C₁-C₃ alkylene)S(O)₂NR¹¹R¹², (C₁-C₃ alkylene)(C₃-C₆ cycloalkyl), and —(C₁-C₃ alkylene)(3- to 6-membered heterocyclyl) of R⁷ are each independently optionally substituted by halogen, oxo, —OR¹³, —NR¹³R¹⁴, —C(O)R¹³, —CN, —(C₁-C₃ alkylene)OR¹³, —(C₁-C₃ alkylene)NR¹³R¹⁴, —(C₁-C₃ alkylene)C(O)R¹³, C₃-C₅ cycloalkyl, or C₁-C₆ alkyl optionally substituted by oxo, —OH or halogen;
R¹⁰ is independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl or 3- to 6-membered heterocyclyl, wherein the C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, and 3- to 6-membered heterocyclyl of R¹⁰ are each independently optionally substituted by halogen, oxo, —CN, —OR¹⁵, —NR¹⁵R¹⁶, or C₁-C₆ alkyl optionally substituted by halogen, —OH or oxo;
R¹¹ and R¹² are each independently hydrogen, C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl or 3- to 6-membered heterocyclyl, wherein the C₁-C₆ alkyl, C₃-C₆ cycloalkyl, —(C₁-C₃ alkylene)(C₃-C₆ cycloalkyl), C₆-C₁₄ aryl, 5- to 6-membered heteroaryl, and 3- to 6-membered heterocyclyl of R¹¹ and R¹² are each independently optionally substituted by halogen, oxo, —CN, —OR¹⁵, —NR¹⁵R¹⁶ or C₁-C₆ alkyl optionally substituted by halogen, —OH or oxo;
R¹³ and R¹⁴ are each independently hydrogen or C₁-C₆ alkyl, wherein the C₁-C₆ alkyl of R¹³ and R¹⁴ are optionally substituted by halogen, —OR¹⁵, —NR¹⁵R¹⁶, or oxo;
or R¹³ and R¹⁴ are taken together with the atom to which they attached to form a 3- to 6-membered heterocyclyl optionally substituted by halogen, oxo or C₁-C₆ alkyl optionally substituted by halogen or oxo; and
R⁵ and R¹⁶ are each independently hydrogen, C₁-C₆ alkyl optionally substituted by halogen or oxo, C₂-C₆ alkenyl optionally substituted by halogen or oxo, or C₂-C₆ alkynyl optionally substituted by halogen or oxo;
or R⁵ and R¹⁶ are taken together with the atom to which they attached to form a 3- to 6-membered heterocyclyl optionally substituted by halogen, oxo or C₁-C₆ alkyl optionally substituted by oxo or halogen;
p and q are each independently 0, 1, 2 or 3;
m is 0 or 1; and
n is 0, 1, 2, 3 or 4.

2. The compound of claim 1, or a salt thereof, wherein m is 0.

3. The compound of claim 1, or a salt thereof, wherein m is 1.

4. The compound of claim 1, or a salt thereof, wherein the compound is of Formula (I-A):

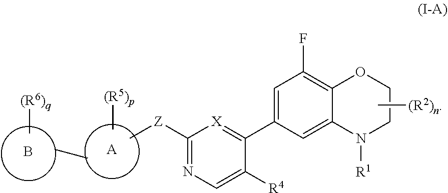

5. The compound of claim 1, or a salt thereof, wherein the compound is of any one of Formula (I-B1) to (I-B20): 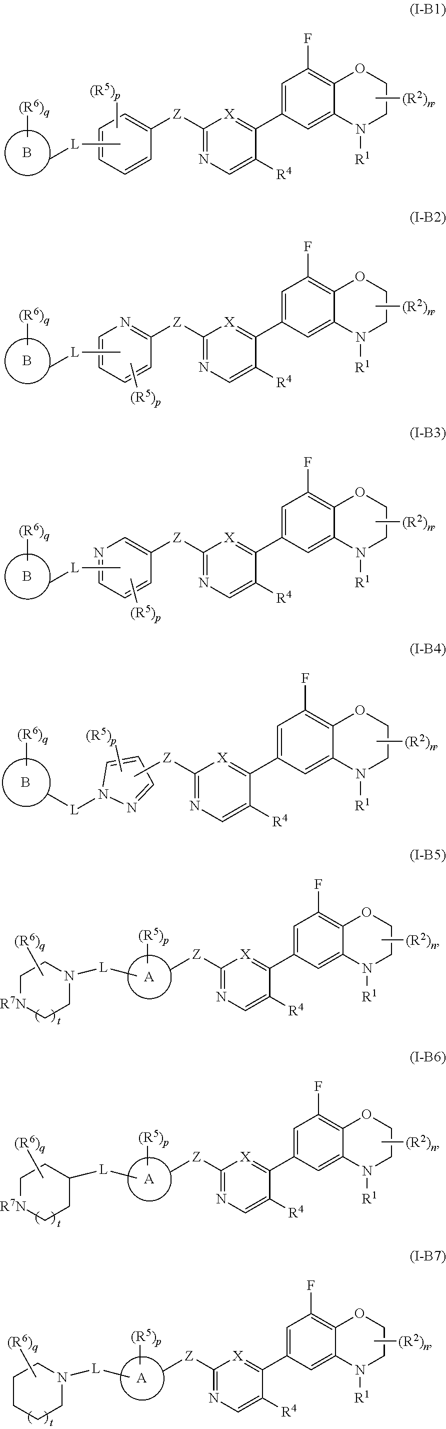 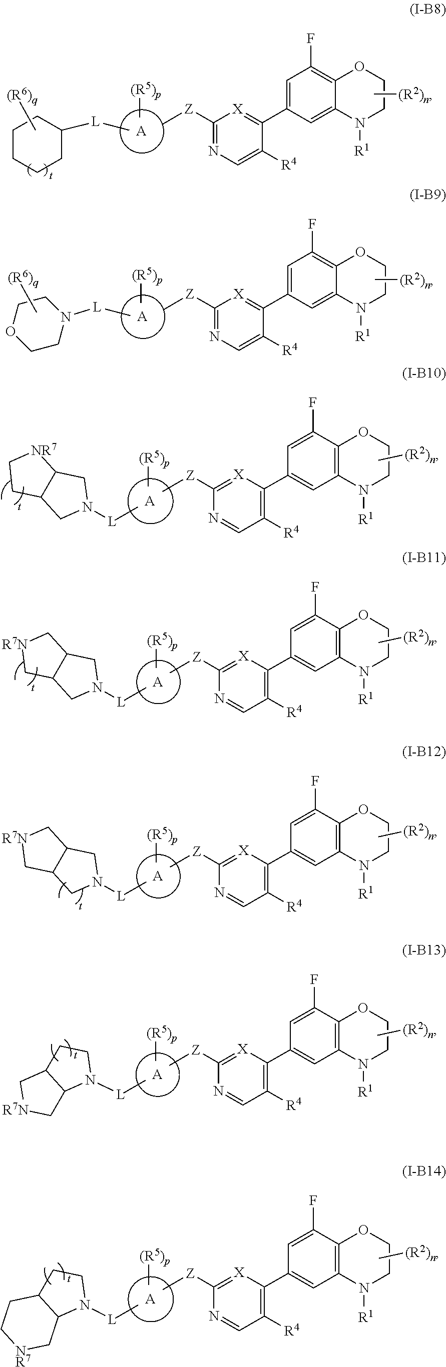 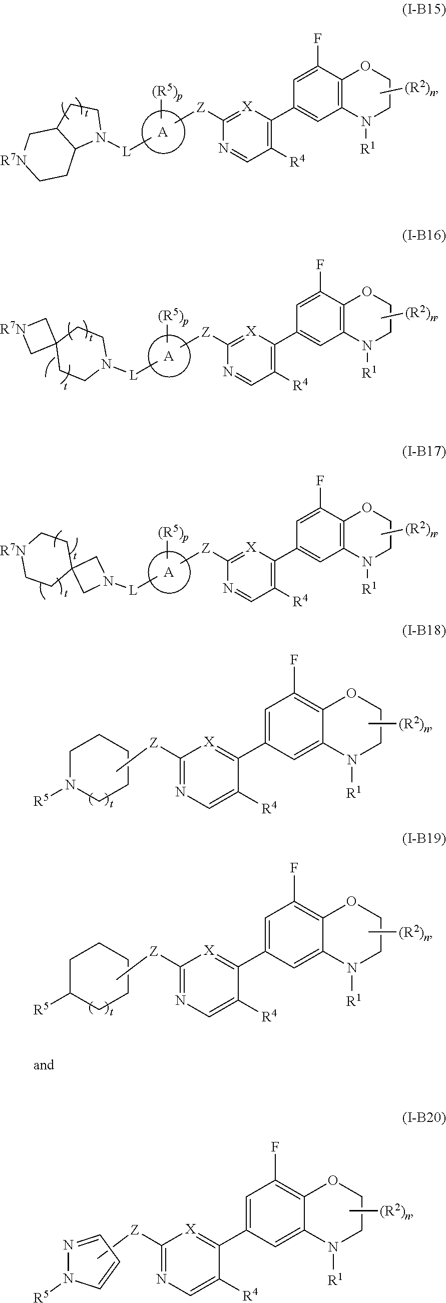
wherein t is 0, 1 or 2.

6. The compound of claim 1 or 5, or a salt thereof, wherein L is a bond.

7. The compound of claim 1 or 5, or a salt thereof, wherein L is —(CR¹¹R¹²)ᵣ.

8. The compound of claim 1 or 5, or a salt thereof, wherein L is —CR¹¹R¹²—O—.

9. The compound of claim 1 or 5, or a salt thereof, wherein L is —O—.

10. The compound of claim 1 or 5, or a salt thereof, wherein L is —S(O)₂—.

11. The compound of claim 1 or 5, or a salt thereof, wherein L is —C(O)—.

12. The compound of claim 1 or 5, or a salt thereof, wherein L is —NR¹⁰—.

13. The compound of claim 1 or 5, or a salt thereof, wherein L is —S(O)₂NR¹⁰—.

14. The compound of claim 1 or 5, or a salt thereof, wherein L is —NR¹⁰S(O)₂—.

15. The compound of any one of claims 1-14, or a salt thereof, wherein A is phenyl, pyridyl, pyrazinyl, piperidinyl, pyrazolyl, or cyclohexyl, each of which is optionally substituted by R⁵.

16. The compound of any one of claims 1-15, or a salt thereof, wherein B is 3- to 12-membered heterocyclyl or C₃-C₁₂ cycloalkyl, each of which is optionally substituted by R⁶.

17. The compound of claim 1, or a salt thereof, wherein the compound is of any one of Formula (I-C₁) to (I-C₄₅): 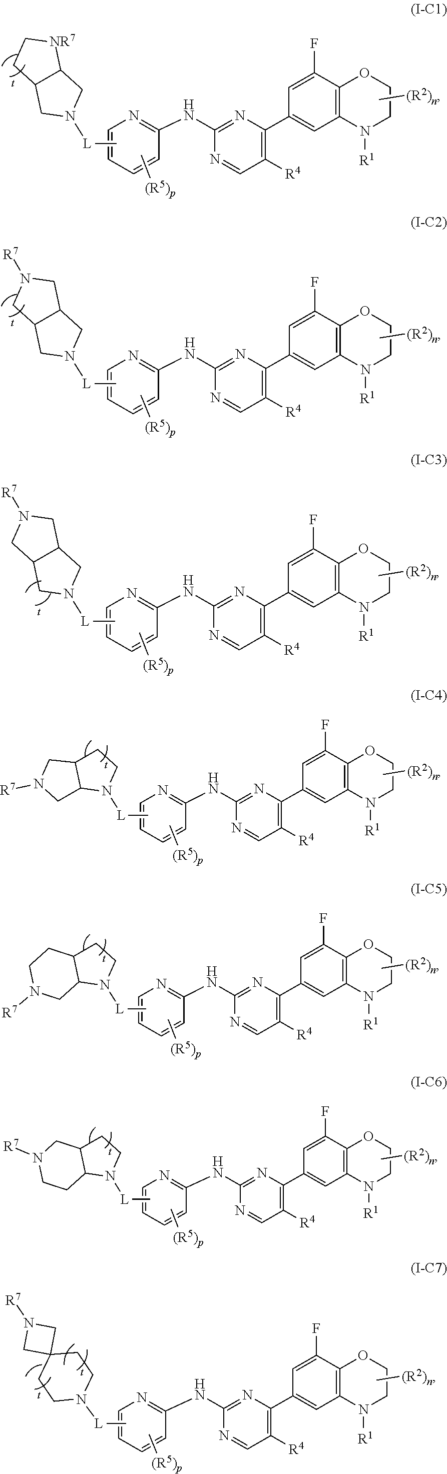 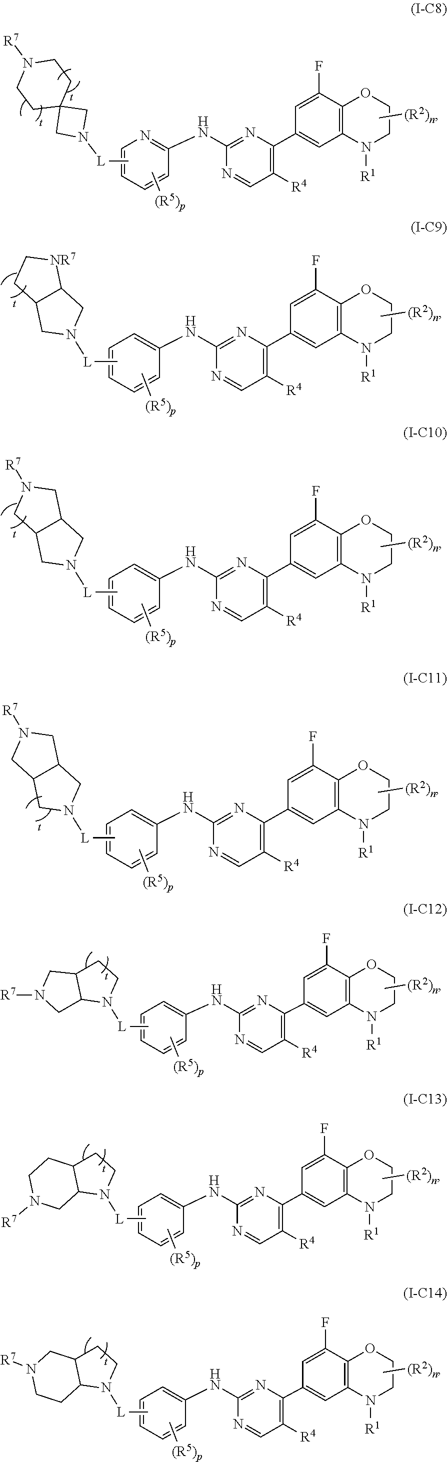 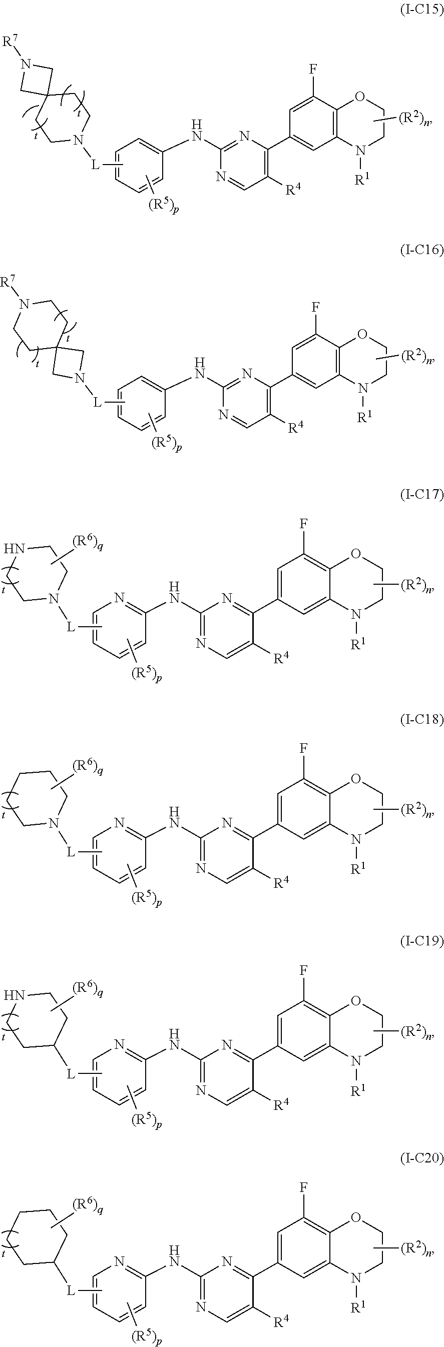 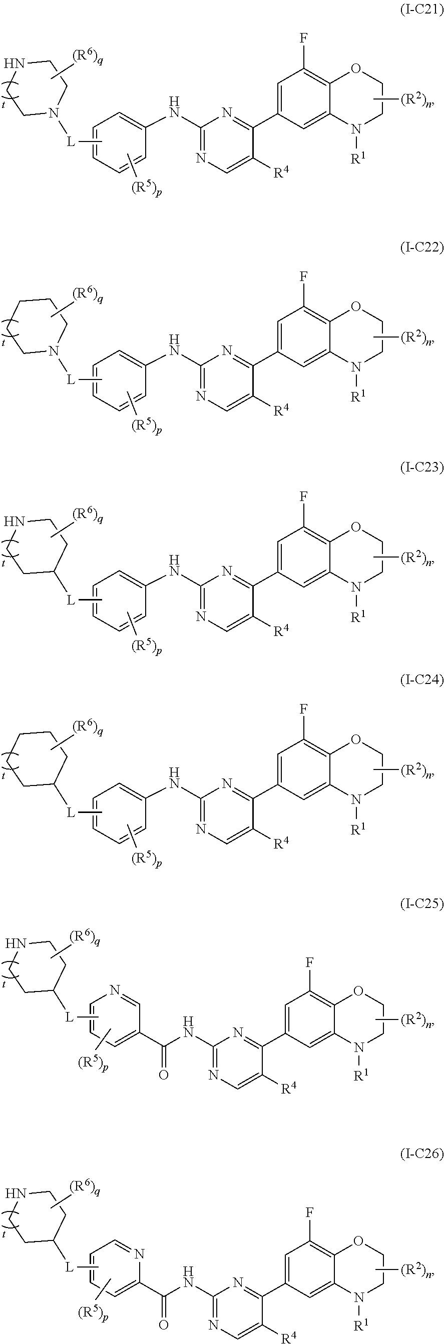 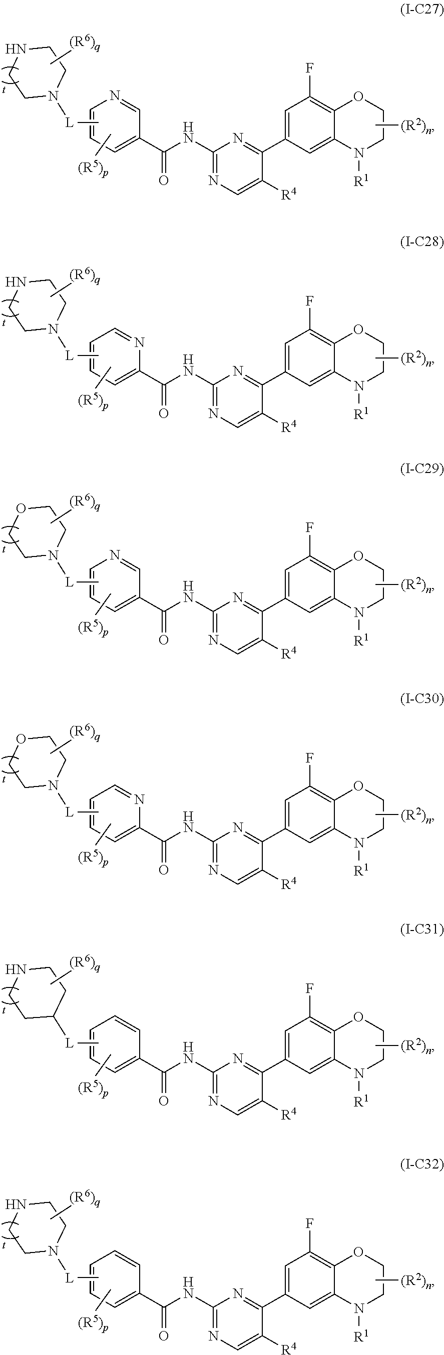 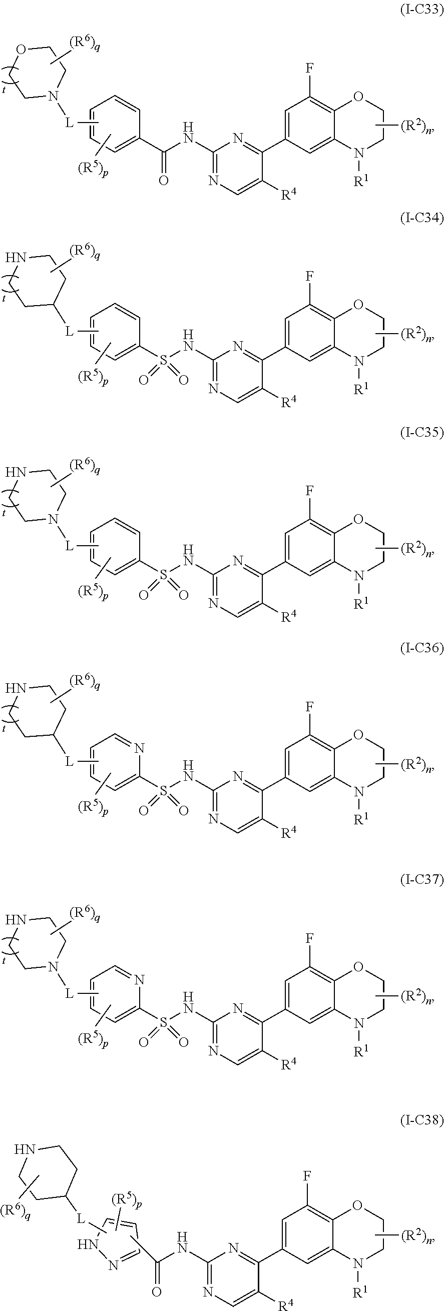 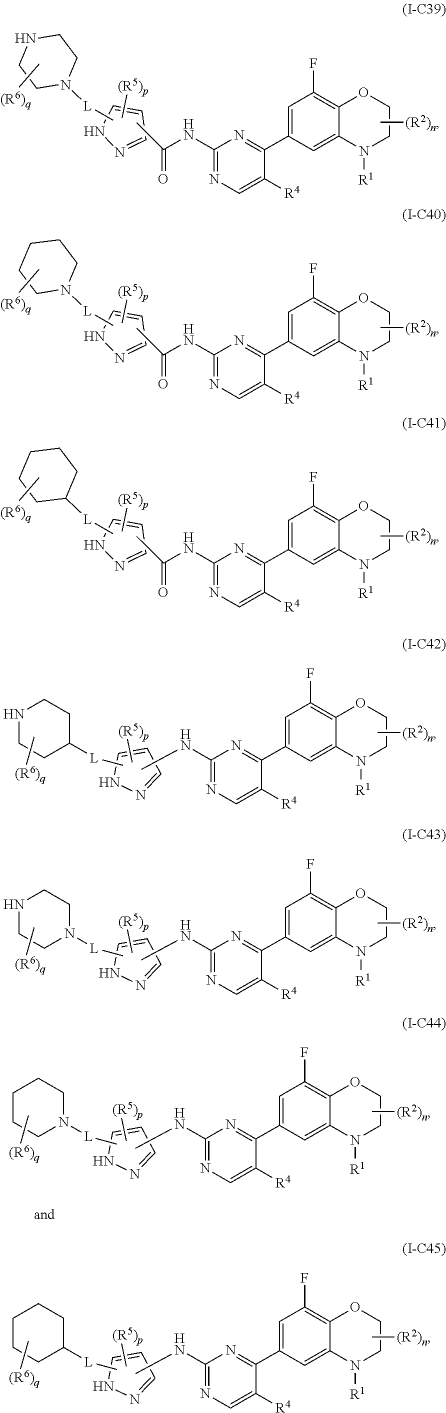
wherein t is 0, 1, 2, or 3.

18. The compound of any one of claims 1-17, or a salt thereof, wherein p is 0 or 1.

19. The compound of any one of claims 1-18, or a salt thereof, wherein each R⁵ is independently C₁-C₆ alkyl, halogen, —CN, —OR¹⁰, —NR¹¹R¹², —S(O)₂R¹⁰, —NR¹⁰S(O)₂R¹¹, —C(O)R¹⁰, —NR¹⁰C(O)R¹¹, or —C(O)NR¹¹R¹², wherein the C₁-C₆ alkyl, —OR¹⁰, —NR¹¹R¹², —S(O)₂R¹⁰, —NR¹⁰S(O)₂R¹¹, —C(O)R¹⁰, —NR¹⁰C(O)R¹¹, and —C(O)NR¹¹R¹² of R⁵ are each independently optionally substituted by halogen, —OR³, or —NR¹³R¹⁴.

20. The compound of any one of claims 1-19, or a salt thereof, wherein q is 0 or 1.

21. The compound of any one of claims 1-20, or a salt thereof, wherein each R⁶ is independently C₁-C₆ alkyl, —OR¹⁰, 3- to 6-membered heterocyclyl, or —NR¹¹R¹², wherein the C₁-C₆ alkyl, —OR¹⁰, 3- to 6-membered heterocyclyl, and —NR¹¹R¹² of R⁶ are each independently optionally substituted by —OR¹³.

22. The compound of claim 1, or a salt thereof, wherein A, L, and B together with R⁵ and R⁶ form a moiety selected from the group consisting of:

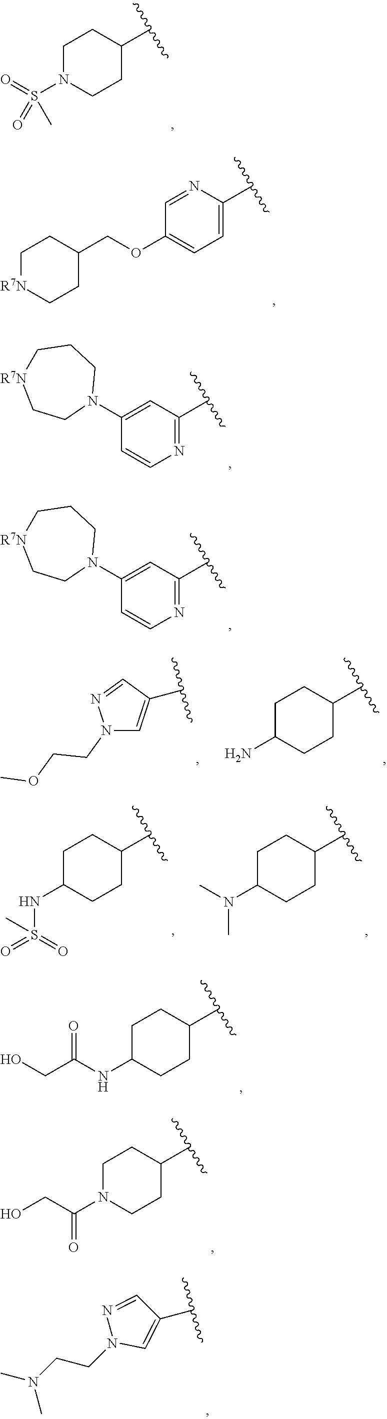

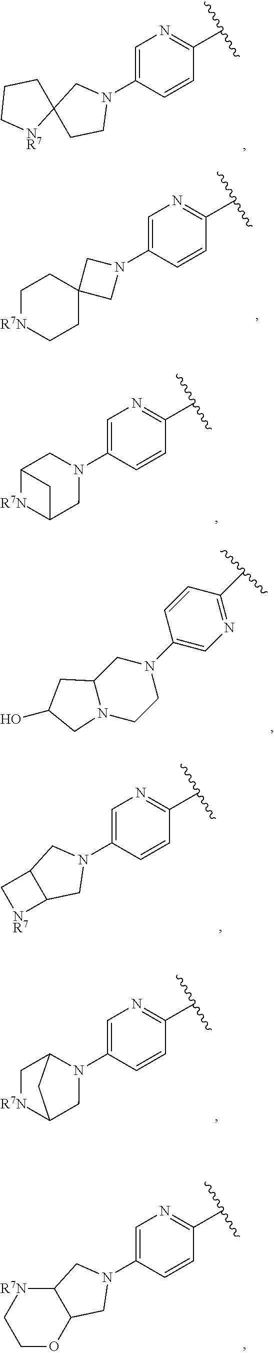

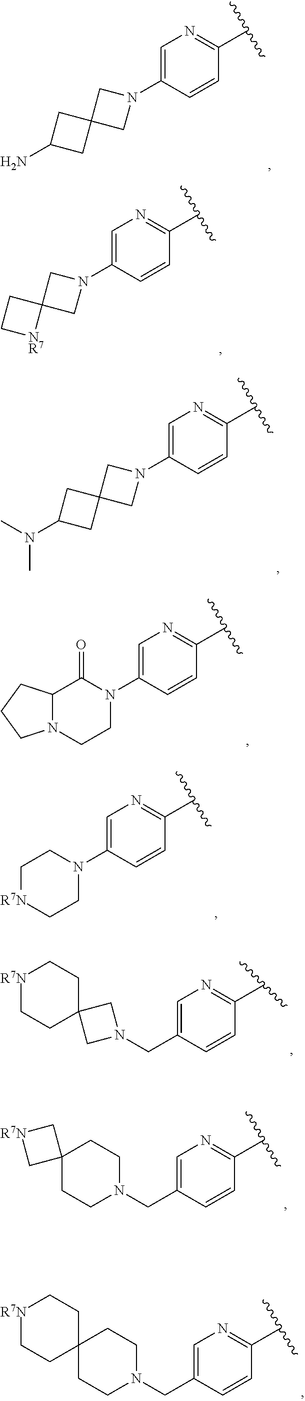

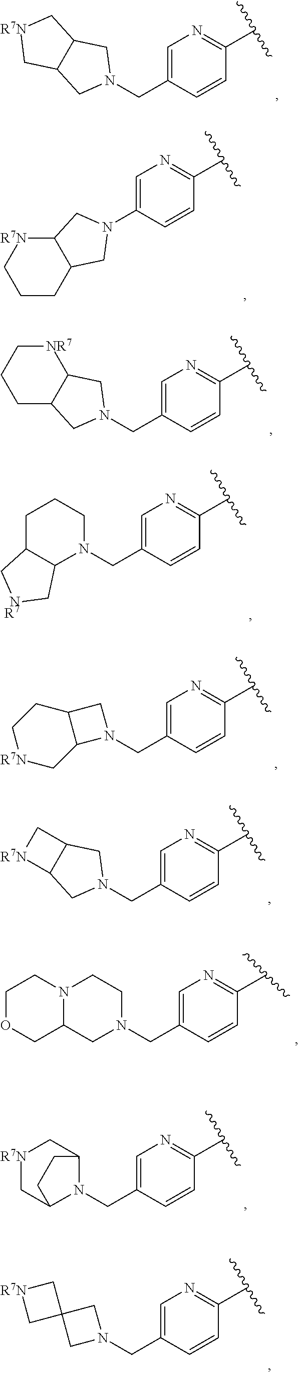

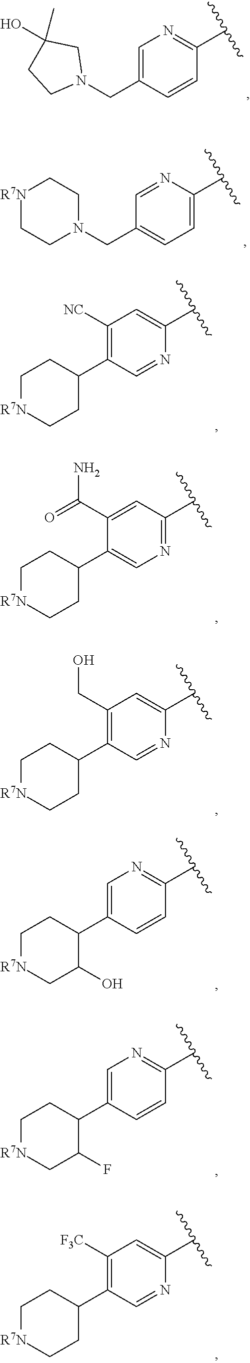

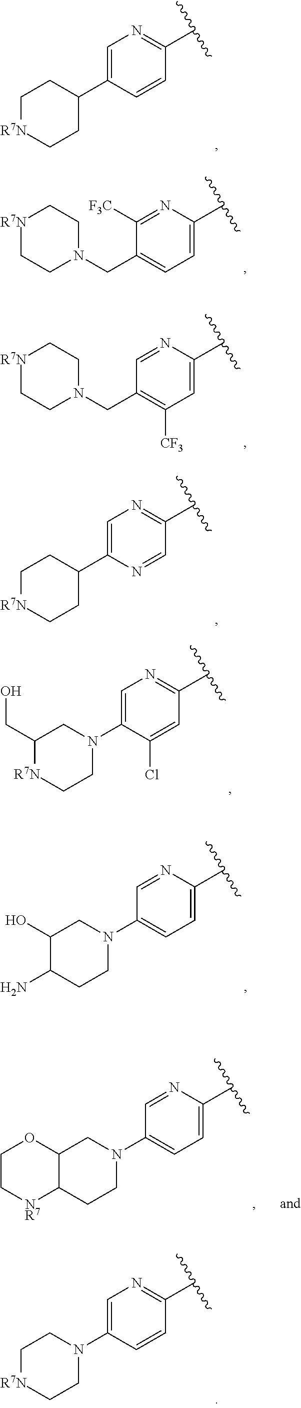

23. The compound of claim 22, or a salt thereof, wherein R⁷ is hydrogen or C₁-C₆ alkyl.

24. The compound of any one of claims 1-23, or a salt thereof, wherein Z is —NH—, —C(O)NH—, or, —NH(CO)—,

25. The compound of any one of claims 1-24, or a salt thereof, wherein Z is —NH—.

26. The compound of any one of claims 1-25, or a salt thereof, wherein X is N.

27. The compound of any one of claims 1-25, or a salt thereof, wherein X is CR^{a}.

28. The compound of any one of claims 1-27, or a salt thereof, wherein R¹ is C₁-C₆ alkyl or C₃-C₆ cycloalkyl, each of which is independently optionally substituted by halogen, —OH, or C₁-C₆ alkyl.

29. The compound of any one of claims 1-28, or a salt thereof, wherein R¹ is

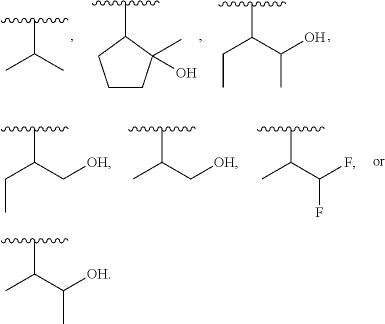

30. The compound of any one of claims 1-29, or a salt thereof, wherein n is 0, 1, or 2.

31. The compound of any one of claims 1-33, or a salt thereof, wherein each R² is independently C₁-C₆ alkyl or halogen.

32. The compound of any one of claims 1-31, or a salt thereof, wherein R⁴ is halogen.

33. The compound of any one of claims 1-32, or a salt thereof, R⁴ is flouro.

34. A pharmaceutical composition comprising the compound of any one of claims 1-33, or a pharmaceutically acceptable salt thereof, and a pharmaceutically acceptable carrier.

35. The pharmaceutical composition of claim 34, wherein the compound is selected from the compounds in Table 1, or a pharmaceutically acceptable salt thereof.

36. A method of treating a cancer in an individual in need thereof comprising administering to the individual a therapeutically effective amount of a compound of any one of claims 1-33, or a pharmaceutically acceptable salt thereof.

37. The method of claim 36, where the cancer is a breast cancer, brain cancer, colorectal cancer, lung cancer, gastric cancer, liver cancer, leukemia, lymphoma, mantle cell lymphoma, melanoma, ovarian cancer, pancreatic cancer, prostate cancer, adult hematopoietic or solid tumor, or pediatric tumor.

38. The method of claim 36 or 37, further comprising administering a radiation therapy to the individual.

39. The method of any one of claims 36-38, further comprising administering to the individual a therapeutically effective amount of a second therapeutic agent.

40. The method of claim 39, wherein the second therapeutic agent is a cancer immunotherapy agent, an endocrine therapy agent, or a chemotherapeutic agent.

41. The method of claim 39 or 40, wherein the second therapeutic agent is a cancer immunotherapy.

42. The method of claim 39 or 40, wherein the second therapeutic agent is an anti-PD-1 antibody.

43. The method of claim 40, wherein the endocrine therapy agent is an antiestrogen therapy, a selective estrogen receptor degrader (SERD), a selective estrogen receptor modulator (SERM) or an aromatase inhibitor.

44. The method of claim 40, wherein the chemotherapeutic agent is a DNA alkylating agent, a platinum-based chemotherapeutic agent, a taxane, a BTK inhibitor, a PI3K inhibitor, another kinase inhibitor, or a DNA damage repair (DDR) pathway inhibitor.

45. The method of any one of claims 36-44, wherein the cancer comprises a mutated or overexpressed CDK gene.

46. The method of any one of claims 36-44, comprising selecting the individual for treatment based on (i) the presence of one or more mutations or amplifications of the CDK4 or CDK6 or other CDK gene in the cancer, (ii) overexpression of CDK4 or CDK6 or other CDK protein in the cancer, (iii) amplification or overexpression of the genes encoding cyclins, (iv) loss of endogenous INK4 inhibitors by gene deletion, mutation, or promoter hypermethylation, (v) other genetic events leading to overactivity of CDK4 or CDK6 or other CDK, or (vi) phosphorylation of retinoblastoma (Rb) protein in the cancer.

47. A method of arresting the G₁-S checkpoint in a cell, comprising administering a compound of any one of claims 1-33, or a salt thereof, to the cell.

48. A method of inhibiting CDK4 or CDK6 in a cell, comprising administering a compound of any one of claims 1-33, or a salt thereof, to the cell.

49. A method of inhibiting CDK4 or CDK6, comprising contacting CDK4 or CDK6 with a compound of any one of claims 1-33, or a salt thereof.

50. The method of claim 48, wherein the inhibitor binds to CDK4 or CDK6 with an IC₅₀ of less than 1 μM according to a kinase assay.

51. Use of a compound of any one of claims 1-33, or a salt thereof, in the manufacture of a medicament for treatment of cancer.

52. A kit comprising a compound of any one of claims 1-33, or a salt thereof.
